(19) United States
(12) Patent Application Publication
(10) Pub. No.: US 2025/0161287 A1
(43) Pub. Date: May 22, 2025

(54) METHODS, COMPOSITIONS, AND COMBINATIONS FOR PREVENTING OR TREATING CANCER RECURRENCE

(71) Applicant: VUJA DE SCIENCES, INC., Hoboken, NJ (US)

(72) Inventors: David Warshawsky, Hoboken, NJ (US); Scott J. Sauer, Waltham, MA (US); Robert Hurst, Oklahoma City, OK (US)

(21) Appl. No.: 18/839,192

(22) PCT Filed: Feb. 17, 2023
(86) PCT No.: PCT/US2023/013285

§ 371 Date: Aug. 16, 2024

Related U.S. Application Data
(60) Provisional application No. 63/311,564, filed on Feb. 18, 2022.
(60) Provisional application No. 63/438,166, filed on Jan. 10, 2023.

(51) Int. Cl. A61K 31/4545; A61K 31/325; A61K 31/4155; A61K 31/453; A61P 35/04

(52) U.S. Cl. CPC A61K 31/4545; A61K 31/325; A61K 31/4155; A61K 31/453; A61P 35/04

(57) ABSTRACT

Methods for screening therapeutic agents for activity against dormant cancer cells, therapeutic agents identified by the methods, and treatment methods related to the same, and methods for screening therapeutic agents for activity against microscopic metastatic tumor cancer cells, therapeutic agents identified by the methods, and treatment methods related to the same.

### CROSS-REFERENCE TO RELATED APPLICATIONS

This application claims the benefit of U.S. Application No. 63/311,564, filed on Feb. 18, 2022, and U.S. Application No. 63/438,166, filed on Jan. 10, 2023, the contents of which are incorporated herein by reference in their entireties.

### BACKGROUND

Most people who die of cancer do not die from the primary tumor. Rather, they succumb to drug-resistant cancer recurrence that has spread from the initial tumor to distant sites such as the liver, lungs, brain, and bones. These recurrences are caused by dormant disseminated tumor cells, micrometastatic cancer cells, and/or suppressed cancer cells. Early in the growth of the primary tumor (typically before oncological treatment), these cells escape and spread to distant sites. These cells may remain dormant at these sites for months, years, or even decades. When they later begin to proliferate, the result is often fatal. In many cancers, including major cancer types such as osteosarcoma (OS), breast cancer, prostate cancer, colon cancer, melanoma, and lung and kidney cancer, recurrence resulting from dormant cancer cells is a major health concern as millions of people who have suffered from these types of cancer are susceptible to recurrence.

These recurrences may also be caused by other microscopic metastatic tumor cells, including persistent disseminated tumor cells, micrometastatic cancer cells, suppressed cancer cells and/or micrometastatic tumors. Microscopic metastatic tumor cells and microscopic tumors result from cancer cells that initially originate from the primary tumor (either before or after oncological treatment, before or after diagnosis of primary cancer), escape and spread to distant, secondary sites, where they can remain microscopic for months, years, or even decades. Similar to dormant cancer cells above, when microscopic metastatic tumor cells begin to proliferate and grow, the result is often fatal. In many cancers, including major cancer types such as osteosarcoma (OS), breast cancer, prostate cancer, colon cancer, melanoma, and lung and kidney cancer, and many pediatric and rare cancers, recurrence resulting from microscopic metastatic tumors, comprised of one cell or clusters of few or many cells that, including persistent disseminated tumor cells, micrometastatic cancer cells, suppressed cancer cells and micrometastatic tumors is a major health concern as millions of people who have suffered from these types of cancer are susceptible to recurrence.

To date, most efforts in cancer therapy focus on killing primary tumors or tumors that have returned and are detectable at distant sites. Drug development efforts often seek to find compounds that target activity of cancer cells or reduce the growth of proliferating tumors. These compounds may target proliferation mechanisms that are general to many cell types or are more unique to cancer cells. These drugs may also induce cell death through mechanisms such as apoptosis. Many cancer treatments involve drugs that cause confining toxicities and side effects. Such treatments can limit the duration of treatment, impose dosing restrictions, and present difficulties with drug combination therapy.

Preventing metastatic recurrence or progression can be achieved by targeting Metastatic Endurance (ME) of microscopic, dormant disseminated tumor cells, persistent disseminated dormant disseminated tumor cells and micrometastatic tumors that originate from the initial tumor and reside undetected at distant sites is a key culprit of cancer recurrence. ME allows these undetected tumor cells to survive and thrive at metastatic sites leading to recurrence.

The initial step in developing an anticancer drug involves screening compounds against cancer cells growing in ordinary tissue culture on plastic. In vitro growth on plastic or in various 2D or 3D systems neglects the true physiological conditions under which tumors grow in the body, specifically tumor growth while in contact with the extracellular matrix. The extracellular matrix (ECM) in living tissues is the supporting material on which cells grow. The ECM interacts with cells to regulate their growth and controls how cells assume mature functions. Cancer cells remodel the ECM to make it more conducive for cancer cell growth, which is essential for tumor formation. Cancer cells that have escaped from the primary tumor exist in a 3D microenvironment within the secondary organs that they inhabit. The normal ECM and other factors in this 3D microenvironment have a major role in maintaining those cells in a dormant state for extended periods of time, particularly during cancer remission, or cancer-free periods.

There remains a need for treatments that target and disable dormant disseminated tumor cells, micrometastatic cancer cells, and suppressed cancer cells. Specifically, a need exists for in vitro drug discovery systems that mimic real-life 3D conditions of dormant cancer cells. Treatment based on such systems can be started and used to effectively prevent cancer recurrence at distant sites. The target oncology product profile for disabling these cancer cells before they can cause recurrence is a low-dose, long-term, and safe drug. Without wishing to be bound by theory, the drug may be administered in various doses and in various dosing regimens, and kills these cancer cells, attenuates the growth of such dormant disseminated tumor cells, and/or keeps them from switching to high growth or proliferation.

There also remains a need for treatments that target and disable microscopic metastatic tumor cells, including persistent disseminated tumor cells, micrometastatic cancer cells, suppressed cancer cells, and micrometastatic tumors. Specifically, a need exists for in vitro drug discovery systems that mimic real-life 3D conditions of these tumor cells and micrometastatic tumors. Treatment based on such systems can be started and used to effectively prevent cancer recurrence at distant sites. A target oncology product profile for disabling these cancer cells before they can cause recurrence is a long-term and safe drug that can be administered in various doses and in various dosing regimens. Without wishing to be bound by theory, the drug can kill such cells, attenuate the growth of such dormant disseminated tumor cells, and/or keep them from switching to high growth or proliferation.

### SUMMARY

The present disclosure is based on methods that utilize in vitro systems to mimic real life 3D conditions of dormant cancer cells. Basement Membrane Extract (BME) is a soluble form of basement membrane purified from Engelbreth-Holm-Swarm (EHS). Porcine small intestine submucosa gel (SISgel) is a soluble form of gel derived from healthy stroma of porcine and provides a suppressive normal ECM proxy. BME and SISgel, among other compositions, can be used to culture various types of cancer cells in conditions that mimic the conditions that occur in the body and specifically at the ECM. Ex vivo systems can also be used to culture various types of cancer cells in conditions that mimic the conditions that occur in the body and specifically at the ECM. One such system is the ex vivo Pulmonary Metastasis Assay (PuMA). Using these compositions in in vitro and ex vivo systems, it is possible to generate dormancy and growth profiles, phenotypes, and biological behaviors of various metastatic cancer cell types in the body and to test the activity of therapeutic agents on such cells.

Thus, in accordance with the purpose(s) of the invention, as embodied and broadly described herein, the disclosure, in one aspect, relates to screening methods, therapeutic agents identified by those screening methods, and methods for using the identified therapeutic agents to target, disable, or otherwise inhibit growth or proliferation of dormant cancer cells, micrometastatic cancer cells, and suppressed cancer cells, thereby preventing cancer recurrence.

In one aspect, described is a method of identifying a candidate therapeutic that has selective activity against metastatic progression of a dormant cancer cell comprising: (a) obtaining or having obtained highly metastatic cells; (b) culturing the highly metastatic cells in a 3D matrix under conditions to impose a metastatic endurance phenotype in the highly metastatic cells, wherein the metastatic endurance phenotype is determined by comparing the change in the total number of cells in the culture daily for at least six days and comparing the change in the total number of cells from a first period of days and a second period of days, wherein an increase in proliferation between the second period of days as compared to the first period of days indicates a metastatic endurance phenotype; (c) culturing the highly metastatic cells in a 3D matrix under conditions to impose a metastatic endurance phenotype in the highly metastatic cells and contacting the highly metastatic cells with a candidate therapeutic; (d) culturing the highly metastatic cells in a 3D matrix under conditions to impose a metastatic endurance phenotype in the highly metastatic cells and contacting the highly metastatic cells with a control; (e) determining the change in the change in the total number of cells from step c) and step d) over the time course; (f) comparing the change in the change in the total number of cells from step c) to the change in the total number of cells of a step d) after at least six days of culture; (g) identifying a candidate therapeutic capable of preventing or inhibiting proliferation of a cell with a metastatic endurance phenotype, when the change in the total number of cells from step c) is the same or less than the change in the total number of cells from step d) after six days of culture; (h) culturing the highly metastatic cells from step b) in a 2D culture under conditions that promote proliferation, contacting the cultured proliferating highly metastatic cells with a candidate therapeutic such as small molecule compound, a biologic or a cell such as an NK cell or other immune cell, or any other biologically active substance; (i) determining the change in the change in the total number of cells in the 2D culture of step h); (j) comparing the change in the change in the total number of cells of step i) with the change in the change in the total number of cells from step c); wherein when the candidate therapeutic reduces the change in the total number of cells in step c) to a greater extent compared to the change in the total number of cells of step i), the candidate therapeutic has selective activity against a metastatic progression of a dormant cancer cell.

In one aspect, described is a method of identifying a candidate therapeutic that has activity against a highly metastatic cell, the method comprising: (a) obtaining or having obtained highly metastatic cells; (b) culturing a first population of the highly metastatic cells in a 3D matrix under conditions sufficient to impose a metastatic endurance phenotype in the highly metastatic cells in the presence of a negative control, wherein the metastatic endurance phenotype is determined by measuring the total number of cells in the culture on at least three time points and determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under conditions that are substantially similar to those used to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; and (f) comparing the total number of cells from step b) and step c) on at least the third time point of step b), wherein when the total number of cells in the third time point of step c) is lower than the total number of cells in the third time point of step b), the candidate therapeutic has activity against a highly metastatic cell.

In one aspect, described is a method of identifying a candidate therapeutic that has selective or specific activity against metastatic progression of a dormant cancer cell comprising: (a) obtaining or having obtained highly metastatic cells; (b) culturing a first population of the highly metastatic cells in a 3D matrix under conditions sufficient to impose a metastatic endurance phenotype in the highly metastatic cells in the presence of a negative control, wherein the metastatic endurance phenotype is determined by measuring the total number of cells in the culture on at least three time points and determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; (f) comparing the total number of cells from the third time point in step c) to the total number of cells from the third time point in step b), calculating the difference in the total number of cells as a ratio by [(third time point of step c−second time point of step b)/(third time point of step b−second time point of step b)]×100% or 100%−[(third time point of step c−second time point of step b)/(third time point of step b−second time point of step b)]×100; (g) identifying a candidate therapeutic capable of preventing or inhibiting proliferation, or capable of preventing, delaying, or reducing a switch or change to proliferation of a cell with a metastatic endurance phenotype, when the ratio of change in the total number of cells from step c) is less than the ratio of change in the total number of cells from step b) between the second and third time points; (h) culturing a fourth population of the highly metastatic cells from step a) in a 2D culture under conditions that promote proliferation and contacting the cultured proliferating highly metastatic cells with the candidate therapeutic from step c); (i) culturing a fifth population of the highly metastatic cells from step a) in a 2D culture under conditions that promote proliferation and contacting the cultured proliferating highly metastatic cells with the negative control from step b); (j) determining the total number of cells in the 2D cultures of step h) compared to step i) at the same time point and calculating a change in the number of cells as a ratio; and (k) comparing the ratio of step j) with the ratio of step f), wherein when the ratio is higher from step f) compared to step j) the candidate therapeutic has selective or specific activity against metastatic progression of a dormant cancer cell.

In one aspect, described is a method of identifying a candidate therapeutic that has selective or specific activity against metastatic progression of a dormant cancer cell comprising: (a) obtaining or having obtained highly metastatic cells; (b) culturing a first population of the highly metastatic cells in a 3D matrix under conditions sufficient to impose a metastatic endurance phenotype in the highly metastatic cells in the presence of a negative control, wherein the metastatic endurance phenotype is determined by measuring the total number of cells in the culture on at least three time points and determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step c) is 75% or less the total number of cells from step b) indicates a metastatic endurance phenotype; (f) comparing the total number of cells from step b) and step c) on at least the third time point of step b) and calculating the % inhibition between the second and third time points by the equation: 100%−[(total # of cells at the third time point of step c)−(total # of cells at the second time point of step b)]/[(total # of cells the third time point of step b)−(total # of cells at the second time point of step b)]×100%; (g) culturing a fourth population of the highly metastatic cells from step a) in a 2D culture under conditions that promote proliferation and contacting the cultured proliferating highly metastatic cells with the candidate therapeutic from step c); (h) culturing a fifth population of the highly metastatic cells from step a) in a 2D culture under conditions that promote proliferation and contacting the cultured proliferating highly metastatic cells with the negative control from step b); (i) determining the total number of cells in the 2D cultures of step g) and h) at approximately the same time point and calculating % inhibition in the 2D cultures by the equation: 100%−[(the total number of cells from step g)/(the total number of cells from step h)×100%]; and (j) comparing the ratio of step i) with the ratio of step f), wherein a lower ratio of step f) compared to step i) identifies selective or specific activity in 3D vs 2D, indicating that the candidate therapeutic has selective or specific activity against metastatic progression of a dormant cancer cell.

In one aspect, described is a method of identifying a candidate therapeutic, the method comprising: (a) obtaining or having obtained highly metastatic cells; (b) culturing a first population of the highly metastatic cells in a 3D matrix under conditions to impose a metastatic endurance phenotype in the highly metastatic cells in the presence of a negative control, wherein the metastatic endurance phenotype is determined by measuring the total number of cells in the culture on at least three time points, determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; and (f) comparing the total number of cells from step b) and step c) on at least the third time point of step b), wherein when the total number of cells in the third time point of step c) is lower than the total number of cells in the third time point of step b), the candidate therapeutic has activity against a highly metastatic cell.

In one aspect, described is a method of identifying a candidate therapeutic that has selective or specific activity against metastatic progression of a microscopic metastatic tumor comprising: (a) obtaining or having obtained highly metastatic cells; (b) culturing a first population of the highly metastatic cells in a 3D matrix under conditions to impose a metastatic endurance phenotype in the highly metastatic cells in the presence of a negative control, wherein the metastatic endurance phenotype is determined by measuring the total number of cells in the culture on at least three time points, determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; (f) comparing the total number of cells from the third time point in step c) to the total number of cells from the third time point in step b), calculating the difference in the total number of cells as a ratio by [(third time point of step c−second time point of step b)/(third time point of step b−second time point of step b)]×100% or 100%−[(third time point of step c−second time point of step b)/(third time point of step b−second time point of step b)]×100; (g) identifying a candidate therapeutic capable of preventing or inhibiting proliferation, or capable of preventing, delaying or reducing a switch, or change to proliferation of a cell with a metastatic endurance phenotype, when the ratio of change in the total number of cells from step c) is less than the ratio of change in the total number of cells from step b) between the second and third time points; (h) culturing a fourth population of the highly metastatic cells from step a) in a 2D culture under conditions that promote proliferation and contacting the cultured proliferating highly metastatic cells with the candidate therapeutic from step c); (i) culturing a fifth population of the highly metastatic cells from step a) in a 2D culture under conditions that promote proliferation and contacting the cultured proliferating highly metastatic cells with the negative control from step b); (j) determining the total number of cells in the 2D cultures of step h) compared to step i) at the same time point and calculating a change in the number of cells as a ratio; and (k) comparing the ratio of step j) with the ratio of step f), wherein when the ratio is higher from step f) compared to step j) then the candidate therapeutic has selective or specific activity against metastatic progression of a microscopic metastatic tumor.

In one aspect, described is a method of identifying a candidate therapeutic that has selective or specific activity against metastatic progression of a microscopic metastatic tumor comprising: (a) obtaining or having obtained highly metastatic cells; (b) culturing a first population of the highly metastatic cells in a 3D matrix under conditions to impose a metastatic endurance phenotype in the highly metastatic cells in the presence of a negative control, wherein the metastatic endurance phenotype is determined by measuring the total number of cells in the culture on at least three time points, determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step c) is 75% or less the total number of cells from step b) indicates a metastatic endurance phenotype; (f) comparing the total number of cells from step b) and step c) on at least the third time point of step b) and calculating the % inhibition between the second and third time points by the equation: 100%−[(total # of cells at the third time point of step c)−(total # of cells at the second time point of step b)]/[(total # of cells the third time point of step b)−(total # of cells at the second time point of step b)]×100%; (g) culturing a fourth population of the highly metastatic cells from step a) in a 2D culture under conditions that promote proliferation and contacting the cultured proliferating highly metastatic cells with the candidate therapeutic from step c); (h) culturing a fifth population of the highly metastatic cells from step a) in a 2D culture under conditions that promote proliferation and contacting the cultured proliferating highly metastatic cells with the negative control from step b); (i) determining the total number of cells in the 2D cultures of step g) and h) at the same time point and calculating % inhibition in the 2D cultures by the equation: 100%−[(the total number of cells from step g)/(the total number of cells from step h)×100%]; and (j) comparing the ratio of step i) with the ratio of step f), wherein a lower ratio of step f), compared to step i) identifies selective or specific activity in 3D vs 2D, indicating that the candidate therapeutic has selective or specific activity against metastatic progression of a microscopic metastatic tumor.

In one aspect, the described methods are useful for identifying a candidate therapeutic that has selective activity against metastatic progression of a microscopic tumor.

In one aspect, this method is useful for identifying combinations of candidate therapeutics that have selective activity against metastatic progression of a dormant cancer cell and/or a microscopic tumor, wherein two or more drugs are tested in combination. The effect of a combination is compared to the effect of each drug used in the combination alone, and the effect of the combination is determined using the Highest Single Agent approach. The Highest Single Agent approach compares the activity of the strongest drug in the combination alone relative to the effect of the combination. See, e.g., Foucquier et al. PRP, 2015. Greater inhibition with the combination relative to the strongest single agent is said to exhibit a combinatorial effect.

In a further aspect, described is a method of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of an active pharmaceutical ingredient (API) selected from:
mTOR inhibitors;
heat shock protein 90 (HSP90) inhibitors;
heat shock protein 70 (HSP70) inhibitors;
unfolded protein response (UPR) inhibitors;
modulators of nicotinamide adenine dinucleotide (NAD⁺) metabolism;
a glutamyl-prolyl tRNA synthetase inhibitors;
glutaminase inhibitors;
poly(ADP)-ribose polymerase (PARP) inhibitors;
DNA-damaging agents;
agents capable of generating reactive oxygen species (ROS);
survivin inhibitors;
MEK 1 and/or MEK 2 inhibitors;
BCL-XL and/or BCL-2 inhibitors;
proteosome inhibitors;
muscarinic acetylcholine (mACh) antagonists;
B-Raf inhibitors;
Bromodomain (BET) protein inhibitors;
AXL kinase inhibitors;
NEDD8 inhibitors;
aurora kinase inhibitors;
histone deacetylase (HDAC) inhibitors (e.g., Class I selective histone deacetylase (HDAC) inhibitors);
PI3K inhibitors;
Akt inhibitors;
LSD inhibitors;
DNA-PK inhibitors;
IGF1R inhibitors; and
PLK inhibitors.

In a further aspect, described is a method of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of a class 1 selective HDAC inhibitor.

In a further aspect, described is a method of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of a drug selected from an active pharmaceutical ingredient (API), a large molecule, an antibody, a nucleic acid based drug, an aptamer, a peptide or a protein that inhibits the activity of epigenome modulators or regulators.

In a further aspect, described is a method of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of two or more active pharmaceutical ingredients (APIs) selected from:
a mTOR inhibitor;
a heat shock protein 90 (HSP90) inhibitor;
a heat shock protein 70 (HSP70) inhibitor;
an unfolded protein response (UPR) inhibitor;
a modulator of nicotinamide adenine dinucleotide (NAD+) metabolism;
a glutamyl-prolyl tRNA synthetase inhibitor;
a glutaminase inhibitor;
a apoly(ADP)-ribose polymerase (PARP) inhibitor;
DNA-damaging agent;
an agent capable of generating reactive oxygen species (ROS);
a survivin inhibitors;
a MEK 1 and/or MEK 2 inhibitor;
a Bcl-xL and/or Bcl-2 inhibitor;
a proteosome inhibitor;
a muscarinic acetylcholine (mACh) antagonist;
a B-Raf inhibitor;
a BET protein inhibitor;
an AXL kinase inhibitor;
a NEDD8 inhibitor;
an aurora kinase inhibitor;
a histone deacetylase (HDAC) inhibitor;
a PI3K inhibitor;
an Akt inhibitor;
a LSD inhibitor;
a DNA-PK inhibitor;
an IGF1R inhibitor; and
a PLK inhibitor.

In a further aspect, described is a method of preventing, treating, or ameliorating in a patient in need thereof, a microscopic metastatic tumor comprised of one cell or clusters of a few or many cells that, including persistent disseminated tumor cells, micrometastatic cancer cells, suppressed cancer cells and/or micrometastatic tumors by preventing or inhibiting proliferation of a dormant, suppressed, not growing, slow growing cancer cell, or inhibiting the transitioning of such cells to a rapidly proliferating cancer or rapidly growing tumor, or killing such cells, the method comprising contacting a cell or cells of the microscopic metastatic tumor with an effective amount of an active pharmaceutical ingredient (API) selected from:
mTOR inhibitors;
heat shock protein 90 (HSP90) inhibitors;
heat shock protein 70 (HSP70) inhibitors;
unfolded protein response (UPR) inhibitors;
modulators of nicotinamide adenine dinucleotide (NAD⁺) metabolism;
a glutamyl-prolyl tRNA synthetase inhibitors;
glutaminase inhibitors;
poly(ADP)-ribose polymerase (PARP) inhibitors;
DNA-damaging agents;
agents capable of generating reactive oxygen species (ROS);
survivin inhibitors;
MEK 1 and/or MEK 2 inhibitors;
BCL-XL and/or BCL-2 inhibitors;
proteosome inhibitors;
muscarinic acetylcholine (mACh) antagonists;
B-Raf inhibitors;
BET protein inhibitors;
AXL kinase inhibitors;
NEDD8 inhibitors;
aurora kinase inhibitors;
histone deacetylase (HDAC) inhibitors (e.g., Class I selective histone deacetylase (HDAC) inhibitors);
PI3K inhibitors;
Akt inhibitors;
LSD inhibitors;
DNA-PK inhibitors;
IGF1R inhibitors; and
PLK inhibitors.

In a further aspect, described is a method of preventing, treating, or ameliorating in a patient in need thereof, a microscopic metastatic tumor comprised of one cell or clusters of few or many cells that, including persistent, dormant disseminated tumor cells, micrometastatic cancer cells, suppressed cancer cells, and/or micrometastatic tumors by preventing or inhibiting proliferation of a dormant, suppressed, not growing, slow growing cancer cell, or inhibiting the transitioning of such cells to a rapidly proliferating cancer or rapidly growing tumor, or killing such cells, the method comprising contacting a cell or cells of the microscopic metastatic tumors with an effective amount of combination of two or more active pharmaceutical ingredients (APIs) selected from the list above.

In a further aspect, described are methods of preventing, treating, or ameliorating in a patient in need, such patient being a patient diagnosed with a cancer and having current minimal residual disease, or the patient being diagnosed with cancer and with undetected minimal residual disease and such patient being at high risk for cancer recurrence or cancer progression as determined by one of a variety of different factors including, but not limited to, the type of cancer, the stage of detection, family history, genetic factors and biomarkers that a clinician may utilize for assessing such risk, such patient with or without prior treatment.

In a still further aspect, described is a method of targeting minimal residual disease or preventing recurrence of cancer in a subject in need thereof, the method comprising administering to the subject an effective amount of an active pharmaceutical ingredient (API) selected from:
mTOR inhibitors;
heat shock protein 90 (HSP90) inhibitors;
heat shock protein 70 (HSP70) inhibitors;
unfolded protein response (UPR) inhibitors;
modulators of nicotinamide adenine dinucleotide (NAD⁺) metabolism;
a glutamyl-prolyl tRNA synthetase inhibitors;
glutaminase inhibitors;
poly(ADP)-ribose polymerase (PARP) inhibitors;
DNA-damaging agents;
agents capable of generating reactive oxygen species (ROS);
survivin inhibitors;
MEK 1 and/or MEK 2 inhibitors;
BCL-XL and/or BCL-2 inhibitors;
proteosome inhibitors;
muscarinic acetylcholine (mACh) antagonists;
B-Raf inhibitors;
BET protein inhibitors;
AXL kinase inhibitors;
NEDD8 inhibitors;
aurora kinase inhibitors;
histone deacetylase (HDAC) inhibitors (e.g., Class I selective histone deacetylase (HDAC) inhibitors);
PI3K inhibitors;
Akt inhibitors;
LSD inhibitors;
DNA-PK inhibitors;
IGF1R inhibitors; and
PLK inhibitors.

In a still further aspect, described is a method of targeting minimal residual disease or preventing recurrence of cancer in a subject in need thereof, the method comprising administering to the subject an effective amount of two or more active pharmaceutical ingredients (APIs) selected from:
mTOR inhibitors;
heat shock protein 90 (HSP90) inhibitors;
heat shock protein 70 (HSP70) inhibitors;
unfolded protein response (UPR) inhibitors;
modulators of nicotinamide adenine dinucleotide (NAD^{m}) metabolism;
a glutamyl-prolyl tRNA synthetase inhibitors;
glutaminase inhibitors;
poly(ADP)-ribose polymerase (PARP) inhibitors;
DNA-damaging agents;
agents capable of generating reactive oxygen species (ROS);
survivin inhibitors;
MEK 1 and/or MEK 2 inhibitors;
BCL-XL and/or BCL-2 inhibitors;
proteosome inhibitors;
muscarinic acetylcholine (mACh) antagonists;
B-Raf inhibitors;
BET protein inhibitors;
AXL kinase inhibitors;
NEDD8 inhibitors;
aurora kinase inhibitors;
histone deacetylase (HDAC) inhibitors (e.g., Class I selective histone deacetylase (HDAC) inhibitors);
PI3K inhibitors;
Akt inhibitors;
LSD inhibitors;
DNA-PK inhibitors;
IGF1R inhibitors; and
PLK inhibitors.

In a further aspect, described is a method of targeting minimal residual disease or preventing recurrence of cancer in a subject in need thereof, the method comprising administering to the subject an effective amount of an active pharmaceutical ingredient (API), an antibody, or an immunotherapeutic that inhibits the activity of epigenome modulators or regulators.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 is a bar graph showing representative data for in vitro 3D BME assay and 2D assays with MG63.3 OS cell line with rapamycin. Highly metastatic OS MG63.3 (human) treated with rapamycin (100 pM-10 μM) lead to appreciable inhibition of 3D growth at doses as low as 500 pM (bars−replicates=4; P<0.005 for all doses ≥500 pM, all comparisons made between treatment and vehicle control). The effect of rapamycin on 2D growth (15 nM-100 μM) over 72 h was measured by MTT (dots−replicates=3), with rapamycin only exhibiting cytotoxicity at doses ≥33 μM. Rapamycin treatment exhibited much stronger activity on 3D BME at significantly lower doses.

FIG. 2 is a bar graph showing representative data for in vitro 3D BME assay and 2D assays with MG63.3 OS cell line with ridaforolimus. Highly metastatic OS MG63.3 (human) treated with ridaforolimus (1-64 nM) lead to appreciable inhibition of 3D growth at doses as low as 1 nM (bars−replicates=4; P<0.005 for all doses ≥1 nM, all comparisons made between treatment and vehicle control). The effect of ridaforolimus on 2D growth (15 nM-100 μM) over 72 h was measured by MTT (dots−replicates=3), with ridaforolimus only exhibiting cytotoxicity at doses >33 μM. Ridaforolimus treatment exhibited much stronger activity on 3D BME at significantly lower doses.

FIG. 3 is a bar graph showing representative data for in vitro 3D BME assay and 2D assays with MG63.3 OS cell line with temsirolimus. Highly metastatic OS MG63.3 (human) treated with temsirolimus (1-64 nM) lead to appreciable inhibition of 3D growth at doses as low as 1 nM (bars−replicates=4; P<0.005 for all doses ≥1 nM, all comparisons made between treatment and vehicle control). The effect of temsirolimus on 2D growth (15 nM-100 μM) over 72 h was measured by MTT (dots−replicates=3), with temsirolimus only exhibiting cytotoxicity at doses >33 μM. Temsirolimus treatment exhibited much stronger activity on 3D BME at significantly lower doses.

FIG. 4 is a bar graph showing representative data for in vitro 3D BME assay and 2D assays with MG63.3 OS cell line with vistusertib. Highly metastatic OS MG63.3 (human) treated with vistusertib (1 pM-100 nM) lead to appreciable inhibition of 3D growth at 100 nM (bars−replicates=4; P<0.005, all comparisons made between treatment and vehicle control). The effect of vistusertib on 2D growth (25 pM-10 μM) over 72 h was measured by MTT (dots−replicates=3), with vistusertib only exhibiting cytotoxicity at doses>640 pM. Vistusertib treatment exhibited slightly stronger activity on 3D BME at 100 nM.

FIG. 5 is a bar graph showing representative data for in vitro 3D BME assay and 2D assays with MG63.3 OS cell line with sapanisertib. Highly metastatic OS MG63.3 (human) treated with sapanisertib (1-64 nM) lead to appreciable inhibition of 3D growth at doses as low as 1 nM (bars−replicates=4; P<0.05 for all doses≥1 nM, all comparisons made between treatment and vehicle control). The effect of sapanisertib on 2D growth (25 pM-10 μM) over 72 h was measured by MTT (dots−replicates=3), with sapanisertib only exhibiting cytotoxicity at doses>8 nM. Sapanisertib treatment exhibited much stronger activity on 3D BME at slightly lower doses.

FIG. 6 is a bar graph showing representative data for in vitro 3D BME assay and 2D assays with MG63.3 OS cell line with everolimus. Highly metastatic OS MG63.3 (human) treated with everolimus (10 nM-10 μM) lead to appreciable inhibition of 3D growth at doses as low as 10 nM (bars−replicates=4; P<0.005 for all doses ≥10 nM, all comparisons made between treatment and vehicle control). The effect of everolimus on 2D growth (25 pM-100 μM) over 72 h was measured by MTT (dots−replicates=3), with everolimus exhibiting cytotoxicity across all doses tested. However, everolimus treatment exhibited much stronger activity on 3D BME at similar doses.

FIG. 7 is a bar graph showing representative data for in vitro 3D BME assay and 2D assays with MG63.3 OS cell line with AZD8055. Highly metastatic OS MG63.3 (human) treated with AZD8055 (1 nM-10 μM) lead to appreciable inhibition of 3D growth at doses as low as 100 nM (bars−replicates=4; P<0.005 for all doses ≥100 nM, all comparisons made between treatment and vehicle control). The effect of AZD8055 on 2D growth (3.125 nM-400 nM) over 72 h was measured by MTT (dots−replicates=3), with AZD8055 only exhibiting cytotoxicity at doses >100 nM. However, AZD8055 treatment exhibited much stronger activity on 3D BME at slightly lower doses.

FIG. 8 is a bar graph showing representative data for in vitro 3D BME assay and 2D assays with MG63.3 OS cell line with telaglenastat. Highly metastatic OS MG63.3 (human) treated with telaglenastat (1.56-100 nM) lead to appreciable inhibition of 3D growth at doses as low as 1 nM (bars−replicates=4; P<0.005 for all doses ≥1.56 nM, all comparisons made between treatment and vehicle control). The effect of telaglenastat on 2D growth (1 pM-10 μM) over 72 h was measured by MTT (dots−replicates=3), with telaglenastat only exhibiting cytotoxicity at doses >100 nM. Telaglenastat treatment exhibited much stronger activity on 3D BME at significantly lower doses.

FIG. 9 is a bar graph showing representative data for in vitro 3D BME assay and 2D assays with MG63.3 OS cell line with pevonedistat. Highly metastatic OS MG63.3 (human) treated with pevonedistat (10-50 nM) lead to appreciable inhibition of 3D growth at doses as low as 1 nM (bars−replicates=4; P<0.005 for all doses ≥10 nM, all comparisons made between treatment and vehicle control). The effect of pevonedistat on 2D growth (3.125-400 nM) over 72 h was measured by MTT (dots−replicates=3), with pevonedistat only exhibiting cytotoxicity at doses >10 nM. Pevonedistat treatment exhibited much stronger activity on 3D BME at significantly lower doses.

FIG. 10 is a bar graph showing representative data for in vitro 3D BME assay and 2D assays with MG63.3 OS cell line with BIIB021. Highly metastatic OS MG63.3 (human) treated with BIIB021 (8-128 nM) lead to appreciable inhibition of 3D growth at doses as low as 8 nM (bars−replicates=4; P<0.005 for all doses ≥8 nM, all comparisons made between treatment and vehicle control). The effect of BIIB021 on 2D growth (3.125-400 nM) over 72 h was measured by MTT (dots−replicates=3), with BIIB021 only exhibiting cytotoxicity at doses >10 nM. BIIB021 treatment exhibited much stronger activity on 3D BME at significantly lower doses.

FIG. 11 is a bar graph showing representative data for in vitro 3D BME assay and 2D assays with MG63.3 OS cell line with alvespimycin. Highly metastatic OS MG63.3 (human) treated with alvespimycin (12.5-200 nM) lead to appreciable inhibition of 3D growth at doses as low as 12.5 nM (bars−replicates=4; P<0.005 for all doses ≥12.5 nM, all comparisons made between treatment and vehicle control). The effect of alvespimycin on 2D growth (3.125-400 nM) over 72 h was measured by MTT (dots−replicates=3), with alvespimycin only exhibiting cytotoxicity at doses >25 nM. Alvespimycin treatment exhibited much stronger activity on 3D BME at significantly lower doses.

FIG. 12 is a bar graph showing representative data for in vitro 3D BME assay and 2D assays with MG63.3 OS cell line with tanespimycin. Highly metastatic OS MG63.3 (human) treated with tanespimycin (25-400 nM) lead to appreciable inhibition of 3D growth at doses as low as 25 nM (bars−replicates=4; P<0.005 for all doses ≥25 nM, all comparisons made between treatment and vehicle control). The effect of tanespimycin on 2D growth (3.125-400 nM) over 72 h was measured by MTT (dots−replicates=3), with tanespimycin only exhibiting cytotoxicity at doses >25 nM. Tanespimycin treatment exhibited much stronger activity on 3D BME at significantly lower doses.

FIG. 13 is a bar graph showing representative data for in vitro 3D BME assay and 2D assays with MG63.3 OS cell line with retaspimycin. Highly metastatic OS MG63.3 (human) treated with retaspimycin (200-400 nM) lead to appreciable inhibition of 3D growth at doses as low as 200 nM (bars−replicates=4; P<0.005 for all doses ≥200 nM, all comparisons made between treatment and vehicle control). The effect of retaspimycin on 2D growth (1 pM-10 μM) over 72 h was measured by MTT (dots−replicates=3), with retaspimycin only exhibiting cytotoxicity at doses >400 nM. Retaspimycin treatment exhibited much stronger activity on 3D BME at significantly lower doses.

FIG. 14 is a bar graph showing representative data for in vitro 3D BME assay and 2D assays with MG63.3 OS cell line with talazoparib. Highly metastatic OS MG63.3 (human) treated with talazoparib (12.5-400 nM) lead to appreciable inhibition of 3D growth at doses as low as 12.5 nM (bars−replicates=4; P<0.005 for all doses ≥12.5 nM, all comparisons made between treatment and vehicle control). The effect of talazoparib on 2D growth (1 pM-10 μM) over 72 h was measured by MTT (dots−replicates=3), with talazoparib only exhibiting cytotoxicity at doses >100 nM. Talazoparib treatment exhibited much stronger activity on 3D BME at significantly lower doses.

FIG. 15 is a bar graph showing representative data for in vitro 3D BME assay and 2D assays with MG63.3 OS cell line with mitomycin C. Highly metastatic OS MG63.3 (human) treated with mitomycin C (12.5-400 nM) lead to appreciable inhibition of 3D growth at doses as low as 12.5 nM (bars−replicates=4; P<0.005 for all doses ≥12.5 nM, all comparisons made between treatment and vehicle control). The effect of mitomycin C on 2D growth (1 pM-10 μM) over 72 h was measured by MTT (dots−replicates=3), with mitomycin C only exhibiting cytotoxicity at doses >200 nM. Mitomycin C treatment exhibited much stronger activity on 3D BME at significantly lower doses.

FIG. 16 is a bar graph showing representative data for in vitro 3D BME assay and 2D assays with MG63.3 OS cell line with alisertib. Highly metastatic OS MG63.3 (human) treated with alisertib (12.5-200 nM) lead to appreciable inhibition of 3D growth at doses as low as 12.5 nM (bars−replicates=4; P<0.005 for all doses ≥12.5 nM, all comparisons made between treatment and vehicle control). The effect of alisertib on 2D growth (1.5625-200 nM) over 72 h was measured by MTT (dots−replicates=3), with alisertib only exhibiting cytotoxicity at doses >25 nM. Alisertib treatment exhibited much stronger activity on 3D BME at significantly lower doses.

FIG. 17 is a bar graph showing representative data for in vitro 3D BME assay and 2D assays with MG63.3 OS cell line with danusertib. Highly metastatic OS MG63.3 (human) treated with danusertib (10-100 nM) lead to appreciable inhibition of 3D growth at doses as low as 100 nM (bars−replicates=4; P<0.005 for 100 nM, all comparisons made between treatment and vehicle control). The effect of danusertib on 2D growth (3.125-400 nM) over 72 h was measured by MTT (dots−replicates=3), with danusertib only exhibiting cytotoxicity at doses >100 nM. Danusertib treatment exhibited much stronger activity on 3D BME at significantly lower doses.

FIG. 18 is a bar graph showing representative data for in vitro 3D BME assay and 2D assays with MG63.3 OS cell line with (E)-daporinad. Highly metastatic OS MG63.3 (human) treated with (E)-daporinad (10 pM-1 nM) lead to appreciable inhibition of 3D growth at doses as low as 100 pM (bars−replicates=4; #P<0.005 for ≥100 pM, all comparisons made between treatment and vehicle control). The effect of (E)-daporinad on 2D growth (1 pM-10 μM) over 72 h was measured by MTT (dots−replicates=3), with (E)-daporinad only exhibiting cytotoxicity at doses >100 pM. (E)-Daporinad treatment exhibited much stronger activity on 3D BME at significantly lower doses.

FIG. 19 is a bar graph showing representative data for in vitro SISGel Assay with Highly Metastatic MG63.3 OS Cells line with rapamycin. Highly metastatic OS MG63.3 (human) treated with rapamycin (0.1 pM-100 nM) lead to appreciable inhibition of 3D growth at doses as low as 1 nM (bars−replicates=4; P<0.005 for ≥1 nM, all comparisons made between treatment and vehicle control). The effect of rapamycin on 2D growth (15 nM-100 μM) over 72 h was measured by MTT (dots−replicates=3), with rapamycin only exhibiting cytotoxicity at doses >11 μM. Rapamycin treatment exhibited much stronger activity on 3D SISGel at significantly lower doses.

FIG. 20 is a bar graph showing representative data for in vitro SISGel Assay with Highly Metastatic MG63.3 OS Cells line with temsirolimus. Highly metastatic OS MG63.3 (human) treated with temsirolimus (0.1 pM-10 nM) lead to appreciable inhibition of 3D growth at doses as low as 1 pM (bars−replicates=4; P<0.005 for ≥1 pM, all comparisons made between treatment and vehicle control). The effect of temsirolimus on 2D growth (15 nM-100 μM) over 72 h was measured by MTT (dots−replicates=3), with temsirolimus only exhibiting cytotoxicity at doses >11 μM. Temsirolimus treatment exhibited much stronger activity on 3D SISGel at significantly lower doses.

FIG. 21 is a bar graph showing representative data for in vitro SISGel Assay with Highly Metastatic MG63.3 OS Cells line with ridaforolimus. Highly metastatic OS MG63.3 (human) treated with ridaforolimus (1 pM-10 nM) lead to appreciable inhibition of 3D growth at doses as low as 100 pM (bars−replicates=4; P<0.005 for ≥100 pM, all comparisons made between treatment and vehicle control). The effect of ridaforolimus on 2D growth (15 nM-100 μM) over 72 h was measured by MTT (dots−replicates=3), with ridaforolimus only exhibiting cytotoxicity at doses >33 μM. Ridaforolimus treatment exhibited much stronger activity on 3D SISGel at significantly lower doses.

FIG. 22 is a bar graph showing representative data for In vitro 3D BME assay and 2D assays with K7M2 OS cell line with rapamycin. Highly metastatic OS K7M2 (mouse) treated with rapamycin (1 & 10 nM) lead to appreciable inhibition of 3D growth at both doses (bars−replicates=4; P<0.005 for ≥1 nM, all comparisons made between treatment and vehicle control). The effect of rapamycin on 2D growth (15 nM-100 μM) over 72 h was measured by MTT (dots−replicates=3), with rapamycin treatment leading to significant cytotoxicity at doses ≥10 PM. Rapamycin treatment exhibited much stronger activity on 3D BME at significantly lower doses.

FIG. 23A is a schematic illustrating the metastatic progression of GFP-expressing OS cells conducted by injecting single cells via tail vein injection, followed by lung excision and insufflation 15 mins post-injection. The formation of multicellular colonies in the mouse lung microenvironment can be assessed 14-21 days post-injection. Representative fluorescent images (10×—Right) show diffuse robust signal in untreated and inactive conditions (top) but near complete loss of signal with active treatment (bottom).

FIG. 23B is a bar graph showing representative data for rapamycin (100 nM and 1 pM) usage on highly metastatic MG63.3 human OS cells. Usage of rapamycin led to strong inhibition of ME correlating with loss of GFP signal in the lung tissue (Representative Examples; replicates=3-5; P<0.05 for all doses of all compounds except ridaforolimus; all comparisons made between treatments and vehicle controls).

FIG. 23C is a bar graph showing representative data for ridaforolimus (100 nM and 1 μM) usage on highly metastatic MG63.3 human OS cells. Usage of ridaforolimus led to strong inhibition of ME correlating with loss of GFP signal in the lung tissue (Representative Examples; replicates=3-5; P<0.05 for all doses of all compounds except ridaforolimus; all comparisons made between treatments and vehicle controls).

FIG. 23D is a bar graph showing representative data for temsirolimus (100 nM and 1 μM) usage on highly metastatic MG63.3 human OS cells. Usage of temsirolimus led to strong inhibition of ME correlating with loss of GFP signal in the lung tissue (Representative Examples; replicates=3-5; P<0.05 for all doses of all compounds except ridaforolimus; all comparisons made between treatments and vehicle controls).

FIG. 23E is a bar graph showing representative data for AZD8055 (10 and 100 nM) usage on highly metastatic MG63.3 human OS cells. Usage of AZD8055 led to strong inhibition of ME correlating with loss of GFP signal in the lung tissue (Representative Examples; replicates=3-5; P<0.05 for all doses of all compounds except ridaforolimus; all comparisons made between treatments and vehicle controls).

FIG. 24A is a scatterplot showing representative data in which highly metastatic cell line MG63.3 (human OS) is grown in BME under certain conditions and shows a lag in proliferation for −3 days, followed by a lack of, or less pronounced lag. Rapamycin (1 nM) was used to assess if the cells are exhibiting the correct ME phenotype to determine the specific activity of ME disabling drugs. 1 nM rapamycin treatment is marginally effective, meaning that in this experiment ME was not adequately achieved and this experiment cannot qualify for screening or testing for drugs that disable ME (Replicates=4).

FIG. 24B is a bar graph showing representative data in which highly metastatic cell line MG63.3 (human OS) is grown in BME under certain conditions and shows a lag in proliferation for −3 days, followed by a lack of, or less pronounced lag. Rapamycin (1 nM) was used to assess if the cells are exhibiting the correct ME phenotype to determine the specific activity of ME disabling drugs. 1 nM rapamycin treatment is marginally effective, meaning that in this experiment ME was not adequately achieved and this experiment cannot qualify for screening or testing for drugs that disable ME (Replicates=4).

FIG. 24C is a scatterplot showing representative data in which highly metastatic cell line MG63.3 (human OS) is grown in BME under certain conditions and shows a lag in proliferation for ˜3 days, followed by increased proliferation (outbreak) by Day 6. Rapamycin (1 nM) was used to assess if the cells are exhibiting the correct ME phenotype to determine the specific activity of ME disabling drugs. When the conditions are such that there is an appreciable lab from Days 1-3, followed by a period of faster growth from Days 3-6, 1 nM rapamycin treatment leads to very strong inhibition of Day 6 growth. Taken together, these two representative examples show the importance of growth kinetics on imposing the ME phenotype and the ability of ME-targeting drugs to specifically work under ME-promoting conditions and not during periods of rapid, consistent growth.

FIG. 24D is a bar graph showing representative data in which highly metastatic cell line MG63.3 (human OS) is grown in BME under certain conditions and shows a lag in proliferation for ˜3 days, followed by increased proliferation (outbreak) by Day 6. Rapamycin (1 nM) was used to assess if the cells are exhibiting the correct ME phenotype to determine the specific activity of ME disabling drugs. When the conditions are such that there is an appreciable lab from Days 1-3, followed by a period of faster growth from Days 3-6, 1 nM rapamycin treatment leads to very strong inhibition of Day 6 growth. Taken together, these two representative examples show the importance of growth kinetics on imposing the ME phenotype and the ability of ME-targeting drugs to specifically work under ME-promoting conditions and not during periods of rapid, consistent growth.

FIG. 25A is a scatterplot showing representative data in which highly metastatic OS line K7M2 (murine) was grown in BME under certain conditions and shows a lag in proliferation for −3 days, followed by a lack of, or less pronounced lag. Rapamycin (1 nM) was used to assess if the cells are exhibiting the correct ME phenotype to determine the specific activity of ME disabling drugs. Here, 1 nM rapamycin treatment is not effective, meaning that in this experiment ME was not achieved and this experiment cannot qualify for screening or testing for drugs that disable ME.

FIG. 25B is a bar graph showing representative data in which highly metastatic OS line K7M2 (murine) was grown in BME under certain conditions and shows a lag in proliferation for ˜3 days, followed by a lack of, or less pronounced lag. Rapamycin (1 nM) was used to assess if the cells are exhibiting the correct ME phenotype to determine the specific activity of ME disabling drugs. Here, 1 nM rapamycin treatment is not effective, meaning that in this experiment ME was not achieved and this experiment cannot qualify for screening or testing for drugs that disable ME.

FIG. 25C is a scatterplot showing representative data in which highly metastatic OS line K7M2 (murine) was grown in BME under certain conditions and shows a lag in proliferation for ˜3 days, followed by increased proliferation (outbreak) by Day 6. Rapamycin (1 nM) was used to assess if the cells are exhibiting the correct ME phenotype to determine the specific activity of ME disabling drugs. This experiment shows strong efficacy with 1 nM rapamycin treatment, meaning that in this experiment ME was achieved and this experiment can qualify for screening or testing for drugs that disable ME.

FIG. 25D is a bar graph showing representative data in which highly metastatic OS line K7M2 (murine) was grown in BME under certain conditions and shows a lag in proliferation for ˜3 days, followed by increased proliferation (outbreak) by Day 6. Rapamycin (1 nM) was used to assess if the cells are exhibiting the correct ME phenotype to determine the specific activity of ME disabling drugs. This experiment shows strong efficacy with 1 nM rapamycin treatment, meaning that in this experiment ME was achieved and this experiment can qualify for screening or testing for drugs that disable ME.

FIG. 26A is a scatterplot showing representative data in which high metastatic cell lines MG63.3 (human OS) were grown in SISgel under certain conditions that lead to different proliferation kinetic profiles over a 6-day period. Multiple doses of rapamycin (10-100 nM) can be used to assess if the cells are exhibiting the correct ME phenotype to determine the specific activity of ME disabling drugs. In this example, two different seeding densities led to different kinetic profiles where the lower density led to slow growth followed by outbreak while higher density exhibited sustained fast growth. The effect of both rapamycin concentrations (12.5 & 100 nM) was much greater under conditions promoting ME (lower density−slower initial growth followed by outbreak), meaning that ME was adequately achieved only with the lower seeding density.

FIG. 26B is a bar graph showing representative data in which high metastatic cell lines MG63.3 (human OS) were grown in SISgel under certain conditions that lead to different proliferation kinetic profiles over a 6-day period. Multiple doses of rapamycin (10-100 nM) can be used to assess if the cells are exhibiting the correct ME phenotype to determine the specific activity of ME disabling drugs. In this example, two different seeding densities led to different kinetic profiles where the lower density led to slow growth followed by outbreak while higher density exhibited sustained fast growth. The effect of both rapamycin concentrations (12.5 & 100 nM) was much greater under conditions promoting ME (lower density−slower initial growth followed by outbreak), meaning that ME was adequately achieved only with the lower seeding density.

FIG. 27 is a plot showing representative data in which highly metastatic OS MG63.3 (human) treated with telaglenastat (1.56-100 nM) lead to appreciable inhibition of 3D growth at doses as low as 1 nM (bars−replicates=4; P<0.005 for all doses ≥1.56 nM, all comparisons made between treatment and vehicle control). The effect of telaglenastat on 2D growth (1 pM-10 μM) over 72 h was measured by MTT (dots−replicates=3), with telaglenastat only exhibiting cytotoxicity at doses >100 nM. Telaglenastat treatment exhibited ˜10% growth in cell number at 100 nM in BME, while it was inactive at 100 nM in 2D.

FIG. 28 is a plot showing representative data in which highly metastatic OS MG63.3 (human) treated with pevonedistat (10-50 nM) lead to appreciable inhibition of 3D growth at doses as low as 10 nM (bars−replicates=4; P<0.005 for all doses ≥10 nM, all comparisons made between treatment and vehicle control). The effect of pevonedistat on 2D growth (3.125-400 nM) over 72 h was measured by MTT (dots−replicates=3), with pevonedistat only exhibiting cytotoxicity at doses >10 nM. Using the lowest active dose ratio index (LADRI), pevonedistat treatment at 25 nM showed increased activity in 3D BME than in 2D, with 20% inhibition in 2D relative to 47% inhibition on 3D leading to a LARDI of 2.35. The 25 nM dose was chosen to determine LARDI as it was the lowest dose in the 2D curve that led to statistically significant inhibition.

FIG. 29 is a plot showing representative data in which highly metastatic OS MG63.3 (human) treated with mitomycin C (12.5-400 nM) lead to appreciable inhibition of 3D growth at doses as low as 12.5 nM (bars−replicates=4; P<0.005 for all doses ≥12.5 nM, all comparisons made between treatment and vehicle control). The effect of mitomycin C on 2D growth (1 pM-10 μM) over 72 h was measured by MTT (dots−replicates=3), with mitomycin C only exhibiting cytotoxicity at doses >200 nM. Mitomycin C treatment was much more potent in 3D BME than in 2D, as shown using the IC50 selectivity index (IC50 SI). With the 3D BME IC50 at −50 nM and the 2D IC50 at ˜1 μM, the IC50 SI for mitomycin C is 20.

FIG. 30A is a plot showing the growth kinetics of matched human (MG63—low; MG63.3—high) osteosarcoma cells as observed over a 6-day period on basement membrane extract. Matched cells were plated under the same conditions and assessed for growth on Days 1, 3 & 6 using an MTS assay. Highly metastatic cells undergo a breakout after 3 days exhibited by the high rate of growth from Day 3-6, while low metastatic lines grew much slower or not at all. These graphs are from a single representative assay (replicates=4), but the experiments were repeated 3 times with similar results.

FIG. 30B is a plot showing the growth kinetics of murine (K12—low; K7M2—high) osteosarcoma cells as observed over a 6-day period on basement membrane extract. Matched cells were plated under the same conditions and assessed for growth on Days 1, 3 & 6 using an MTS assay. Highly metastatic cells undergo a breakout after 3 days exhibited by the high rate of growth from Day 3-6, while low metastatic lines grew much slower or not at all. These graphs are from a single representative assay (replicates=4), but the experiments were repeated 3 times with similar results.

FIG. 31A is a dose response curve as measuring the inhibition of 2D OS cell growth via MTT assay for Regorafenib tested at various doses (10 pM-100 μM) in MG63.3 for 72 h. IC50s were calculated using a four-parameter variable slope nonlinear fit in GraphPad Prism. (N=3, replicates=3-9). Drug exposures below 1 μM were determined to be non-cytotoxic in highly metastatic OS cells.

FIG. 31B is a dose response curve as measuring the inhibition of 2D OS cell growth via MTT assay for Saracatinib tested at various doses (10 pM-100 μM) in MG63.3 for 72 h. IC50s were calculated using a four-parameter variable slope nonlinear fit in GraphPad Prism. (N=3, replicates=3-9). Drug exposures below 1 μM were determined to be non-cytotoxic in highly metastatic OS cells.

FIG. 31C is a dose response curve as measuring the inhibition of 2D OS cell growth via MTT assay for oclacitinib tested at various doses (10 pM-100 μM) in MG63.3 for 72 h. IC50s were calculated using a four-parameter variable slope nonlinear fit in GraphPad Prism. (N=3, replicates=3-9). Drug exposures below 1 μM were determined to be non-cytotoxic in highly metastatic OS cells.

FIG. 31D is a dose response curve as measuring the inhibition of 2D OS cell growth via MTT assay for Regorafenib tested at various doses (10 pM-100 μM) in K7M2 for 72 h. IC50s were calculated using a four-parameter variable slope nonlinear fit in GraphPad Prism. (N=3, replicates=3-9). Drug exposures below 1 μM were determined to be non-cytotoxic in highly metastatic OS cells.

FIG. 31E is a dose response curve as measuring the inhibition of 2D OS cell growth via MTT assay for Saracatinib tested at various doses (10 pM-100 μM) in K7M2 for 72 h. IC50s were calculated using a four-parameter variable slope nonlinear fit in GraphPad Prism. (N=3, replicates=3-9). Drug exposures below 1 μM were determined to be non-cytotoxic in highly metastatic OS cells.

FIG. 31F is a dose response curve as measuring the inhibition of 2D OS cell growth via MTT assay for oclacitinib tested at various doses (10 pM-100 μM) in K7M2 for 72 h. IC50s were calculated using a four-parameter variable slope nonlinear fit in GraphPad Prism. (N=3, replicates=3-9). Drug exposures below 1 μM were determined to be non-cytotoxic in highly metastatic OS cells.

FIG. 32A is a plot showing representative data for Day 6 growth as determined by MTS assay for highly metastatic osteosarcoma cells treated with regorafenib at varying doses (1 nM-10 μM). An in vitro 3D system employing basement membrane extract was used. MG63.3 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 32B is a plot showing representative data for Day 6 growth as determined by MTS assay for highly metastatic osteosarcoma cells treated with saracatinib at varying doses (1 nM-10 μM). An in vitro 3D system employing basement membrane extract was used. MG63.3 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 32C is a plot showing representative data for Day 6 growth as determined by MTS assay for highly metastatic osteosarcoma cells treated with oclacitinib at 100 nM. An in vitro 3D system employing basement membrane extract was used. MG63.3 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 32D is a plot showing representative data for Day 6 growth as determined by MTS assay for highly metastatic osteosarcoma cells treated with oclacitinib at 1 μM. An in vitro 3D system employing basement membrane extract was used. MG63.3 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 32E is a plot showing representative data for Day 6 growth as determined by MTS assay for highly metastatic osteosarcoma cells treated with regorafenib at varying doses (1 nM-10 μM). An in vitro 3D system employing basement membrane extract was used. K7M2 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 32F is a plot showing representative data for Day 6 growth as determined by MTS assay for highly metastatic osteosarcoma cells treated with saracatinib at varying doses (1 nM-10 μM). An in vitro 3D system employing basement membrane extract was used. K7M2 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 32G is a plot showing representative data for Day 6 growth as determined by MTS assay for highly metastatic osteosarcoma cells treated with oclacitinib at varying doses (100 nM and 1 μM). An in vitro 3D system employing basement membrane extract was used. K7M2 cells were treated with the inhibitors and only regorafenib was found to consistently inhibit 3D growth across both cell lines at a dose (1 μM) that is not cytotoxic in 2D. Oclacitinib did not inhibit MG63.3 cells at any dose, while it did lead to a statistically significant but very minimal inhibition in K7M2 cells (N=3, replicates=4; *P<0.05, #P<0.005). The antimetastatic activity of drugs in the 3D BME assay was concordant with findings from human clinical trials, and a novel JAK-1 inhibitor was not in the same assay.

FIG. 33 is a bar graph showing representative data for in vitro 3D BME assay with MG63.3 OS cell line treated with rapamycin (0.5-2 nM), alvespimycin (17.5 nM) or the combination of rapamycin (0.5-2 nM)+alvespimycin (17.5 nM). Highly metastatic OS MG63.3 (human) treated with the combination of rapamycin (0.5-2 nM)+alvespimycin (17.5 nM) led to appreciable inhibition of 3D growth beyond the inhibition exhibited by either drug alone at the same dose (replicates=4; P<0.005 for 0.5-2 nM rapamycin+17.5 nM alvespimycin, all comparisons made between the combination treatment and each drug alone at the same dose).

FIG. 34 is a bar graph showing representative data for in vitro 3D BME assay with K7M2 OS cell line treated with rapamycin (1 nM), alvespimycin (20 nM) or the combination of rapamycin (1 nM)+alvespimycin (20 nM). Highly metastatic OS K7M2 (mouse) treated with the combination of rapamycin (1 nM)+alvespimycin (20 nM) led to inhibition of 3D growth trending beyond the inhibition exhibited by either drug alone at the same dose (replicates=4; P=0.07 for 1 nM rapamycin+20 nM alvespimycin, comparison made between combo and rapamycin alone, P<0.005 for the combination compared to alvespimycin alone).

FIG. 35 is a bar graph showing representative data for in vitro 3D BME assay with MDA-MB-231 highly-metastatic triple-negative breast cancer cell line treated with rapamycin (5 nM), alvespimycin (15-40 nM) or the combination of rapamycin (5 nM)+alvespimycin (15-25 nM). Highly metastatic triple-negative breast cancer (human) treated with the combination of rapamycin (5 nM)+alvespimycin (15-25 nM) led to appreciable inhibition of 3D growth beyond the inhibition exhibited by either drug alone at the same dose (replicates=4; P<0.05 for 5 nM rapamycin+15-25 nM alvespimycin, all comparisons made between the combination treatment and each drug alone at the same dose).

FIG. 36 is a bar graph showing representative data for rapamycin (2 nM), alvespimycin (15-20 nM) or the combination of rapamycin (2 nM)+alvespimycin (15-20 nM) usage on highly metastatic MG63.3 human OS cells in the PuMA assay. Usage of rapamycin (2 nM)+alvespimcyin (20 nM) led to strong inhibition of ME correlating with loss of GFP signal in the lung tissue (% Mean Fluorescent Area) and led to significantly less GFP signal compared to either drug alone (replicates=7; P<0.01 for rapamycin (2 nM)+alvespimycin (20 nM); comparison made between combination and either drug alone).

FIG. 37 is a box and whiskers plot showing representative data for rapamycin (0.5 nM), alvespimycin (25 nM) or the combination of rapamycin (0.5 nM)+alvespimycin (25 nM) usage on highly metastatic HOS-MNNG human OS cells in the PuMA assay. Usage of rapamycin (0.5 nM)+alvespimcyin (25 nM) led to strong inhibition of ME correlating with loss of GFP signal in the lung tissue (Percent Lung Tumor Burden) and led to a trend of less GFP signal compared to either drug alone (replicates=7; P<0.05 for rapamycin (2 nM)+alvespimycin (20 nM) compared to rapamycin alone, P=0.08 for the combination compared to alvespimycin alone.

FIG. 38 is a bar graph showing representative data for in vitro 3D BME assay with MG63.3 OS cell line treated with rapamycin (0.5 nM), domatinostat (100-400 nM) or the combination of rapamycin (0.5 nM)+domatinostat (100-300 nM). Highly metastatic OS MG63.3 (human) treated with the combination of rapamycin (0.5 nM)+domatinostat (200 & 300 nM) led to appreciable inhibition of 3D growth beyond the inhibition exhibited by either drug alone at the same dose (replicates=4; P<0.005 for 0.5 nM rapamycin+200/300 nM domatinostat, all comparisons made between the combination treatment and each drug alone at the same dose).

FIG. 39 is a box and whiskers plot showing representative data for rapamycin (1 nM), domatinostat (100-300 nM) or the combination of rapamycin (1 nM)+domatinostat (100 nM) usage on highly metastatic MG63.3 human OS cells in the PuMA assay. Usage of rapamycin (1 nM)+domatinostat (100 nM) led to strong inhibition of ME correlating with loss of GFP signal in the lung tissue (Percent Lung Tumor Burden) and led to significantly less GFP signal compared to either drug alone (replicates=7; P<0.005 for rapamycin (1 nM)+domatinostat (100 nM); comparison made between combination and either drug alone).

FIG. 40 is a bar graph showing representative data for in vitro 3D BME assay with MG63.3 OS cell line treated with rapamycin (1 nM), domatinostat (100 & 150 nM) or the combination of rapamycin (1 nM)+domatinostat (100 & 150 nM) treated once, as well as all the same conditions treated once, followed by a re-treatment on Day 3. Highly metastatic OS MG63.3 (human) re-treated with rapamycin, domatinostat and the combination led to significantly increased inhibition relative to the conditions only treated with drug once, except for 100 nM domatinostat alone (P<0.01 for all conditions except 100 nM domatinostat alone; all comparisons made between the same conditions either treated once or re-treated). The combination of rapamycin (1 nM)+domatinostat (100/150 nM) led to appreciable inhibition of 3D growth beyond the inhibition exhibited by either drug alone at the same dose with re-treated conditions (replicates=4; P<0.05 for 1 nM rapamycin+100/150 nM domatinostat re-treated, all comparisons made between the combination treatment and each drug alone at the same dose).

FIG. 41 is a bar graph showing representative data for in vitro 3D BME assay with MDA-MB-231 highly-metastatic triple-negative breast cancer cell line treated with rapamycin (5 nM), domatinostat (100-300 nM) or the combination of rapamycin (5 nM)+domatinostat (100 & 150 nM). Highly metastatic MDA-MB-231 triple-negative breast cancer (human) treated with the combination of rapamycin (5 nM)+domatinostat (100 & 150 nM) led to appreciable inhibition of 3D growth beyond the inhibition exhibited by either drug alone at the same dose (replicates=4; P<0.05 for 0.5 nM rapamycin+100/150 nM domatinostat, all comparisons made between the combination treatment and each drug alone at the same dose).

FIG. 42 is a bar graph showing representative data for in vitro 3D BME assay with SK-MEL-28 highly metastatic melanoma cell line treated with rapamycin (5-20 nM), domatinostat (100-300 nM) or the combination of rapamycin (10 nM)+domatinostat (100 & 200 nM). Highly metastatic SK-MEL-28 melanoma (human) treated with the combination of rapamycin (10 nM)+domatinostat (200 nM) led to appreciable inhibition of 3D growth beyond the inhibition exhibited by either drug alone at the same dose (replicates=4; P<0.05 for 10 nM rapamycin+200 nM domatinostat, all comparisons made between the combination treatment and each drug alone at the same dose).

FIG. 43 is a bar graph showing representative data for in vitro 3D BME assay with MG63.3 OS cell line treated with rapamycin (0.5 nM), navitoclax (1000 nM) or the combination of rapamycin (0.5 nM)+navitoclax (1000 nM). Highly metastatic OS MG63.3 (human) treated with the combination of rapamycin (0.5 nM)+navitoclax (1000 nM) led to appreciable inhibition of 3D growth beyond the inhibition exhibited by either drug alone at the same dose (replicates=4; P<0.005 for 0.5 nM rapamycin+1000 nM navitoclax, all comparisons made between the combination treatment and each drug alone at the same dose).

FIG. 44 is a box and whiskers plot showing representative data for rapamycin (2 nM), daporinad (2-3 nM) or the combination of rapamycin (2 nM)+daporinad (2 nM) usage on highly metastatic MG63.3 human OS cells in the PuMA assay. Usage of rapamycin (2 nM)+daporinad (2 nM) led to strong inhibition of ME correlating with loss of GFP signal in the lung tissue (% Mean Fluorescent Area) and led to significantly less GFP signal compared to either drug alone (replicates=7; P<0.005 for rapamycin (2 nM)+daporinad (2 nM) compared to rapamycin alone, P<0.05 for the combination compared to daporinad alone.

FIG. 45 is a box and whiskers plot showing representative data for rapamycin (0.1 nM), daporinad (0.5 nM) or the combination of rapamycin (0.1 nM)+daporinad (0.5 nM) usage on highly metastatic HOS-MNNG human OS cells in the PuMA assay. Usage of rapamycin (0.1 nM)+daporinad (0.5 nM) led to strong inhibition of ME correlating with loss of GFP signal in the lung tissue (Percent Lung Tumor Burden) and led to a trend of less GFP signal compared to either drug alone (replicates=7; P=0.06 for rapamycin (0.1 nM)+daporinad (0.5 nM) compared to rapamycin alone, P=0.12 for the combination compared to daporinad alone.

FIG. 46 is a bar graph showing representative data for in vitro 3D BME assay with MG63.3 OS cell line treated with rapamycin (0.5-1 nM), PD0325901 (1-10 nM) or the combination of rapamycin (0.5-1 nM)+PD0325901 (1-10 nM). Highly metastatic OS MG63.3 (human) treated with the combination of rapamycin (0.5 nM)+PD0325901 (10 nM) led to appreciable inhibition of 3D growth beyond the inhibition exhibited by either drug alone at the same dose (replicates=4; P<0.005 for 0.5 nM rapamycin+10 nM PD0325901, all comparisons made between the combination treatment and each drug alone at the same dose).

FIG. 47 is a bar graph showing representative data for in vitro 3D BME assay with MDA-MB-231 highly metastatic triple-negative breast cancer cell line treated with rapamycin (5 nM), PD0325901 (3.2-80 nM) or the combination of rapamycin (5 nM)+PD0325901 (3.2-80 nM). Highly metastatic triple-negative breast cancer cells MDA-MB-231 (human) treated with the combination of rapamycin (5 nM)+PD0325901 (3.2 nM) led to appreciable inhibition of 3D growth beyond the inhibition exhibited by either drug alone at the same dose (replicates=4; P<0.01 for 0.5 nM rapamycin+3.2 nM PD0325901, all comparisons made between the combination treatment and each drug alone at the same dose).

FIG. 48 is a box and whiskers plot showing representative data for rapamycin (1-2 nM), PD0325901 (10 nM) or the combination of rapamycin (1 nM)+PD0325901 (10 nM) usage on highly metastatic MG63.3 human OS cells in the PuMA assay. Usage of rapamycin (1 nM)+PD0325901 (10 nM) led to strong inhibition of ME correlating with loss of GFP signal in the lung tissue (Percent Lung Tumor Burden) and led to significantly less GFP signal compared to either drug alone (replicates=7; P<0.05 for rapamycin (1 nM)+PD0325901 (10 nM); comparison made between combination and either drug alone.

FIG. 49 is a bar graph showing representative data for in vitro 3D BME assay with MG63.3 OS cell line treated with alvespimycin (10-20 nM), daporinad (0.25-0.5 nM) or the combination of alvespimycin (10-20 nM)+daporinad (0.25-0.5 nM). Highly metastatic OS MG63.3 (human) treated with the combination of alvespimycin (15 & 20 nM)+daporinad (0.25 nM) led to appreciable inhibition of 3D growth beyond the inhibition exhibited by either drug alone at the same dose (replicates=4; P<0.005 for alvespimycin (15 & 20 nM)+daporinad (0.25 nM), all comparisons made between the combination treatment and each drug alone at the same dose).

FIG. 50 is a bar graph showing representative data for in vitro 3D BME assay with MG63.3 OS cell line treated with alvespimycin (20 nM), domatinostat (100 & 200 nM) or the combination of alvespimycin (20 nM)+domatinostat (100 & 200 nM). Highly metastatic OS MG63.3 (human) treated with the combination of alvespimycin (20 nM)+domatinostat (200 nM) led to appreciable inhibition of 3D growth beyond the inhibition exhibited by either drug alone at the same dose (replicates=4; P<0.005 for alvespimycin (20 nM)+domatinostat (200 nM), all comparisons made between the combination treatment and each drug alone at the same dose).

FIG. 51 is a bar graph showing representative data for in vitro 3D BME assay with MG63.3 OS cell line treated with navitoclax (500 nM), domatinostat (100 nM) or the combination of navitoclax (500 nM)+domatinostat (100 nM). Highly metastatic OS MG63.3 (human) treated with the combination of navitoclax (500 nM)+domatinostat (100 nM) led to appreciable inhibition of 3D growth beyond the inhibition exhibited by either drug alone at the same dose (replicates=4; P<0.05 for navitoclax (500 nM)+domatinostat (100 nM), all comparisons made between the combination treatment and each drug alone at the same dose).

FIG. 52 is a box and whiskers plot showing representative data for domatinostat (150-400 nM), navitoclax (500 nM) or the combination of domatinostat (200 nM)+navitoclax (500 nM) usage on highly metastatic HOS-MNNG human OS cells in the PuMA assay. Usage of domatinostat (200 nM)+navitoclax (500 nM) led to strong inhibition of ME correlating with loss of GFP signal in the lung tissue (Percent Lung Tumor Burden) and led to a trend of less GFP signal compared to either drug alone (replicates=7; P<0.005 for domatinostat (200 nM)+navitoclax (500 nM) relative to navitoclax alone, P=0.13 relative to domatinostat alone.

FIG. 53 is a bar graph showing representative data for in vitro 3D BME assay with MG63.3 OS cell line treated with daporinad (0.25 & 0.5 nM), domatinostat (150 & 200 nM) or the combination of daporinad (0.25 & 0.5 nM)+domatinostat (150 & 200 nM). Highly metastatic OS MG63.3 (human) treated with the combination of daporinad (0.25 & 0.5 nM)+domatinostat (150 & 200 nM) led to appreciable inhibition of 3D growth beyond the inhibition exhibited by either drug alone at the same doses (replicates=4; P<0.01 for all combinations of daporinad (0.25 & 0.5 nM)+domatinostat (150 & 200 nM), all comparisons made between the combination treatment and each drug alone at the same dose).

FIG. 54 is a bar graph showing representative data for in vitro 3D BME assay with MG63.3 OS cell line treated with rapamycin (1 nM), mivebresib (10-30 nM) or the combination of rapamycin (1 nM)+mivebresib (10 & 20 nM). Highly metastatic OS MG63.3 (human) treated with the combination of rapamycin (1 nM)+mivebresib (10 & 20 nM) led to appreciable inhibition of 3D growth beyond the inhibition exhibited by either drug alone at the same doses (replicates=4; P<0.05 for all combinations of rapamycin (1 nM)+mivebresib (10 & 20 nM), all comparisons made between the combination treatment and each drug alone at the same dose).

FIG. 55 is a bar graph showing representative data for in vitro 3D BME assay with MDA-MB-231 highly metastatic triple-negative breast cancer cell line treated with rapamycin (5 nM), mivebresib (2.5 & 5 nM) or the combination of rapamycin (5 nM)+mivebresib (2.5 & 5 nM). Highly metastatic triple-negative breast cancer cells (human) treated with the combination of rapamycin (5 nM)+mivebresib (2.5 & 5 nM) led to appreciable inhibition of 3D growth beyond the inhibition exhibited by either drug alone at the same doses (replicates=4; P<0.005 for all combinations of rapamycin (5 nM)+mivebresib (2.5 & 5 nM), all comparisons made between the combination treatment and each drug alone at the same dose).

FIG. 56 is a bar graph showing representative data for in vitro 3D BME assay with MG63.3 OS cell line treated with daporinad (0.25 & 0.5 nM), domatinostat (150 & 200 nM) or the combination of daporinad (0.25 & 0.5 nM)+domatinostat (150 & 200 nM). Highly metastatic OS MG63.3 (human) treated with the combination of daporinad (0.25 & 0.5 nM)+domatinostat (150 & 200 nM) led to appreciable inhibition of 3D growth beyond the inhibition exhibited by either drug alone at the same doses (replicates=4; P<0.01 for all combinations of daporinad (0.25 & 0.5 nM)+domatinostat (150 & 200 nM), all comparisons made between the combination treatment and each drug alone at the same dose).

FIG. 57 is a bar graph showing representative data for in vitro 3D BME assay with MG63.3 OS cell line treated with mivebresib (10 nM), domatinostat (100 nM) or the combination of mivebresib (10 nM)+domatinostat (100 nM). Highly metastatic OS MG63.3 (human) treated with the combination of mivebresib (10 nM)+domatinostat (100 nM) led to appreciable inhibition of 3D growth beyond the inhibition exhibited by either drug alone at the same doses (replicates=4; P<0.01 for all combinations of mivebresib (10 nM)+domatinostat (100 nM), all comparisons made between the combination treatment and each drug alone at the same dose).

FIG. 58 is a scatterplot showing representative data for in vitro 3D BME assay with MG63.3 OS cell line treated with various doses of HDAC inhibitors. The activity in 3D BME [%] of MG63.3 growth is plotted against the Selectivity for ME, which is a ratio of 3D activity to 2D activity (% inhibition) at the same dose. The doses were chosen based off of dose-response data, with the corresponding dose leading to the best selectivity/activity profile chosen. Compounds with higher selectivity and activity cluster in the upper right, and it is interesting to note that many Class I-selective HDAC inhibitors (black filled symbols) afforded the highest inhibition & selectivity while all of the pan-HDAC inhibitors (grey filled symbols) had lower selectivity/activity.

FIG. 59 is a graph showing representative data for in vitro 3D BME assay (bar) and 2D assay (line) with MG63.3 OS cell line with domatinostat. Highly metastatic OS MG63.3 (human) treated with domatinostat (100-400 nM) led to appreciable inhibition of 3D growth at doses as low as 100 nM (bars−replicates=4; P<0.005 for all doses ≥100 nM, all comparisons made between treatment and vehicle control). The effect of domatinostat on 2D growth (100-400 nM) over 72 h was measured by MTT (dots−replicates=3), with domatinostat exhibiting cytotoxicity at doses ≥200 nM. However, domatinostat treatment exhibited much stronger activity on 3D BME at similar doses.

FIG. 60 is a graph showing representative data for in vitro 3D BME assay (bar) and 2D assay (line) with MG63.3 OS cell line with navitoclax. Highly metastatic OS MG63.3 (human) treated with navitoclax (500-2000 nM) led to appreciable inhibition of 3D growth at doses as low as 500 nM (bars−replicates=4; P<0.005 for all doses ≥500 nM, all comparisons made between treatment and vehicle control). The effect of navitoclax on 2D growth (1 pM-10 μM) over 72 h was measured by MTT (dots−replicates=3), with navitoclax exhibiting cytotoxicity at doses ≥1 μM. However, navitoclax treatment exhibited much stronger activity on 3D BME at similar doses.

FIG. 61 is a graph showing representative data for in vitro 3D BME assay (bar) and 2D assay (line) with MG63.3 OS cell line with PD0325901. Highly metastatic OS MG63.3 (human) treated with PD0325901 (1-128 nM) led to appreciable inhibition of 3D growth at doses as low as 1 nM (bars−replicates=4; P<0.005 for all doses ≥1 nM, all comparisons made between treatment and vehicle control). The effect of PD0325901 on 2D growth (1 pM-10 μM) over 72 h was measured by MTT (dots−replicates=3), with PD0325901 exhibiting cytotoxicity at doses ≥100 nM. However, PD0325901 treatment exhibited much stronger activity on 3D BME at similar doses.

FIG. 62 is a graph showing representative data for in vitro 3D BME assay (bar) and 2D assay (line) with MG63.3 OS cell line with belinostat. Highly metastatic OS MG63.3 (human) treated with belinostat (100-300 nM) led to appreciable inhibition of 3D growth at doses as low as 200 nM (bars−replicates=4; P<0.005 for all doses ≥200 nM, all comparisons made between treatment and vehicle control). The effect of belinostat on 2D growth (100 pM-10 μM) over 72 h was measured by MTT (dots−replicates=3), with belinostat exhibiting cytotoxicity at doses ≥80 nM. However, belinostat treatment exhibited modestly stronger activity on 3D BME at 200 nM and above.

FIG. 63 is a graph showing representative data for in vitro 3D BME assay (bar) and 2D assay (line) with MG63.3 OS cell line with panobinostat. Highly metastatic OS MG63.3 (human) treated with panobinostat (40-80 nM) led to appreciable inhibition of 3D growth at doses as low as 40 nM (bars−replicates=4; P<0.005 for all doses ≥40 nM, all comparisons made between treatment and vehicle control). The effect of panobinostat on 2D growth (100 pM-10 μM) over 72 h was measured by MTT (dots−replicates=3), with panobinostat exhibiting cytotoxicity at doses ≥80 nM. However, panobinostat treatment exhibited slightly stronger activity on 3D BME at 60 nM and above.

FIG. 64 is a graph showing representative data for in vitro 3D BME assay (bar) and 2D assay (line) with MG63.3 OS cell line with givinostat. Highly metastatic OS MG63.3 (human) treated with givinostat (300-500 nM) led to appreciable inhibition of 3D growth at doses as low as 300 nM (bars−replicates=4; P<0.005 for all doses ≥300 nM, all comparisons made between treatment and vehicle control). The effect of givinostat on 2D growth (100 nM-1400 nM) over 72 h was measured by MTT (dots−replicates=3), with givinostat exhibiting cytotoxicity at doses ≥600 nM. However, givinostat treatment exhibited much stronger activity on 3D BME at 300 nM and above.

FIG. 65 is a graph showing representative data for in vitro 3D BME assay (bar) and 2D assay (line) with MG63.3 OS cell line with quisinostat. Highly metastatic OS MG63.3 (human) treated with quisinostat (10-80 nM) led to appreciable inhibition of 3D growth at doses as low as 10 nM (bars−replicates=4; P<0.005 for all doses ≥10 nM, all comparisons made between treatment and vehicle control). The effect of quisinostat on 2D growth (100 pM-10 μM) over 72 h was measured by MTT (dots−replicates=3), with quisinostat exhibiting cytotoxicity at doses ≥80 nM. However, quisinostat treatment exhibited much stronger activity on 3D BME at 10 nM and above.

FIG. 66 is a graph showing representative data for in vitro 3D BME assay (bar) and 2D assay (line) with MG63.3 OS cell line with fimepinostat. Highly metastatic OS MG63.3 (human) treated with fimepinostat (5-10 nM) led to complete inhibition of 3D growth at doses as low as 5 nM (bars−replicates=4; P<0.005 for all doses ≥5 nM, all comparisons made between treatment and vehicle control). The effect of fimepinostat on 2D growth (100 pM-10 μM) over 72 h was measured by MTT (dots−replicates=3), with fimepinostat exhibiting cytotoxicity at doses ≥1 nM. However, fimepinostat treatment exhibited much stronger activity on 3D BME at 5 nM and above.

FIG. 67 is a bar graph showing representative data for in vitro 3D BME assay with MG63.3 OS cell line with domatinostat (200-800 nM) treated on Days 3-6 (2 days off, 4 days on) or treated on Days 1-2 (2 days on, 4 days off). Highly metastatic OS MG63.3 (human) treated with domatinostat led to significantly inhibition relative to vehicle for both treatment schedules (P<0.005 for all conditions; all comparisons made between treatment and vehicle control). The earlier treatment with domatinostat (2 days on, 4 days off) led to slightly more inhibition than later treatment (2 days off, 4 days on), despite the latter condition being treated for a longer time.

FIG. 68 is a bar graph showing representative data for in vitro 3D BME assay with MG63.3 OS cell line with rapamycin (2-16 nM) treated for 6 days (6 days on), on Days 3-6 (2 days off, 4 days on) or treated on Days 1-2 (2 days on, 4 days off). Highly metastatic OS MG63.3 (human) treated with rapamycin led to significantly inhibition relative to vehicle for both treatment schedules (P<0.005 for all conditions; all comparisons made between treatment and vehicle control). The earlier treatment with domatinostat (2 days on, 4 days off) led to slightly more inhibition than later treatment (2 days off, 4 days on), despite the latter condition being treated for a longer time. Treatment for 6 days (6 days on) led to the strongest inhibition from 2-4 nM, but was fairly similar to earlier treatment (2 days on, 4 days off).

FIG. 69 is a bar graph showing representative data for in vitro 3D BME assay with MG63.3 OS cell line treated with rapamycin (1-2 nM—only on Day 1—1 day on, 5 days off), domatinostat (100-200 nM—only on Day 1—1 day on, 5 days off) or the combination of rapamycin (1-2 nM)+domatinostat (100-200 nM) only on Day 1 (1 day on, 5 days off). Highly metastatic OS MG63.3 (human) treated on Day 1 with the combination of domatinostat+rapamycin led to significantly increased inhibition relative to either drug alone, except for 2 nM rapamycin+100 nM domatinostat (P<0.05 for all combinations relative to domatinostat and P<0.01 for all combinations relative to rapamycin alone except for 2 nM rapamycin+100 nM domatinostat). The combination of rapamycin+domatinostat treated for only 1 day led to appreciable inhibition of 3D growth beyond the inhibition exhibited by either drug alone treated for 1 day.

FIG. 70 is a bar graph showing representative data for in vitro 3D BME assay with MG63.3 OS cell line treated with rapamycin (1-2 nM—6 days—6 days on), domatinostat (100-200 nM—1 day only—1 day on, 5 days off) or the combination of rapamycin (1-2 nM)+domatinostat (100-200 nM) treated with the combination for 1 day, followed by removal of drug and re-treated with rapamycin only for 5 days (24 h combo, 5 days rapamycin only). Highly metastatic OS MG63.3 (human) re-treated with rapamycin, after treatment with the combination of domatinostat+rapamycin for 1 day led to significantly increased inhibition relative to the conditions treated with either rapamycin alone for 6 days (6 days on) or domatinostat alone for 1 day only (1 day on, 5 days off) (P<0.005 for all combinations; all comparisons made between the combinations and the related single agent treatment). This combination showed strong activity despite only being treated with domatinostat for 1 day, which had little to no efficacy at these doses for 1 day domatinostat alone treatment.

FIG. 71 is a bar graph showing representative data for in vitro 3D BME assay with MG63.3 OS cell line treated with rapamycin (1-2 nM—1 day only—1 day on, 5 days off), domatinostat (100-200 nM—6 days—6 days on) or the combination of rapamycin (1-2 nM)+domatinostat (100-200 nM) treated with the combination for 1 day, followed by removal of drug and re-treated with domatinostat only for 5 days (24 h combo, 5 days domatinostat only). Highly metastatic OS MG63.3 (human) re-treated with domatinostat, after treatment with the combination of domatinostat+rapamycin for 1 day, led to significantly increased inhibition relative to the conditions treated with either domatinostat alone for 6 days (6 days on) or rapamycin alone for 1 day only (1 day on, 5 days off) (P<0.005 for all combinations; all comparisons made between the combinations and the related single agent treatment). This combination showed strong activity despite only being treated with rapamycin for 1 day, which had only modest efficacy at these doses for 1 day rapamycin alone treatment.

FIG. 72 is a box and whiskers plot showing representative data for the combination of domatinostat (300 nM)+rapamycin (2 nM) treated for 7 days (7 days on, 7 days off) usage on highly metastatic HOS-MNNG human OS cells in the PuMA assay. Usage of rapamycin (2 nM)+domatinostat (300 nM) led to near complete inhibition of ME correlating with loss of GFP signal in the lung tissue (Percent Lung Tumor Burden) and led to significantly less GFP signal compared to vehicle (replicates=7; P<0.005 for the combination relative to vehicle). Treating the lung slices with the combination for only the 1^{st} 7 days of the 14 day experiment still led to extremely strong inhibition.

FIG. 73 is a graph showing representative data for in vitro 3D BME assay (bar) and 2D assay (line) with MG63.3 OS cell line with halofuginone. Highly metastatic OS MG63.3 (human) treated with halofuginone (0.75-100 nM) led to >25% inhibition of 3D growth at doses as low as 3 nM (bars−replicates=4; P<0.005 for all doses ≥3 nM, all comparisons made between treatment and vehicle control). The effect of halofuginone on 2D growth (781 pM-128 nM) over 72 h was measured by MTT (dots−replicates=3), with halofuginone exhibiting cytotoxicity at doses ≥12.5 nM. However, halofuginone treatment exhibited stronger activity on 3D BME at 3 nM and above.

FIG. 74 is a graph showing representative data for in vitro 3D BME assay (bar) and 2D assay (line) with MG63.3 OS cell line with minnelide. Highly metastatic OS MG63.3 (human) treated with minnelide (0.25-64 nM) led to complete inhibition of 3D growth at doses as low as 4 nM (bars−replicates=4; P<0.005 for all doses ≥4 nM, all comparisons made between treatment and vehicle control). The effect of minnelide on 2D growth (1 pM-10 μM) over 72 h was measured by MTT (dots−replicates=3), with minnelide exhibiting cytotoxicity at doses ≥781 pM. However, minnelide treatment exhibited stronger activity on 3D BME at 2 nM and above.

FIG. 75 is a graph showing representative data for in vitro 3D BME assay (bar) and 2D assay (line) with MG63.3 OS cell line with triptolide. Highly metastatic OS MG63.3 (human) treated with triptolide (0.25-1 nM) led to complete inhibition of 3D growth at doses as low as 1 nM (bars−replicates=4; P<0.005 for all doses, all comparisons made between treatment and vehicle control). The effect of triptolide on 2D growth (1 pM-10 μM) over 72 h was measured by MTT (dots−replicates=3), with triptolide exhibiting cytotoxicity at all doses tested. However, triptolide treatment exhibited stronger activity on 3D BME at 0.5 nM and above.

FIG. 76 is a graph showing representative data for in vitro 3D BME assay (bar) and 2D assay (line) with MG63.3 OS cell line with pimasertib. Highly metastatic OS MG63.3 (human) treated with pimasertib (100-1600 nM) led to complete inhibition of 3D growth at doses as low as 100 nM (bars−replicates=4; P<0.005 for all doses, all comparisons made between treatment and vehicle control). The effect of pimasertib on 2D growth (25.6 pM-10 μM) over 72 h was measured by MTT (dots−replicates=3), with pimasertib exhibiting cytotoxicity at all doses above 1 nM. However, pimasertib treatment exhibited stronger activity on 3D BME at 100 nM and above.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present disclosure includes compositions of matter capable of targeting, acting on and/or disabling or weakening and/or attacking dormant cancer cells. These compositions may also cause the patient's body to disable and/or attack dormant cancer cells. It includes the use of novel and existing compositions, the design, discovery optimization of novel and existing therapeutic agents, novel formulations, dosing, delivery and administration pharmaceutical products capable of targeting and attacking dormant cancer cells.

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “an antidepressant,” “a psychological disorder,” or “a subject” includes mixtures of two or more such antidepressants, psychological disorders, or subjects, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “dormant” means a quiescent state (a reversible state of a cell in which it does not divide but retains the ability to re-enter cell proliferation), a state where cellular proliferation is balanced by cell death or apoptosis, a stem cell-like state, a suppressed state, or other state in which cancer cells can-not rapidly divide or begin to rapidly divide. “Dormant cancer cells” or “DTCs” refers to disseminated dormant, micrometastatic, or suppressed living cells or cell clusters which have been disseminated from a primary tumor or from secondary tumors that are not expressing their malignant phenotype and/or cells that are in a stage in cancer progression where the cells cease dividing but survive in a quiescent state while waiting for appropriate environmental conditions to begin proliferation again, and/or the cells are not dividing but at arrest in the cell cycle in G₀-G₁. Dormant DTCs may reside as single solitary quiescent cells (cellular dormancy) and/or as small clusters of quiescent cells. Others may reside as small indolent micrometastases where cellular proliferation is balanced by apoptosis.

They can be present in early tumor progression, in micrometastases, or left behind in minimal residual disease (MRD) after what was thought to be a successful treatment of the primary tumor.

Dormant cancer cells may be present in many organs and sites in the body including but not limited to organs and tissues such as the lungs, liver, brain, bone, bone marrow, intestines, skin, muscles and vagina, to lymph nodes and to glands such as the adrenal glands, and cavities such as the abdominal (peritoneal) cavity.

Dormant cancer cells may be present long enough to be controlled by suitable therapeutic interventions in order to prevent, delay or reduce cancer recurrence. Such interventions can exhibit one or more of a number of intervention modes. They may:
Target and inhibit intrinsic mechanisms that cause dormant cancer cells to exit from dormancy.
Maintain intrinsic mechanisms that keep dormant cancer cells dormant.
Disable or weaken the Metastatic Endurance of dormant cancer cells.
Target the interaction between dormant cancer cells and their local microenvironment that control dormancy/dormancy exit.
Cause targeted killing of dormant cancer cells.
Cause targeted killing of dormant cancer cells exiting dormancy.
Inhibit dormant cancer cells exiting dormancy form switching to rapid proliferation.
Effect the immune system to target dormant cells.

As used herein, the term “microscopic metastatic tumor” means one cell or clusters of few or many cells, including persistent cancer cells, persistent disseminated tumor cells, dormant cancer cells, micrometastatic cancer cells, suppressed cancer cells, and micrometastatic tumors. A micrometastatic tumor may be composed of various cancer cells or cell states such as quiescent, suppressed, rapidly dividing cells and other cells with an overall lack of, or small change in growth or cell number over time. Single cells, multiple cells, or a portion of the cells in a microscopic metastatic tumor may be in a quiescent state (a reversible state of a cell in which it does not divide but retains the ability to re-enter cell proliferation), a state where cellular proliferation is balanced by cell death or apoptosis, a stem cell-like state, a suppressed state, or other state in which cancer cells can-not rapidly divide or begin to rapidly divide, or in which overall tumor size doesn't change or changes slowly. These cancer cells described above can be disseminated from a primary tumor or from secondary tumors and/or cells that are in a stage in cancer progression where the cells cease dividing but survive in a quiescent state while waiting for appropriate environmental conditions to begin proliferation again, and/or the cells are not dividing but at arrest in the cell cycle in G₀-G₁. These cancer cells may reside as single solitary quiescent cells (e.g., cellular dormancy) and/or as small clusters of quiescent cells. Cells of a microscopic metastatic tumor may reside as small indolent micrometastases where cellular proliferation is balanced by apoptosis or other means such as killing by the immune system or other, in which the result is no growth or slow growing micrometastases.

A microscopic metastatic tumor can include one cell or clusters of few or many cells, including persistent cancer cells, persistent disseminated tumor cells, micrometastatic cancer cells, and suppressed cancer cells. As would be understood by one of ordinary skill, a microscopic metastatic tumor can be composed of various cancer cells or cell states such as quiescent, suppressed, rapidly dividing cells, and other cells with an overall lack of or small change in growth or cell number over time. Single cells, multiple cells, or a portion of the cells in a microscopic metastatic tumor may be in a quiescent state (a reversible state of a cell in which it does not divide but retains the ability to re-enter cell proliferation), a state where cellular proliferation is balanced by cell death or apoptosis, a stem cell-like state, a suppressed state, or some other state in which cancer cells cannot rapidly divide or begin to rapidly divide or in which overall tumor size doesn't change or changes slowly. Without wishing to be bound by theory, these cancer cells can be disseminated from a primary tumor, a secondary tumor, and/or cells that are in a stage in cancer progression where the cells cease dividing but survive in a quiescent state while waiting for appropriate environmental conditions to begin proliferation again and/or where the cells are not dividing but are at arrest in the cell cycle in G0-G1. These cancer cells may reside as single solitary quiescent cells (e.g., cellular dormancy) and/or as small clusters of quiescent cells. Cells of a microscopic metastatic tumor may reside as small indolent micrometastases where cellular proliferation is balanced by apoptosis or other means such as killing by the immune system or other means in which the result is no growth or slow growing micrometastases.

Microscopic metastatic tumors can be present in early tumor progression, in micrometastases, or left behind in minimal residual disease (MRD) after what was thought to be a successful treatment of the primary tumor or of initial or subsequent metastatic tumors using surgery, radiation, drug treatment of any kind (including as examples small molecules, biologics and cell therapies amongst other interventions), or the combination of some or all of these treatments.

“Metastatic Endurance” (or “ME”) as used herein refers to the combination of any one or more of stress adaption, survival or dormancy that is characteristic of DTCs and/or microscopic metastatic tumors that can successfully reside at secondary metastatic sites and eventually form overt metastases. Independent of or as part of the process of dormancy, no growth or slow growth, a requirement of successful DTCs and microscopic metastatic tumors is to adapt and survive a variety of stressors experienced in the new microenvironment of the secondary site. It is thought that the stress conditions encountered upon arrival, may instigate early stress adaptation mechanisms and programs, later stage stress adaptations and long-term survival that results in dormancy, no growth or slow growth, and overall metastatic success.

Microscopic metastatic tumors may be present in many organs and sites in the body including but not limited to organs and tissues such as the lungs, liver, brain, bone, bone marrow, intestines, skin, muscles and vagina, to lymph nodes and to glands such as the adrenal glands, and cavities such as the abdominal (peritoneal) cavity.

Microscopic metastatic tumors may be present long enough to be controlled by suitable therapeutic interventions in order to prevent, delay or reduce initial cancer recurrence and/or subsequent cancer recurrences. Such interventions can exhibit one or more of a number of intervention modes. They may:
Target and inhibit intrinsic mechanisms that cause microscopic metastatic tumors to exit from dormancy;
Maintain intrinsic mechanisms that keep microscopic metastatic tumors in a dormant, no growth or slow growth state;
Disable or weaken the Metastatic Endurance of microscopic metastatic tumors;
Target the interaction between microscopic metastatic tumor and their local microenvironment that control dormancy/dormancy exit and/or slow growth state;
Cause targeted killing of microscopic metastatic tumors;
Cause targeted killing of microscopic metastatic tumors exiting dormancy or slow growth state;
Inhibit microscopic metastatic tumor exiting dormancy or no/slow growth state form switching to rapid proliferation and/or growth; and/or
Effect the immune system to target microscopic metastatic tumors.

A “metastatic endurance phenotype” as used herein refers to any observable set of cellular features in which a cell is expressing a metastatic endurance. This can be caused by various underlying genetic, epigenetic, post-translational, immune-related, structural and other yet-to-be-discovered biological mechanisms that enable this phenotype. Such phenotype can be assessed by various methods, including by using various cell biology and biochemistry techniques to determine that cancer cells are in growth kinetics in which cells are quiescent, slow-dividing, suppressed, at a steady-state between cell death or apoptosis, and division, not changing in number or have a reduced ability to proliferate for a period of time, prior to a period of rapid growth. This can also be assessed by methods of the invention, including administering a compound known to be active against ME, as will be described in various embodiments of this invention.

The term “controlling” is intended to refer to all processes wherein there may be a slowing, delaying, suppressing, interrupting, arresting, and/or stopping of the progression of the disease and does not necessarily indicate a total elimination of all disease symptoms.

The term “target” is intended to refer to all processes wherein there may be targeting, disabling, inhibiting, modifying, enhancing, upregulating, acting as an antagonist or as an agonist or both, killing, delaying, suppressing, interrupting, arresting, and/or stopping a function of a cell.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “therapeutically effective amount” is further meant to define an amount resulting in the improvement of any parameters or clinical symptoms characteristic of cancer. It is further mean to define and amount that may aid in prevention and controlling cancer progression. The actual dose will be different for the various specific molecules, and will vary with the patient's overall condition, the seriousness of the symptoms, and counter indications.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

“Subject” means any living subject, including mammalian subjects such as a human. The subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

“Therapeutic agent” refers to any agent capable of producing the relevant biological effect, including small molecule compounds, larger compounds, biologics, monoclonal antibodies, peptides, amino acids, proteins, among other examples. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions, which can be dissolved or dispersed in sterile water or other sterile injectable media just prior to use. Suitable inert carriers can include sugars such as lactose.

As used herein, the term “substantially similar” refers to something that is to a great extent or essentially the same. This may refer to experimental subjects that are treated in a near-identical fashion in order to more accurately make comparisons between experimental groups. A non-limiting example would be to measure a value from two different samples at as close a time as possible, so as to obtain values from substantially similar times.

As used herein, the term “selective activity” means an effect that is stronger for one phenotype over another. This can include any activity above zero, which can be precisely measured above the limit of detection for a given assay. For a non-limiting example, a drug may possess selective activity against ME if it has a greater level of inhibition of highly metastatic cancer cells grown in 3D ME-supporting conditions than the level of inhibition of the same cells grown in 2D rapidly-dividing conditions.

As used herein, the term “specific activity” means an effect only occurs in phenotype over another. Specific activity can be observed when something has 100% activity in one condition, while no impact in another condition. For a non-limiting example, a drug may possess specific activity against ME if it has a complete inhibition of highly metastatic cancer cells grown in 3D ME-supporting conditions while there is no effect observed on the same cells grown in 2D rapidly-dividing conditions.

As used herein, the term “approximately” means that the amount or value in question can be the value designated some other value is about the same. It is generally understood, as used herein, that it is the nominal value indicated ±10% variation unless otherwise indicated or inferred.

As used herein, the term “partial remission” means a decrease in the size of a tumor, or in the extent of cancer in the body, in response to treatment. Also called partial response.

As used herein, the term “complete remission” means that all signs and symptoms of cancer have disappeared

As used herein, the terms “recurrence” and “recurred,” as in a patient who “recurred,” means a cancer that is found after treatment, and after a period of time when the cancer couldn't be detected.

As used herein, the term “minimal residual disease” is used to describe a very small number of cancer cells that remain in the body during or after treatment.

As used herein, the terms “undetectable minimal residual disease” and “undetected minimal residual disease” means that no disease was detected after treatment using conventional imaging and laboratory tests, however, micrometastatic tumor cells still may or may not exist in the patient.

The terms “cancer progression” and “tumor progression” means the course of cancer becoming worse or spreading in the body, such as growth or increase in the size of a tumor or the appearance of new metastatic lesions.

The term “progression free survival” or “PFS” means the length of time during and after the treatment of a cancer, that a patient lives with the disease but it does not get worse and usually refers to situations in which a tumor is present as detected using by radiologic testing, laboratory testing, or clinically.

The term “event free survival” or “EFS” means the length of time after primary treatment for a cancer ends that the cancer patient remains free of certain complications or events that the treatment was intended to prevent or delay.

The term “early-stage patient” means a patient for which cancer is early in its growth and may not have spread to other parts of the body, or may not be detected or overt in other parts of the body, such as a patient with undetectable minimal residual disease.

The terms “patient in need thereof,” “patient in need,” “subject in need thereof,” or “subject in need” means a patient previously diagnosed with any cancer with a high risk for cancer recurrence or cancer progression as determined by one of a variety of different factors including, but not limited to, the type of cancer, the stage of detection, family history, genetic factors and biomarkers that a clinician may utilize for assessing such risk, such patient with or without prior treatment, with or without current treatment.

### B. METHODS OF IDENTIFYING A THERAPEUTIC AGENT

In one aspect, disclosed are methods of identifying a candidate therapeutic that has activity against a highly metastatic cell, the method comprising: (a) obtaining or having obtained highly metastatic cells; (b) culturing a first population of the highly metastatic cells in a 3D matrix under conditions sufficient to impose a metastatic endurance phenotype in the highly metastatic cells in the presence of a negative control, wherein the metastatic endurance phenotype is determined by measuring the total number of cells in the culture on at least three time points and determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under conditions that are substantially similar to those used to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; and (f) comparing the total number of cells from step b) and step c) on at least the third time point of step b), wherein when the total number of cells in the third time point of step c) is lower than the total number of cells in the third time point of step b), the candidate therapeutic has activity against a highly metastatic cell.

In one aspect, disclosed are methods of identifying a candidate therapeutic that has selective or specific activity against metastatic progression of a dormant cancer cell comprising: (a) obtaining or having obtained highly metastatic cells; (b) culturing a first population of the highly metastatic cells in a 3D matrix under conditions sufficient to impose a metastatic endurance phenotype in the highly metastatic cells in the presence of a negative control, wherein the metastatic endurance phenotype is determined by measuring the total number of cells in the culture on at least three time points and determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; (f) comparing the total number of cells from the third time point in step c) to the total number of cells from the third time point in step b), calculating the difference in the total number of cells as a ratio by [(third time point of step c−second time point of step b)/(third time point of step b−second time point of step b)]×100% or 100%−[(third time point of step c−second time point of step b)/(third time point of step b−second time point of step b)]×100; (g) identifying a candidate therapeutic capable of preventing or inhibiting proliferation, or capable of preventing, delaying, or reducing a switch or change to proliferation of a cell with a metastatic endurance phenotype, when the ratio of change in the total number of cells from step c) is less than the ratio of change in the total number of cells from step b) between the second and third time points; (h) culturing a fourth population of the highly metastatic cells from step a) in a 2D culture under conditions that promote proliferation and contacting the cultured proliferating highly metastatic cells with the candidate therapeutic from step c); (i) culturing a fifth population of the highly metastatic cells from step a) in a 2D culture under conditions that promote proliferation and contacting the cultured proliferating highly metastatic cells with the negative control from step b); (j) determining the total number of cells in the 2D cultures of step h) compared to step i) at the same time point and calculating a change in the number of cells as a ratio; and (k) comparing the ratio of step j) with the ratio of step f), wherein when the ratio is higher from step f) compared to step j) the candidate therapeutic has selective or specific activity against metastatic progression of a dormant cancer cell.

In one aspect, disclosed are methods of identifying a candidate therapeutic that has selective or specific activity against metastatic progression of a dormant cancer cell comprising: (a) obtaining or having obtained highly metastatic cells; (b) culturing a first population of the highly metastatic cells in a 3D matrix under conditions sufficient to impose a metastatic endurance phenotype in the highly metastatic cells in the presence of a negative control, wherein the metastatic endurance phenotype is determined by measuring the total number of cells in the culture on at least three time points and determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step c) is 75% or less the total number of cells from step b) indicates a metastatic endurance phenotype; (f) comparing the total number of cells from step b) and step c) on at least the third time point of step b) and calculating the % inhibition between the second and third time points by the equation: 100%−[(total # of cells at the third time point of step c)−(total # of cells at the second time point of step b)]/[(total # of cells the third time point of step b)−(total # of cells at the second time point of step b)]×100%; (g) culturing a fourth population of the highly metastatic cells from step a) in a 2D culture under conditions that promote proliferation and contacting the cultured proliferating highly metastatic cells with the candidate therapeutic from step c); (h) culturing a fifth population of the highly metastatic cells from step a) in a 2D culture under conditions that promote proliferation and contacting the cultured proliferating highly metastatic cells with the negative control from step b); (i) determining the total number of cells in the 2D cultures of step g) and h) at approximately the same time point and calculating % inhibition in the 2D cultures by the equation: 100%−[(the total number of cells from step g)/(the total number of cells from step h)×100%]; and (j) comparing the ratio of step i) with the ratio of step f), wherein a lower ratio of step f) compared to step i) identifies selective or specific activity in 3D vs 2D, indicating that the candidate therapeutic has selective or specific activity against metastatic progression of a dormant cancer cell.

In one aspect, disclosed are methods of identifying a candidate therapeutic, the method comprising: (a) obtaining or having obtained highly metastatic cells; (b) culturing a first population of the highly metastatic cells in a 3D matrix under conditions to impose a metastatic endurance phenotype in the highly metastatic cells in the presence of a negative control, wherein the metastatic endurance phenotype is determined by measuring the total number of cells in the culture on at least three time points, determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; and (f) comparing the total number of cells from step b) and step c) on at least the third time point of step b), wherein when the total number of cells in the third time point of step c) is lower than the total number of cells in the third time point of step b), the candidate therapeutic has activity against a highly metastatic cell.

In one aspect, disclosed are methods of identifying a candidate therapeutic that has selective or specific activity against metastatic progression of a microscopic metastatic tumor comprising: (a) obtaining or having obtained highly metastatic cells; (b) culturing a first population of the highly metastatic cells in a 3D matrix under conditions to impose a metastatic endurance phenotype in the highly metastatic cells in the presence of a negative control, wherein the metastatic endurance phenotype is determined by measuring the total number of cells in the culture on at least three time points, determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step d) is 75% or less the total number of cells from step b) indicates a metastatic endurance phenotype; (f) comparing the total number of cells from the third time point in step c) to the total number of cells from the third time point in step b), calculating the difference in the total number of cells as a ratio by [(third time point of step c−second time point of step b)/(third time point of step b−second time point of step b)]×100% or 100%−[(third time point of step c−second time point of step b)/(third time point of step b−second time point of step b)]×100; (g) identifying a candidate therapeutic capable of preventing or inhibiting proliferation, or capable of preventing, delaying or reducing a switch, or change to proliferation of a cell with a metastatic endurance phenotype, when the ratio of change in the total number of cells from step c) is less than the ratio of change in the total number of cells from step b) between the second and third time points; (h) culturing a fourth population of the highly metastatic cells from step a) in a 2D culture under conditions that promote proliferation and contacting the cultured proliferating highly metastatic cells with the candidate therapeutic from step c); (i) culturing a fifth population of the highly metastatic cells from step a) in a 2D culture under conditions that promote proliferation and contacting the cultured proliferating highly metastatic cells with the negative control from step b); (j) determining the total number of cells in the 2D cultures of step h) compared to step i) at the same time point and calculating a change in the number of cells as a ratio; and (k) comparing the ratio of step j) with the ratio of step f), wherein when the ratio is higher from step f) compared to step j) then the candidate therapeutic has selective or specific activity against metastatic progression of a microscopic metastatic tumor.

In one aspect, disclosed are methods of identifying a candidate therapeutic that has selective or specific activity against metastatic progression of a microscopic metastatic tumor comprising: (a) obtaining or having obtained highly metastatic cells; (b) culturing a first population of the highly metastatic cells in a 3D matrix under conditions to impose a metastatic endurance phenotype in the highly metastatic cells in the presence of a negative control, wherein the metastatic endurance phenotype is determined by measuring the total number of cells in the culture on at least three time points, determining the rate of growth between the first and second time point and the rate of growth between the second and third time point, and comparing the rate of growth between the first and second time points and the rate of growth between the second and third time points, wherein an increase of at least 10% in the rate of growth between the second and third time points compared to the rate of growth between the first and second time points indicates a metastatic endurance phenotype; (c) culturing a second population of the highly metastatic cells from step a) in a 3D matrix in the presence of a candidate therapeutic under the same conditions to impose a metastatic endurance phenotype in the highly metastatic cells as in step b) and measuring the total number of cells in culture on at least the third time point of step b); (d) optionally, culturing a third population of the highly metastatic cells from step a) in a 3D matrix in the presence of a positive control under the same conditions to impose a metastatic endurance phenotype in the highly metastatic cells as in step b) and measuring the total number of cells in the culture on at least the third time point of step b); (e) optionally, comparing the total number of cells from step b) and step d) on at least the third time point of step b); wherein when the total number of cells from step c) is 75% or less the total number of cells from step b) indicates a metastatic endurance phenotype; (f) comparing the total number of cells from step b) and step c) on at least the third time point of step b) and calculating the % inhibition between the second and third time points by the equation: 100%−[(total # of cells at the third time point of step c)−(total # of cells at the second time point of step b)]/[(total # of cells the third time point of step b)−(total # of cells at the second time point of step b)]×100%; (g) culturing a fourth population of the highly metastatic cells from step a) in a 2D culture under conditions that promote proliferation and contacting the cultured proliferating highly metastatic cells with the candidate therapeutic from step c); (h) culturing a fifth population of the highly metastatic cells from step a) in a 2D culture under conditions that promote proliferation and contacting the cultured proliferating highly metastatic cells with the negative control from step b); (i) determining the total number of cells in the 2D cultures of step g) and h) at the same time point and calculating % inhibition in the 2D cultures by the equation: 100%−[(the total number of cells from step g)/(the total number of cells from step h)×100%]; and (j) comparing the ratio of step i) with the ratio of step f), wherein a lower ratio of step f), compared to step i) identifies selective or specific activity in 3D vs 2D, indicating that the candidate therapeutic has selective or specific activity against metastatic progression of a microscopic metastatic tumor.

In one aspect, disclosed are methods of treating subjects in need of treatment for dormant cancer cells, micrometastatic cells, pre-recurrent cancer, or minimal residual disease (MRD), which refers to cancer cells remaining after treatment that cannot be detected by scans or tests. These cells have the potential to generate a larger mass or tumor, leading to eventual detection by scans and tests leading to relapse in patients. In a further aspect, disclosed are methods of preventing or treating recurrent cancer by treatment with particular therapeutic agents, as described herein and/or as identified by the disclosed methods.

In some aspects of the disclosed methods, the ratio of step i) with the ratio of step f) is calculated utilizing one or more selected from: (a) calculating the ratio of the IC₅₀ in 2D growth to that in 3D growth as calculated by:

$${IC}_{50} SI = \frac{{IC}_{50} ( 2 D )}{{IC}_{50} ( 3 D )} ;$$

(b) calculating the ratio of the 2D growth to that of 3D growth at concentrations producing an inhibition of growth, which would represent the ratio of minimum concentrations at which the agent would have detectable activity as calculated by:

$$MCAR = \frac{ICx ( 2 D )}{ICx ( 3 D )} ,$$

wherein each “x” is independently a % inhibition of growth; (c) calculating the % of growth in cell number in 3D [third time point vs. second time point] at highest 2D inactive dose concentration; (d) calculating the Lowest Active Dose Ratio Index (LADRI): I3D(C)/I2D(C) where I is the percent inhibition (defined as 10-25% inhibition in 2D) at concentration C and 2D and 3D represent 2D and 3D growth ratio respectively; and (e) calculating the Clinical Dose Selectivity Index (CDSI): I3D(C)/I2D(C) where I is the % inhibition at concentration C that is a selected dose in which 3D activity is adequate and 2D activity is low.

In some aspects of the disclosed methods, the negative control is media alone, media with a vehicle, media with vehicle and a compound known not to inhibit ME, or media with a compound known not to inhibit ME.

In some aspects of the disclosed methods, the positive control is media with vehicle and a compound known to inhibit ME or media with a compound known to inhibit ME.

In some aspects of the disclosed methods, the at least 10% in the rate of growth between the second and third time points compared to the rate of growth between the first and second time points of step b) is at least 10%, 20%, 30%, 40%, 50%, 100%, 150%, 200%, 250%, 300%, or higher than 300%.

In some aspects of the disclosed methods, step c) and step g) are repeated with different concentrations of the candidate therapeutic. In a further aspect, the different concentrations of the candidate therapeutic are tested and plotted on a graph.

In some aspects of the disclosed methods, step c) and step g) are repeated with several candidate therapeutics simultaneously.

In some aspects of the disclosed methods, the change in the total number of cells or the ratio of the changes in the total number of cells cultured with the different concentrations of the candidate therapeutic in the 3D matrix can be compared to the change in the total numbers of cells or to the ratio of the changes in the total number of cells cultured with different concentrations of the candidate therapeutic in 2D cultures.

In some aspects of the disclosed methods, a clinically relevant concentration of the candidate therapeutic is identified for selective activity against metastatic progression of a dormant cancer cell. In a further aspect, the clinically relevant concentration of the candidate therapeutic is identified when the candidate therapeutic reduces the ratio of the change in the total number of cells in the suitable 3D matrix while not reducing the ratio of change in the total number of cells in the 2D culture, or while reducing the ratio of change in the total number of cells in the 2D culture to a lesser extent, wherein the candidate therapeutic reduces the ratio of change in the total number of cells in step f) to a greater extent compared to the ratio of change in the total number of cells of step j).

In some aspects of the disclosed methods, comparing the ratio change in the total number of cells of step i) with the ratio change in the total number of cells from step f) comprises calculating a selectivity index (SI) of acting on the 3D matrix that induces the dormant or metastatic endurance phenotype over the proliferative phenotype that is expressed in 2D culture, wherein a candidate therapeutic has selective activity against a metastatic progression of a dormant cancer cell.

In some aspects of the disclosed methods, the highly metastatic cells are highly metastatic osteosarcoma cells.

In some aspects of the disclosed methods, one or more of steps b), c), and d) comprise culturing the highly metastatic cells in a 3D Basement Membrane Extract or SISgel.

In some aspects of the disclosed methods, during step b), an average increase of 25% per day or more in total number of cells or proliferation between the second and third time points as compared to between the first and second time points indicates a metastatic endurance phenotype.

In some aspects of the disclosed methods, during step b), the first time point is day 1, the second time point is day 3, the third time point is day 6, and 25% or more in average growth in cell number or proliferation between days 4-6 as compared to days 1-3 indicates a metastatic endurance phenotype.

In some aspects of the disclosed methods, during step b), the first time point is day 1, the second time point is day 3, the third time point is day 6, and 33% or more in average growth in cell number or proliferation between days 4-6 as compared to days 1-3 days indicates a metastatic endurance phenotype.

In some aspects of the disclosed methods, during step b), the first time point is between day 1 and 15, the second time point is 1 day after the first time point, and the third time point is 1 or more days after the second time point.

In some aspects of the disclosed methods, during step b), measuring cell numbers occurs between any of the three time points.

In some aspects of the disclosed methods, a candidate therapeutic known to have selective activity against a highly metastatic cell is used to determine that the metastatic endurance phenotype is expressed, and wherein the candidate therapeutic has selective activity against metastatic endurance.

In some aspects of the disclosed methods, the candidate therapeutic is rapamycin.

In some aspects of the disclosed methods, the candidate therapeutic identified is capable of preventing or inhibiting proliferation of a dormant cancer cell, preventing or inhibiting the transitioning of a dormant cancer cell to a rapidly proliferating cancer cell, or killing a dormant cancer cell.

In some aspects of the disclosed methods, the candidate therapeutic is a small molecule compound, a biologic, or a cell.

In some aspects of the disclosed methods, step c) and step g) are repeated with different concentrations of the candidate therapeutic. In a further aspect, the change in the total number of cells cultured with the different concentrations of the candidate therapeutic in the 3D matrix is compared to the total numbers of cells cultured with different concentrations of the candidate therapeutic in 2D cultures.

In some aspects of the disclosed methods, a clinically relevant concentration of the candidate therapeutic is identified for selective activity against metastatic progression of a dormant cancer cell when the candidate therapeutic reduces the change in the total number of cells in the suitable 3D matrix while not reducing the change in the total number of cells in the 2D culture.

In some aspects of the disclosed methods, the ratio of step i) with the ratio of step f) is calculated utilizing one or more selected from: (a) calculating the ratio of the IC₅₀ in 2D growth to that in 3D growth as calculated by:

$${IC}_{50} SI = \frac{{IC}_{50} ( 2 D )}{{IC}_{50} ( 3 D )} ;$$

(b) calculating the ratio of the 2D growth to that of 3D growth at concentrations producing an inhibition of growth, which would represent the ratio of minimum concentrations at which the agent would have detectable activity as calculated by:

$$MCAR = \frac{ICx ( 2 D )}{ICx ( 3 D )} ;$$

wherein each “x” is independently a % inhibition of growth; (c) calculating the % of growth in cell number in 3D [third time point vs. second time point] at highest 2D inactive dose concentration; (d) calculating the Lowest Active Dose Ratio Index (LADRI): I3D(C)/I2D(C) where I is the percent inhibition (defined as 10-25% inhibition in 2D) at concentration C and 2D and 3D represent 2D and 3D growth ratio respectively; and (e) calculating the Clinical Dose Selectivity Index (CDSI): I3D(C)/I2D(C) where I is the % inhibition at concentration C that is a selected dose in which 3D activity is adequate and 2D activity is low.

In some aspects of the disclosed methods, the negative control is media alone, media with a vehicle, media with vehicle and a compound known not to inhibit ME, or media with a compound known not to inhibit ME. In some aspects of the disclosed methods, the positive control is media with vehicle and a compound known to inhibit ME or media with a compound known to inhibit ME.

In some aspects of the disclosed methods, the at least 10% in the rate of growth between the second and third time points compared to the rate of growth between the first and second time points of step b) is at least 10%, 20%, 30%, 40%, 50%, 100%, 150%, 200%, 250%, 300%, or higher than 300%.

In some aspects of the disclosed methods, step c) and step g) are repeated with different concentrations of the candidate therapeutic. In some aspects of the disclosed methods, the different concentrations of the candidate therapeutic are tested and plotted on a graph.

In some aspects of the disclosed methods, step c) and step g) are repeated with several candidate therapeutics simultaneously.

In some aspects of the disclosed methods, the change in the total number of cells or the ratio of the changes in the total number of cells cultured with the different concentrations of the candidate therapeutic in the 3D matrix can be compared to the change in the total numbers of cells, or the ratio of the changes in the total number of cells cultured with different concentrations of the candidate therapeutic in 2D cultures.

In some aspects of the disclosed methods, a clinically relevant concentration of the candidate therapeutic is identified for selective activity against metastatic progression of a microscopic metastatic tumor. In some aspects of the disclosed methods, the clinically relevant concentration of the candidate therapeutic is identified when the candidate therapeutic reduces the ratio of the change in the total number of cells in the suitable 3D matrix while not reducing the ratio of change in the total number of cells in the 2D culture, or reducing the ratio of change in the total number of cells in the 2D culture to a lesser extent, wherein the candidate therapeutic reduces the ratio of change in the total number of cells in step f) to a greater extent compared to the ratio of change in the total number of cells of step j).

In some aspects of the disclosed methods, comparing the ratio change in the total number of cells of step i) with the ratio change in the total number of cells from step f) comprises calculating a selectivity index (SI) of acting on the 3D matrix that induces the metastatic endurance phenotype over the proliferative phenotype that is expressed in 2D culture, wherein a candidate therapeutic has selective activity against a metastatic progression of a microscopic metastatic tumor.

In some aspects of the disclosed methods, a combination of candidate therapeutic is tested for selective activity against metastatic progression of a microscopic metastatic tumor, wherein two or more drugs are tested in combination, wherein the effect of a combination is compared to the effect of each drug used in the combination alone, and wherein the combination effect determined using the Highest Single Agent approach.

In some aspects of the disclosed methods, one or more of steps b), c), and d) comprise culturing the highly metastatic cells in a 3D Basement Membrane Extract or SISgel.

In some aspects of the disclosed methods, during step b), an average increase of 25% per day or more in total number of cells or proliferation between the second and third time points as compared to between the first and second time points indicates a metastatic endurance phenotype.

In some aspects of the disclosed methods, during step b), the first time point is day 1, the second time point is day 3, the third time point is day 6, and 25% or more in average growth in cell number or proliferation between days 4-6 as compared to days 1-3 indicates a metastatic endurance phenotype.

In some aspects of the disclosed methods, during step b), the first time point is day 1, the second time point is day 3, the third time point is day 6, and 33% or more in average growth in cell number or proliferation between days 4-6 as compared to days 1-3 indicates a metastatic endurance phenotype.

In some aspects of the disclosed methods, during step b), the first time point is between day 1 and 15, the second time point is 1 day after the first time point, and the third time point is 1 or more days after the second time point.

In some aspects of the disclosed methods, during step b), measuring cell numbers occurs between any of the three time points.

In some aspects of the disclosed methods, a candidate therapeutic known to have selective activity against a highly metastatic cell is used to determine that the metastatic endurance phenotype is expressed, and wherein the candidate therapeutic has selective activity against metastatic endurance.

In some aspects of the disclosed methods, the candidate therapeutic is rapamycin.

In some aspects of the disclosed methods, the candidate therapeutic identified is capable of preventing or inhibiting proliferation of a microscopic metastatic tumor, preventing or inhibiting the transitioning of a microscopic metastatic tumor to a rapidly proliferating cancer cell, or killing a microscopic metastatic tumor cancer cell.

In some aspects of the disclosed methods, the candidate therapeutic is a small molecule compound, a biologic, or a cell.

In some aspects of the disclosed methods, step c) and step g) can be repeated with different concentrations of the candidate therapeutic or combination. In some aspects of the disclosed methods, the change in the total number of cells cultured with the different concentrations of the candidate therapeutic or combination in the 3D matrix can be compared to the total numbers of cells cultured with different concentrations of the candidate therapeutic or combination in 2D cultures.

In some aspects of the disclosed methods, a clinically relevant concentration of the candidate therapeutic or combination is identified for selective activity against metastatic progression of a microscopic metastatic tumor when the candidate therapeutic or combination reduces the change in the total number of cells in the suitable 3D matrix while not reducing the change in the total number of cells in the 2D culture.

In one aspect, disclosed are methods of treating subjects in need of treatment for microscopic metastatic tumors. These cells or tumors have the potential to generate a larger mass or tumor, leading to eventual detection by scans and tests leading to relapse in patients. In a further aspect, disclosed are methods of preventing or treating recurrent cancer by treatment with particular therapeutic agents, as described herein and/or as identified by the disclosed methods.

In another aspect, disclosed are methods for targeting of Metastatic Endurance of DTCs, and disabling or weakening it, and by doing so preventing, delaying it, or reducing the severity of cancer recurrence. Disclosed are methods for screening and identifying drugs that target ME, for selecting dosing regimen of such drugs and drug combinations and enabling their utility as therapeutics for prevention of cancer recurrence.

In another aspect, disclosed are methods for targeting of Metastatic Endurance of microscopic metastatic tumor, and disabling or weakening it, and by doing so preventing, delaying it, or reducing the severity of cancer recurrence. Disclosed are methods for screening and identifying drugs that target ME, for selecting dosing regimen of such drugs and drug combinations and enabling their utility as therapeutics for prevention of cancer recurrence.

In one aspect, disclosed are methods for selecting dosing regimen appropriate for various types of drugs and drug combinations and enable their utility as therapeutics for prevention of cancer recurrence in osteosarcoma and other cancers.

Disclosed herein are methods of analysis that are based on culturing the highly metastatic cells in a 3D matrix under conditions to impose a metastatic endurance phenotype in the highly metastatic cells. In some aspects, the conditions can include different 3D matrices, reduced growth-factor matrices, single cell suspension in 3D matrix, manipulation of culture components such as exogenous extracellular matrix proteins (e.g., fibronectin, collagen), growth factors and/or nutrients.

Disclosed herein are methods that comprise, in part, culturing the highly metastatic cells in a 3D matrix under conditions to impose a metastatic endurance phenotype in the highly metastatic cells and subsequently, culturing the cells in a 2D culture under conditions that promote proliferation.

Disclosed herein are methods of identifying a candidate therapeutic that has selective activity against metastatic progression of a dormant cancer cell comprising: (a) obtaining or having obtained highly metastatic cells; (b) culturing the highly metastatic cells in a 3D matrix under conditions to impose a metastatic endurance phenotype in the highly metastatic cells, wherein the metastatic endurance phenotype is determined by comparing the change in the total number of cells in the culture daily for at least six days and comparing the change in the total number of cells from a first period of days and a second period of days, wherein an increase in proliferation between the second period of days as compared to the first period of days indicates a metastatic endurance phenotype; (c) culturing a first portion of the highly metastatic cells in a 3D matrix under conditions to impose a metastatic endurance phenotype in the highly metastatic cells and contacting the first portion of highly metastatic cells with a candidate therapeutic; (d) culturing a second portion of the highly metastatic cells in a 3D matrix under conditions to impose a metastatic endurance phenotype in the highly metastatic cells and contacting the second portion of highly metastatic cells with a control; (e) determining the change in the change in the total number of cells from step (c) and step (d) over a time course; (f) comparing the change in the change in the total number of cells from step (c) to the change in the total number of cells of a step (d) over the time course; (g) identifying a candidate therapeutic capable of preventing or inhibiting proliferation of a cell with a metastatic endurance phenotype, when the change in the total number of cells from step (c) is the same or less than the change in the total number of cells from step (d) after six days of culture; (h) culturing the highly metastatic cells from step (b) in a 2D culture under conditions that promote proliferation, contacting the cultured proliferating highly metastatic cells with a candidate therapeutic such as small molecule compound, a biologic or a cell such as an NK cell or other immune cell, or any other biologically active substance; (i) determining the change in the change in the total number of cells in the 2D culture of step (h); (j) comparing the change in the change in the total number of cells of step (i) with the change in the change in the total number of cells from step (c); wherein when the candidate therapeutic reduces the change in the total number of cells in step (c) to a greater extent compared to the change in the total number of cells of step (i), the candidate therapeutic has selective activity against a metastatic progression of a dormant cancer cell. In some aspects, the time course of step (f) is between 0 and 10 days. In some aspects, the time course of step (f) is 0-6 days. In some aspects, the change in the change in the total number of cells from step (c) to the change in the total number of cells of a step (d) calculated in step (f) is six days.

In some aspects of the disclosed methods, step c) and step h) can be repeated with different concentrations of the candidate therapeutic.

In some aspects of the disclosed methods, the change in the total number of cells cultured with the different concentrations of the candidate therapeutic in the 3D matrix can be compared to the total numbers of cells cultured with different concentrations of the candidate therapeutic in 2D cultures.

In some aspects of the disclosed methods, a clinically relevant concentration of the candidate therapeutic can be identified for selective activity against metastatic progression of a dormant cancer cell when the candidate therapeutic reduces the change in the total number of cells in the suitable 3D matrix while not reducing the change in the total number of cells in the 2D culture.

In some aspects of the disclosed methods, comparing the change in the total number of cells of step i) with the change in the total number of cells from step e) comprises calculating a selectivity index (SI) of acting on the 3D matrix that induces the dormant or ME phenotype over the proliferative phenotype that is expressed in 2D culture. In some aspects, the selectivity can be determined by:
calculating the ratio of the IC₅₀ in 2D growth to that in 3D growth as calculated by:

$${IC}_{50} SI = \frac{{IC}_{50} ( 2 D )}{{IC}_{50} ( 3 D )} ;$$

calculating the ratio of the 2D growth to that of 3D growth at concentrations producing an inhibition of growth, which would represent the ratio of minimum concentrations at which the agent would have detectable activity as calculated by:

$$MCAR = \frac{ICx ( 2 D )}{ICx ( 3 D )} ;$$

wherein each “x” is independently a % inhibition of growth;
calculating the % growth in cell number in 3D [third time point vs. second time point] at highest 2D inactive dose concentration;
calculating the Lowest Active Dose Ratio Index (LADRI): % inhibition in 3D [Day 6 vs. Day 3] at dose C divided by % inhibition in 2D at dose C (wherein the dose C should be selected by a dose that leads to 10-25% inhibition in 2D); or
calculating the Clinical Dose Selectivity Index (CDSI): I3D(C)/I2D(C) where I is the % inhibition at concentration C that is a selected dose in which 3D activity is adequate and 2D activity is low.

In some aspects of the disclosed methods, the highly metastatic cells are highly metastatic osteosarcoma cells.

In some aspects, steps (b), (c), or (d) comprises culturing the highly metastatic cells in a 3D Basement Membrane Extract or a SISgel.

In some aspects of the disclosed methods, during step (b), an increase of 25% or more in proliferation or cell number in a period of 2 or more days as compared to an initial period of at least one day and up to 10 days indicates a metastatic endurance phenotype. In some aspects of the disclosed methods, during step (b), an increase of 33% or more in proliferation or cell number between days 4-6 as compared to days 1-3 days indicates a metastatic endurance phenotype.

In some aspects of the disclosed methods, the control in step (d) comprises a therapeutic known to reduce the total number of highly metastatic cells, a vehicle, or a composition that does not comprise a therapeutic.

In some aspects of the disclosed methods, the first period of days is 1-3 days and the second period of days is 2-4 days and in some aspects of the disclosed methods, the first period of days is 3 days (days 1 to 3) and the second period of days is 3 days (days 4 to 6).

In some aspects of the disclosed methods, the candidate therapeutic identified in step (g) is capable of preventing or inhibiting proliferation of a dormant cancer cell, preventing or inhibiting the transitioning of a dormant cancer cell to a rapidly proliferating cancer cell, or killing a dormant cancer cell.

In one aspect, disclosed are methods for selecting a dosing regimen appropriate for various types of drugs and drug combinations and enabling their utility as a therapeutic for prevention of cancer recurrence in osteosarcoma and other cancers.

Disclosed herein are methods of analysis that are based on culturing the highly metastatic cells in a 3D matrix under conditions to impose a metastatic endurance phenotype in the highly metastatic cells. In some aspects, the conditions can include different 3D matrices, reduced growth-factor matrices, single cell suspension in 3D matrix, manipulation of culture components such as exogenous extracellular matrix proteins (e.g., fibronectin, collagen), growth factors and/or nutrients.

Disclosed herein are methods that comprise, in part, culturing the highly metastatic cells in a 3D matrix under conditions to impose a metastatic endurance phenotype in the highly metastatic cells and subsequently, culturing the cells in a 2D culture under conditions that promote proliferation.

Disclosed herein are methods of identifying a candidate therapeutic that has selective activity against metastatic progression of a microscopic metastatic tumor comprising: (a) obtaining or having obtained highly metastatic cells; (b) culturing the highly metastatic cells in a 3D matrix under conditions to impose a metastatic endurance phenotype in the highly metastatic cells, wherein the metastatic endurance phenotype is determined by comparing the change in the total number of cells in the culture daily for at least six days and comparing the change in the total number of cells from a first period of days and a second period of days, wherein an increase in proliferation between the second period of days as compared to the first period of days indicates a metastatic endurance phenotype; (c) culturing a first portion of the highly metastatic cells in a 3D matrix under conditions to impose a metastatic endurance phenotype in the highly metastatic cells and contacting the first portion of highly metastatic cells with a candidate therapeutic; (d) culturing a second portion of the highly metastatic cells in a 3D matrix under conditions to impose a metastatic endurance phenotype in the highly metastatic cells and contacting the second portion of highly metastatic cells with a control; (e) determining the change in the change in the total number of cells from step (c) and step (d) over a time course; (f) comparing the change in the change in the total number of cells from step (c) to the change in the total number of cells of a step (d) over the time course; (g) identifying a candidate therapeutic capable of preventing or inhibiting proliferation of a cell with a metastatic endurance phenotype, when the change in the total number of cells from step (c) is the same or less than the change in the total number of cells from step (d) after six days of culture; (h) culturing the highly metastatic cells from step (b) in a 2D culture under conditions that promote proliferation, contacting the cultured proliferating highly metastatic cells with a candidate therapeutic such as small molecule compound, a biologic or a cell such as an NK cell or other immune cell, or any other biologically active substance; (i) determining the change in the change in the total number of cells in the 2D culture of step (h); (j) comparing the change in the change in the total number of cells of step (i) with the change in the change in the total number of cells from step (c); wherein when the candidate therapeutic reduces the change in the total number of cells in step (c) to a greater extent compared to the change in the total number of cells of step (i), the candidate therapeutic has selective activity against a metastatic progression of a microscopic metastatic tumor. In some aspects, the time course of step (f) is between 0 and 10 days. In some aspects, the time course of step (f) is 0-6 days. In some aspects, the change in the change in the total number of cells from step (c) to the change in the total number of cells of a step (d) calculated in step (f) is six days.

In some aspects of the disclosed methods, step c) and step h) can be repeated with different concentrations of the candidate therapeutic.

In some aspects of the disclosed methods, the change in the total number of cells cultured with the different concentrations of the candidate therapeutic in the 3D matrix can be compared to the total numbers of cells cultured with different concentrations of the candidate therapeutic in 2D cultures.

In some aspects of the disclosed methods, a clinically relevant concentration of the candidate therapeutic can be identified for selective activity against metastatic progression of a microscopic metastatic tumor when the candidate therapeutic reduces the change in the total number of cells in the suitable 3D matrix while not reducing the change in the total number of cells in the 2D culture.

In some aspects of the disclosed methods, comparing the change in the total number of cells of step i) with the change in the total number of cells from step e) comprises calculating a selectivity index (SI) of acting on the 3D matrix that induces the microscopic metastatic tumor or ME phenotype over the proliferative phenotype that is expressed in 2D culture. In some aspects, the selectivity can be determined by:
calculating the ratio of the IC50 in 2D growth to that in 3D growth as calculated by:

$$IC 50 SI = \frac{IC 50 ( 2 D )}{IC 50 ( 3 D )} ;$$

calculating the ratio of the 2D growth to that of 3D growth at concentrations producing an inhibition of growth, which would represent the ratio of minimum concentrations at which the agent would have detectable activity as calculated by:

$$MCAR = \frac{ICx ( 2 D )}{ICx ( 3 D )} ;$$

wherein each “x” is independently a % inhibition of growth;
calculating the % growth in cell number in 3D [third time point vs. second time point] at highest 2D inactive dose concentration;
calculating the Lowest Active Dose Ratio Index (LADRI): % inhibition in 3D [Day 6 vs. Day 3] at dose C divided by % inhibition in 2D at dose C (wherein the dose C should be selected by a dose that leads to 10-25% inhibition in 2D); or
calculating the Clinical Dose Selectivity Index (CDSI): I3D(C)/I2D(C) where I is the % inhibition at concentration C that is a selected dose in which 3D activity is adequate and 2D activity is low.

In some aspects of the disclosed methods, the highly metastatic cells are highly metastatic osteosarcoma cells.

In some aspects, steps (b), (c), or (d) comprises culturing the highly metastatic cells in a 3D Basement Membrane Extract or a SISgel.

In some aspects of the disclosed methods, during step (b), an increase of 25% or more in proliferation or cell number in a period of 2 or more days as compared to an initial period of at least one day and up to 10 days indicates a metastatic endurance phenotype. In some aspects of the disclosed methods, during step (b), an increase of 33% or more in proliferation or cell number between days 4-6 as compared to days 1-3 days indicates a metastatic endurance phenotype.

In some aspects of the disclosed methods, the control in step (d) comprises a therapeutic known to reduce the total number of highly metastatic cells, a vehicle, or a composition that does not comprise a therapeutic.

In some aspects of the disclosed methods, the first period of days is 1-3 days and the second period of days is 2-4 days and in some aspects of the disclosed methods, the first period of days is 3 days (days 1 to 3) and the second period of days is 3 days (days 4 to 6).

In some aspects of the disclosed methods, the candidate therapeutic identified in step (g) is capable of preventing or inhibiting proliferation of a dormant cancer cell, preventing or inhibiting the transitioning of a dormant cancer cell to a rapidly proliferating cancer cell, or killing a dormant cancer cell.

In some aspects of the disclosed methods, the candidate therapeutic identified according to above is capable of preventing or inhibiting proliferation of a microscopic metastatic tumor, preventing or inhibiting the transitioning of a microscopic metastatic tumor to a rapidly proliferating or growing cancer cell or tumor, or killing a microscopic metastatic tumor.

In some aspects the disclosed methods can be multiplexed and run in parallel with multiple candidate therapeutics or the same candidate therapeutics at different concentrations.

In some aspects of the disclosed methods, anticancer drugs can be identified that will prevent, or control, or cure micrometastatic disease and/or MRD, and or dormant cancer cells which is the true killer of many cancer patients can be tested using any of the above in vitro and/or ex vivo, BME, SISgel, PuMA or similar methods. Previous studies have shown that when cancer cells are grown on an extracellular matrix (ECM) preparation derived from normal (non-malignant) tissue (“normal ECM”), the malignant phenotype of the cells is suppressed, and they radically change their appearance and growth characteristics to appear more normal. Cancer cells growing on this normal matrix are representative of suppressed micrometastatic cancer cells (cancer cells having a suppressed malignant phenotype). Finding therapeutic agents that are more active against cancer cells cultured on a normal matrix than they are against cells growing on 2D, plastic (or other “permissive” media) can identify therapeutic agents active against dormant cancer cells and phenotypically dormant and/or suppressed micrometastases. In addition, finding therapeutic agents that can either kill, or inhibit cancer cells cultured on various types of matrix such as BME or SISgel will identify therapeutic agents active against dormant cancer cells.

In some aspects of the disclosed methods, anticancer drugs can be identified that will prevent, or control, or cure microscopic metastatic tumor, micrometastatic disease and/or MRD, which is the true killer of many cancer patients can be tested using any of the above in vitro and/or ex vivo, BME, SISgel, PuMA, or similar methods. Previous studies have shown that when cancer cells are grown on an extracellular matrix (ECM) preparation derived from normal (non-malignant) tissue (“normal ECM”), the malignant phenotype of the cells is suppressed, and they radically change their appearance and growth characteristics to appear more normal. Cancer cells growing on this normal matrix are representative of suppressed micrometastatic cancer cells (cancer cells having a suppressed malignant phenotype). Finding therapeutic agents that are more active against cancer cells cultured on a normal matrix than they are against cells growing on 2D, plastic (or other “permissive” media) can identify therapeutic agents active against microscopic metastatic tumors. In addition, finding therapeutic agents that can either kill, or inhibit cancer cells cultured on various types of matrix such as BME or SISgel will identify therapeutic agents active against microscopic metastatic tumors.

In some aspects, finding therapeutic agents that can keep cancer cells cultured on various types of matrix such as polyacrylamide (PAM) in certain conditions can identify therapeutic agents active against dormant cancer cells. In some aspects, finding therapeutic agents that can keep cancer cells cultured on various types of matrix such as polyacrylamide (PAM) in certain conditions can identify therapeutic agents active against microscopic metastatic tumor.

In some aspects, finding therapeutic agents that can keep cancer cells cultured ex vivo, such as using PuMA in certain conditions can identify therapeutic agents active against dormant cancer cells. In some aspects, finding therapeutic agents that can keep cancer cells cultured ex vivo, such as using PuMA in certain conditions can identify therapeutic agents active against microscopic metastatic tumor.

According to some aspects, novel and/or existing therapeutic agents are effective against dormant cancer cells that are representative of cells of pre-recurrent or recurrent tumors. According to some aspects, novel and/or existing therapeutic agents are effective against microscopic metastatic tumors that are representative of cells of pre-recurrent or recurrent tumors.

According to some aspects, novel and/or existing therapeutic agents are effective against dormant cancer cells and prevent, delay, and/or attenuate cancer recurrence at distant site. According to some aspects, novel and/or existing therapeutic agents are effective against microscopic metastatic tumors and prevent, delay, and/or attenuate cancer recurrence at distant site.

According to some aspects, novel and/or existing therapeutic agents are effective against dormant cancer cells and can prevent, delay, and/or attenuate cancer recurrence at the local site of a primary tumor. According to some aspects, novel and/or existing therapeutic agents are effective against microscopic metastatic tumors and can prevent, delay, and/or attenuate cancer recurrence at local site of a primary tumor.

According to some aspects, therapeutic agents that target dormant cancer cells are used in a more optimal and suitable manner for cancer patients. In one aspect, methods of use and/or a dosing regimen are more suitable for development and for clinical trials and as products for cancer patients compared to therapeutic agents or compound uses that have been previously tested in vitro and/or in vivo. The therapeutic agents or their uses have been designed to improve one or more features such as potency, bioavailability, duration, safety, and reasonable pharmaceutical properties, as well as selectivity, efficacy, and dose-proportionality.

According to some aspects, therapeutic agents that target microscopic metastatic tumors are used in a more optimal and suitable manner for cancer patients. In one aspect, methods of use and/or a dosing regimen are more suitable for development and for clinical trials and as products for cancer patients compared to therapeutic agents or compound uses that have been previously tested in vitro and/or in vivo. The therapeutic agents or their uses have been designed to improve one or more features such as potency, bioavailability, duration, safety, and reasonable pharmaceutical properties, as well as selectivity, efficacy and dose-proportionality.

In some aspects, therapeutic agents that can target dormant cancer cells are formulated to make them more suitable for development and for clinical trials and as products for cancer patients. Such formulation can include encapsulation, small release formulation, means for improving the potency, bioavailability, duration, safety, and/or reasonable pharmaceutical properties of a potential drug, as well as selectivity, efficacy and dose-proportionality.

In some aspects, therapeutic agents that can target microscopic metastatic tumors are formulated to make them more suitable for development and for clinical trials and as products for cancer patients. Such formulation can include encapsulation, small release formulation, means for improving the potency, bioavailability, duration, safety, and reasonable pharmaceutical properties of a potential drug, as well as selectivity, efficacy and dose-proportionality.

In some aspects, therapeutic agents that may target dormant cancer cells are administered in a dosing regimen formulated to make them more suitable for development and for clinical trials and as products for cancer patients and improve selectivity, safety, efficacy and/or dose-proportionality and/or enable long term administration, and/or combination with other drugs, procedures and standard of care. In some aspects, therapeutic agents that may target microscopic metastatic tumors are administered in a dosing regimen formulated to make them more suitable for development and for clinical trials and as products for cancer patients and improve selectivity, safety, efficacy and/or dose-proportionality and/or enable long term administration, and/or combination with other drugs, procedures and standard of care.

In some aspects, the target oncology product profile for disabling dormant cancer cells before they can cause recurrence is a low-dose, long term safe drug to prevent cancer recurrence and death with an adequate safety and tolerability profile. In some aspects, the target oncology product profile for disabling microscopic metastatic tumors before they can cause recurrence is a low-dose, long term safe drug to prevent cancer recurrence and death with an adequate safety and tolerability profile.

According to some aspects, therapeutic agents that may target dormant cancer cells are combined with each other or with other drugs and treatments for cancer. As examples, such therapeutic agents can be added to standard of care, different drug regimen, radiation and or surgery and/or ablation and they can be given to patients in an adjuvant or a neo adjuvant setting, or any time post initial treatment or primary or metastatic tumor detection.

According to some aspects, therapeutic agents that may target microscopic metastatic tumors are combined with each other or with other drugs and treatments for cancer. As examples, such therapeutic agents can be added to standard of care, different drug regimen, radiation and or surgery and/or ablation and they can be given to patients in an adjuvant or a neo adjuvant setting, or any time post initial treatment or primary or metastatic tumor detection.

According to some aspects, therapeutic agents that may target dormant cancer cells are combined with each other or with other drugs and treatments for cancer. As an example, such therapeutic agents can be added to standard of care, they can be given to patients after metastatic cancer has been detected in order to prevent, delay, and/or attenuate additional metastases.

According to some aspects, therapeutic agents that may target microscopic metastatic tumors are combined with each other or with other drugs and treatments for cancer. As an example, such therapeutic agents can be added to standard of care, they can be given to patients after metastatic cancer has been detected in order to prevent, delay, and/or attenuate additional metastases.

According to some aspects, therapeutic agents that target metastatic endurance of dormant cancer cells, include the therapeutic agents listed below, act in certain cancer types on dormant cancer cells that have been disseminated to secondary sites and prevent their transition out of dormancy to form deadly recurrences, or kill the dormant cancer cells via apoptosis, or other mechanisms. Such therapeutic agents may also act indirectly on various components of the tumor microenvironment. They may also act on various components of the body, including the immune system.

According to some aspects, therapeutic agents that target metastatic endurance of microscopic metastatic tumors, include the therapeutic agents listed below, act in certain cancer types on microscopic metastatic tumors that have been disseminated to secondary sites and prevent their transition out of dormancy, no growth or slow growth to form deadly recurrences, or kill the microscopic metastatic tumors via apoptosis, or other mechanisms. Such therapeutic agents may also act indirectly on various components of the tumor microenvironment. They may also act on various components of the body, including the immune system.

Many targets are or have been viewed as attractive targets for cancer therapy, yet so far drugs and drugs classes that are believed to target them either as direct or indirect inhibitors or as agonists have not been proven efficacious in oncology. According to one aspect, the lack of sufficient efficacy may result from the nature of these drugs, which necessitates a different treatment paradigm compared to previous settings in which drug was given in a stage of disease progression in which the cancer was to advance and where there was limited duration of treatment.

Based on evidence in cancer models, treatment with such therapeutic agents is mostly likely to be effective in cancer patients if. 1) applied in patients with early cancer stages without evidence of metastasis but at risk for recurrence, as determined by factors including but not limited to the type of cancer, stage of detection, biomarkers, age of the patient, and available diagnostic tests, and, 2) administered for a long term or chronically, (therefore, once the drug treatment is stopped the effect of the drug on inhibiting the dormant cancer cells from existing dormancy to form deadly metastases is diminished or non-existent). Specific patient populations most likely to benefit most from such treatment include early-stage patients with undetectable minimal residual disease, meaning patients with various cancer where despite conventional imaging and laboratory tests relevant to the cancer type no minimal residual disease was detectable.

As detailed herein, a patient with early cancer stages without evidence of metastasis but at risk for recurrence can be identified by a variety of different factors including, but not limited to, the type of cancer, the stage of detection, biomarkers, the age of the patient, and the available diagnostic tests. Examples of cancers with high risks for deadly recurrences, such as, for example, a risk of at least 20%, at least 30%, at least 40%, at least 50%, at least 60%, at least 70%, at least 80%, at least 90%, at least 95%, or at least 99% chance of recurrence, which can benefit by treatments of the invention herein include, but are not limited to, osteosarcoma, breast cancer, renal cancer, melanoma, and colorectal cancer. Osteosarcoma has a recurrence rate of 30-40% for patients with primary disease and further recurrences in about 80% or more of patients following an initial recurrence. In breast cancer, standard diagnostic testing can identify if the cancer is a triple negative breast cancer, which has a higher metastatic recurrence rate than other types of breast cancer, of about 25% within about three years of successful treatment of localized disease with a 5-year survival rate of about 77%. The recurrence rate is highest in the first 3 years after treatment but falls off at the 5-year mark. In renal cancer, after nephrectomy, the incidence of renal cell carcinoma recurrence has been reported to be 7% with a median time of 38 months for T1 tumors, 26% with a median time of 32 months for T2 disease, and 39% with a median time to recurrence at 17 months for T3 tumors. In melanoma, at least 8% of stage IA+B and 30% of Stage IIA+B recur. More than 6% of all patients recur 10 or more years after initial diagnosis. In colorectal cancer, the rate of recurrence in stage 1 patients is ˜25% after 5 years.

Based on evidence in cancer models, treatment with such therapeutic agents is likely to be most effective in cancer patients if. 1) applied in patients with early cancer stages without evidence of metastasis but at risk for recurrence, as determined by factors including but not limited to the type of cancer, stage of detection, biomarkers, age of the patient, and available diagnostic tests, and, 2) administered for a sufficient duration of time, which may be long term but not necessarily chronically. Specific patient populations most likely to benefit most from such treatment include early-stage patients with minimal residual disease that may be undetectable by most standard diagnostic procedures, meaning patients with various cancer where despite conventional imaging and laboratory tests relevant to the cancer type no minimal residual disease was detectable.

Based on evidence in cancer models, treatment with such therapeutic agents may also be effective in cancer patients if. 1) applied in patients that have metastatic recurrence that has been detected and treated such that patients are in partial or complete remission, and, 2) administered for a sufficient duration of time, which may be long term or chronically. Specific patient populations most likely to benefit most from such treatment include patients who recurred and are in remission.

Disclosed herein are new paradigms of long-term daily treatment beginning in the adjuvant and/or neoadjuvant treatment stages of early-stage patients with the cancers as detailed herein is required in order to achieve efficacy in cancer. The clinical utility using certain drugs requires chronic or long-term daily treatment beginning as early as possible after initial cancer diagnosis. Such treatments can begin in the adjuvant and/or neoadjuvant treatment stages of early-stage patients with various cancers. In such treatment, the drugs will be given for as long as possible and ideally for the entire life span of the patient.

Disclosed herein are new paradigms of long-term daily treatment beginning in the adjuvant and/or neoadjuvant treatment stages of patients who have had recurrences that have been treated such that the patients are again in partial or complete remission as detailed herein is required in order to achieve efficacy in cancer. The clinical utility using certain drugs requires chronic or long-term daily treatment beginning as early as possible after diagnosis of recurrent cancer. Such treatments can begin in the adjuvant and/or neoadjuvant treatment stages of such patients with various cancers. In such treatment, the drugs may need to be given for as long as possible but not necessarily chronically.

In some aspects of the methods disclosed herein, various drugs can be used in combination with other therapeutic agents. The combinations can include combinations of drugs that are in different classes, targeting different pathways or networks, or drugs that target different parts of the same pathway or network. Combinations can also include combinations of drugs for which the targets are not known, but the drugs are determined empirically in cell cultures or in animals or in humans to provide benefit.

Importantly, data generated by various companies, or institutions or clinicians in cancer patients often supports the long-term safety and tolerability of such drugs, and therefore their utility as a long-term treatments for preventing cancer recurrence in early-stage cancer patients who have a risk for recurrence.

### C. THERAPEUTIC AGENTS

Disclosed herein are treatment methods comprising administering a compound identified by the disclosed screening method to a subject in need thereof. In various aspects, the compound is an active pharmaceutical ingredient (API) selected from one or more of: a mTOR inhibitor; a heat shock protein 90 (HSP90) inhibitor; a heat shock protein 70 (HSP70) inhibitor; an unfolded protein response (UPR) inhibitor; a modulator of nicotinamide adenine dinucleotide (NAD+) metabolism; a glutamyl-prolyl tRNA synthetase inhibitor; a glutaminase inhibitor; a poly(ADP)-ribose polymerase (PARP) inhibitor; a DNA-damaging agent; an agent capable of generating reactive oxygen species (ROS); a survivin inhibitor; a MEK 1 and/or a MEK 2 inhibitor; a Bcl-xL and/or Bcl-2 inhibitor; a proteosome inhibitor; a muscarinic acetylcholine (mACh) antagonist; a B-Raf inhibitor; an AXL kinase inhibitor; a NEDD8 inhibitor; an aurora kinase inhibitor; a histone deacetylase (HDAC) inhibitor (e.g., a Class I selective histone deacetylase (HDAC) inhibitor); a bromodomain (BET) inhibitor; a PI3K inhibitor; an Akt inhibitor; a LSD inhibitor; a DNA-PK inhibitor; an IGF1R inhibitors; and/or a PLK inhibitor.

Thus, in various aspects of the invention, the disclosed treatment method comprises administering a mTOR inhibitor. The possible role of mTOR inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the mTOR inhibitor is rapamycin. In other preferred embodiments of the invention, the mTOR inhibitor includes, but is not limited to, a rapalog (or derivative of rapamycin). In further aspects of the invention, the rapamycin rapalog includes, but is not limited to, ridaforolimus, temsirolimus, everolimus, and tacrolimus. In further embodiments of the invention, the mTOR inhibitor includes, but is not limited to, a non-rapalog. In further embodiments, the non-rapalog includes, but is not limited to, AZD8055, Vistusertib (AZD2014), and Sapanisertib. In other embodiments of the invention, the mTOR inhibitor includes, but is not limited to, ICSN3250, LY3023414, OSU-53, gedatolisib/PF05212384, PI-103, BEZ235, voxtalisib/XL765, PP242, or OSI-027.

Thus, in various aspects of the invention, the disclosed treatment method comprises administering a Hsp90 inhibitor. The possible role of Hsp90 inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the Hsp90 inhibitor is alvespimycin. In other embodiments of the invention the Hsp90 inhibitor includes, but is not limited to BIIB021, tanespimycin or retaspimycin. In further aspects of the invention, the Hsp90 inhibitor includes, but is not limited to, 17-AAG, ganetispib, IPI-504, luminespib, PEN-866, XL888, AYU922, onalespib/AT13387, or Debio0932.

Thus, in various aspects of the invention, the disclosed treatment method comprises administering a modulator of NAD metabolism. The possible role of modulating NAD metabolism in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the modulator of NAD metabolism is darporinad. In other preferred embodiments of the invention the modulator of NAD metabolism includes, but is not limited to β-Lapachone and its analogues. In further aspects of the invention, the modulator of NAD metabolism includes, but is not limited to, APO866, CHS-828/GMX1778, GMX1777, KPT-9274/ATG-019, OT-82, apaziquone, 17-DMAG, streptonigrin, tanshinone IIA, dunnione, ARQ501, or ARQ761.

Thus, in various aspects of the invention, the disclosed treatment method comprises administering a glutamyl-prolyl tRNA synthetase inhibitor. The possible role of inhibiting glutamyl-prolyl tRNA synthetase in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the glutamyl-prolyl tRNA synthetase inhibitor is halofuginone.

Thus, in various aspects of the invention, the disclosed treatment method comprises administering a glutaminase inhibitor. The possible role of glutaminase inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the glutaminase inhibitor is telaglenastat.

Thus, in various aspects of the invention, the disclosed treatment method comprises administering a PARP inhibitor. The possible role of PARP inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the PARP inhibitor is talazoparib. In other preferred embodiments of the invention the PARP inhibitor includes, but is not limited to, olaparib, rucaparib, niraparib, veliparib, or fluzoparib.

Thus, in various aspects of the invention, the disclosed treatment method comprises administering a Hsp70 inhibitor. The possible role of Hsp70 inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the Hsp70 inhibitor is minnelide or triptolide. In further aspects of the invention, the Hsp70 inhibitor includes, but is not limited to, VER-155008, NSC630668-R/1, MAL3-101, DMT3132, DMT3024, MAL2-111B, 15-deoxyspergualin, quercetin, kahweol, cantharidin, apoptozole, MKT-077, YM-1, JG-83, JG-84, or JG-98.

Thus, in various aspects of the invention, the disclosed treatment method comprises administering an unfolded protein response (UPR) inhibitor. The possible role of UPR inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the UPR inhibitor is minnelide or triptolide. In further aspects of the invention, the UPR inhibitor includes, but is not limited to, MKC-3946, 4μ8C, STF-083010, KIRA6, B-109, GSK2606414, GSK2656157, AMG PERK44, Melatonin, Ceapins, IRSIB, or AID2732.

Thus, in various aspects of the invention, the disclosed treatment method comprises administering a DNA damaging (and ROS inducing) agent. The possible role of DNA damaging (and ROS inducing) agents in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In the most preferred embodiment of the invention, the DNA damaging (and ROS inducing) agent is mitomycin C. In further aspects of the invention, the DNA damaging (and ROS inducing) agent includes, but is not limited to, melphalan, bendamustine, cisplatin, carboplatin, oxaliplatin, gemcitibine, Ara-C, etoposide, doxorubicin, C-1027, bleomycin, GSK2795039, CPP11G, CPP11H, thioridazine, Ewha-18278/APX-115, setanaxib/GKT137831, L-NMMA, 2-iminobiotin, VAS203, allopurinol, febuxostat, topiroxostat, safinamide, selegiline, rasagiline, toloxatone, pirlindole, phenelzine, isocarboxazid, tranlycypromine, moclobemide, AZD3241, AZD4831, vitamin C, vitamin E, tigecyclin, doxycycline, clarithromycin, niclosamide, hydroxychloroquine, simvastatin, digoxin, or fluphenazine.

Thus, in various aspects of the invention, the disclosed treatment method comprises administering a survivin inhibitor. The possible role of survivin inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the survivin inhibitor is YM-155. In further aspects of the invention, the survivin inhibitor includes, but is not limited to, AICAR, UC-112, withanone, piperine, PZ-6-QN, Abbott 8, LLP3, LLP9, S12, indinavir, LQZ-7, LQZ-7F, FL 118, irinotecan, SN-38, topotecan, camptothecin, SF002-96-1, or WM-127.

Thus, in various aspects of the invention, the disclosed treatment method comprises administering a MEK inhibitor. The possible role of MEK inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In further aspects of the invention, the MEK inhibitor inhibits MEK1 or MEK2. In further aspects of the invention, the MEK inhibitor inhibits MEK1 and MEK2. In the most preferred embodiment of the invention, the MEK inhibitor is PD032590. In other aspects of the invention the MEK inhibitor includes, but is not limited to pimasertib or binimetinib. In further aspects of the invention, the MEK inhibitor includes, but is not limited to, trametinib, cobimetinib, CI-1040/PD184352, PD0325901, selumetinib/AZD6244, MEK162, AZD8330, TAK-733, GDC-0623, refametinib, R04987655/CH4987655, RO5126766, WX-554, or HL-085.

Thus, in various aspects of the invention, the disclosed treatment method comprises administering a Bcl-xL/Bcl-2 inhibitor. The possible role of Bcl-xL/Bcl-2 inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the Bcl-xL/Bcl-2 inhibitor is navitoclax. In further aspects of the invention, the Bcl-xL/Bcl-2 inhibitor includes, but is not limited to, ABT-737, ABT-263, ABT-199, venetoclax, APG-1252, AZD0466, A-1331852, or ABBV-155.

Thus, in various aspects of the invention, the disclosed treatment method comprises administering a proteosome inhibitor. The possible role of proteosome inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the proteosome inhibitor is oprozomib. In further aspects of the invention, the proteosome inhibitor includes, but is not limited to, bortezomib, carfilzomib, ixazomib, delanzomib, marizomib, or capzimin.

Thus, in various aspects of the invention, the disclosed treatment method comprises administering a Raf inhibitor. The possible role of Raf inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the Raf inhibitor is dabrafenib. In further aspects of the invention, the Raf inhibitor includes, but is not limited to, vemurafenib, trametinib, encorafenib, or binimetinib.

Thus, in various aspects of the invention, the disclosed treatment method comprises administering a AXL inhibitor. The possible role of AXL inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the AXL inhibitor is bemcentinib. In further aspects of the invention, the AXL inhibitor includes, but is not limited to, TP-0903, crizotinib/PF-02341066/Xalkori, bosutinib/SKI-606/Bosulif, gilteritinib/ASP2215, S49076, amuvatinib/MP-470, sunitinib/Sutent, SNS3142-D08, UNC2025, SGI-7079, UNC569, NA80x1, or DP-3975.

Thus, in various aspects of the invention, the disclosed treatment method comprises administering a NEDD8-activating enzyme (NAE) inhibitor. The possible role of NEDD8-activating enzyme (NAE) inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In a preferred embodiment of the invention, the NEDD8-activating enzyme (NAE) inhibitor is pevonedistat. In further aspects of the invention, the NEDD8-activating enzyme (NAE) inhibitor includes, but is not limited to, TAS4464, ZM223, DI-591, or NAcM-OPT.

Thus, in various aspects of the invention, the disclosed treatment method comprises administering an aurora A kinase inhibitor. The possible role of aurora A kinase inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In the most preferred embodiment of the invention, the aurora A kinase inhibitor is alisertib or dansuertib. In further aspects of the invention, the aurora A kinase inhibitor includes, but is not limited to, AMG900, AS703569, AT9283, BI-847325, CYC116, ENMD-2076, tozasertib/MK-0457, MK-5108/VX-689, MLN8054, PF-03814735, danusertib/PHA-739358, SNS-314, CYC3, AKI603, BPR1K0609S1, LDD970, MK-8745, LY3295668, BPR1K653, TY-011, BPR1K871, SCH-1473759, derrone, JNJ-7706621, SAR156497, R1498, VE-465, CCT129202, CCT137690, PHA-680632, AKI-001, or reversine.

Thus, in various aspects of the invention, the disclosed treatment method comprises administering a HDAC inhibitor. The possible role of HDAC inhibitors in targeting, inhibiting, or preventing metastatic endurance of dormant cancer cells, microscopic metastatic tumors or minimal residual disease (which can be detectable or non-detectable), is summarized in Table 1 under “MOA Rationale for ME.” In further aspects, the HDAC inhibitor is a Class I HDAC inhibitor. In a preferred embodiment of the invention, the HDAC inhibitor is domatinostat. In further aspects of the invention, the HDAC inhibitor includes, but is not limited to, vorinostat/SATA, belinostat/PXD-101, panobinostat/LBH-589, trichostatine A, quisiniostat/JNJ-16241199, WW437, pivaloyloxmethyl butyrate/AN-9, sodium butyrate, sodium phenylbutyrate, valproate, entinostat/MS-275, tacedinaline/Cp-994, mocetinostat/MG-0103, cambinol, EX-527, sirtinol, nicotinamide, ABHA, CBHA, I-7ab, RGFP966, PC134051, C149, ricolinostat/ACY-1215, tubacine, depudecin, SEN196, COMPOUND 6J, or JGB1741.

In a further aspect, classes and specific examples of therapeutic agents useful with the disclosed treatment methods include, but are not limited to, those listed in Table 1 and Table 2.

TABLE 1

| Drug Class | Therapeutic agents | MOA Rationale for ME |
|---|---|---|
| mTOR inhibitor-Rapalogs | Rapamycin and all rapalogs such | The mTOR network is known |
| | as Ridaforolimus, Temsirolimus, | to regulate gene transcription |
| | Everolimus, Tacrolimus | and protein synthesis to regulate |
| | | cell proliferation and also plays |
| | | an important role in tumor |
| | | metabolism. In ME, cells are |
| | | undergoing metabolic changes |
| | | throughout, as well as |
| | | maintaining protein synthesis |
| | | during dormancy and outbreak |
| | | in ME. Inhibiting this pathway |
| | | can potentially prevent |
| | | proliferation during outbreak as |
| | | well as inhibit metabolism |
| | | necessary to keep cells alive |
| | | during ME |
| mTOR inhibitor-Non- | AZD8055, Vistusertib | The mTOR network is known |
| Rapalogs | (AZD2014), Sapanisertib | to regulate gene transcription |
| | | and protein synthesis to regulate |
| | | cell proliferation and also plays |
| | | an important role in tumor |
| | | metabolism. In ME, cells are |
| | | undergoing metabolic changes |
| | | throughout, as well as |
| | | maintaining protein synthesis |
| | | during dormancy and outbreak |
| | | in ME. Inhibiting this pathway |
| | | can potentially prevent |
| | | proliferation during outbreak as |
| | | well as inhibit metabolism |
| | | necessary to keep cells alive |
| | | during ME |
| HSP90 inhibitor | Alvespimycin, BIIB021, | Hsp90 is a cell stress response |
| | Tanespimycin, Retaspimycin | protein that has diverse effects |
| | | on cell cycle, survival, hormone |
| | | signaling, protein degradation, |
| | | apoptosis and cell homeostasis. |
| | | ME most likely exerts cellular |
| | | stress on tumor cells and Hsp90 |
| | | is heavily implicated in the cell |
| | | stress response, allowing cells, |
| | | particularly tumor cells, to |
| | | survive inhospitable conditions |
| | | such as those felt by cells |
| | | undergoing ME |
| Modulator of NAD | Daporinad, B-lapachone and its | The NAD metabolic pathway is |
| Metabolism | analogues | implicated not only in energy |
| | | metabolism, but DNA damage |
| | | and stress response. Cells |
| | | undergoing ME have unique |
| | | metabolic requirements and |
| | | often upregulate stress response |
| | | pathways, thus targeting this |
| | | network may disrupt multiple |
| | | pathways critical to ME. |
| Glutamyl-prolyl tRNA | Halofuginone | Known to interact with Hsp90, |
| synthetase | | it is important in RNA |
| | | translation and inhibition of this |
| | | protein may shut down stress- |
| | | mediated (potentially IRES) |
| | | protein synthesis that may be |
| | | critical for the upregulation of |
| | | ME programs that keep cells |
| | | alive during therapeutic stress |
| | | and stress from the TME. |
| Glutaminase inhibitor | Telaglenastat | Glutaminase is critical in the |
| | | glutaminolysis pathway that |
| | | directly affects cell metabolism. |
| | | GLS1 is also implicated as a |
| | | promoter of cancer-specific |
| | | proliferation. Due to its effects |
| | | on proliferation and |
| | | metabolism, glutaminase may |
| | | have an important role in ME |
| | | by regulating metabolism and |
| | | proliferation both during |
| | | dormancy and outbreak. |
| PARP | Talazoparib | PARP is critical in DNA |
| | | damage response (keeping cells |
| | | alive despite rapid division |
| | | causing DNA damage), and |
| | | plays a role in transcription and |
| | | chromatin remodeling. ME is |
| | | characterized by the ability to |
| | | survive traditional |
| | | chemotherapy that often relies |
| | | on DNA damage as its ultimate |
| | | MOA. Inhibiting this network |
| | | may prevent ME from keeping |
| | | cell alive during ME and block |
| | | transcription and epigenetic |
| | | alteration that is also implicated |
| | | in ME-related processes. |
| HSP70 | Minnelide, Triptolide | Hsp70 is a chaperone protein |
| | | that can control hormone |
| | | receptors, kinases (Raf, eIF2a, |
| | | CyclinB1) and transcription |
| | | factors (c-Myc, pRb). Many of |
| | | these pathways are implicated |
| | | in cancer, and in particular, in |
| | | ME there is most likely a |
| | | reliance on IRES-mediated |
| | | translation during stress to keep |
| | | the cells alive. Altering this |
| | | pathway may affect the ability |
| | | of cells undergoing ME to |
| | | continue to survive. |
| Unfolded Protein Response | Minnelide, triptolide | ER stress plays a significant |
| (UPR) | | role in tumor cells. As for |
| | | cancer development, UPR can |
| | | induce tumor cell survival, |
| | | progression, and metastasis |
| | | under unfavorable conditions, |
| | | such as hypemic hypoxia, |
| | | accumulation of reactive |
| | | oxygen species, and ATP as |
| | | well as nutrient deprivation. |
| | | Inhibiting this pathway may |
| | | prevent the ME-related effects |
| | | from the UPR. |
| DNA damage (and ROS- | Mitomycin C | While this is classically thought |
| inducing) | | of as targeting rapidly-dividing |
| | | cells, DNA damaging agents |
| | | may also induce apoptosis by |
| | | promoting further DNA damage |
| | | that passes a tipping point |
| | | where ME cells that are |
| | | generally tolerant to DNA |
| | | damage die. This may be |
| | | important during dormancy |
| | | (cells survive despite earlier |
| | | DNA damage) or outbreak |
| | | (period of rapid cell division |
| | | typically characterized by |
| | | higher rates of DNA damage). |
| Survivin | YM-155 | Survivin is an important anti- |
| | | apoptotic protein that is often |
| | | upregulated in cancer. By |
| | | targeting this protein, we may |
| | | be able to overcome the ability |
| | | of cells to survive therapeutic |
| | | intervention and the |
| | | inhospitable TME. |
| | | Upregulation can also promote |
| | | tumor proliferation which is |
| | | also important to block during |
| | | ME. |
| MEK | Pimasertib, PD0325901, | MEK/ERK plays a crucial role |
| | Binimetinib | in cell proliferation and |
| | | apoptosis, particularly in |
| | | cancer. By targeting this |
| | | pathway, these drugs may be |
| | | able to overcome the ME |
| | | programs that lead to |
| | | uncontrolled cell growth during |
| | | outbreak, causing apoptosis in |
| | | ME cells that would otherwise |
| | | survive other insults (chemo, |
| | | TME, etc.) |
| BCL-XL/BCL-2 | Navitoclax | Bcl-xL/Bcl-2 is an important |
| | | anti-apoptotic protein that is |
| | | often upregulated in cancer. By |
| | | targeting this protein, we may |
| | | be able to overcome the ability |
| | | of cells to survive therapeutic |
| | | intervention and the |
| | | inhospitable TME. |
| | | Upregulation can also promote |
| | | tumor proliferation which is |
| | | also important to block during |
| | | ME. |
| Proteosome | Oprozomib | The proteosome helps maintain |
| | | protein homeostasis in the cell |
| | | through coordinated protein |
| | | degradation. Protein levels and |
| | | misfolding are regulated by the |
| | | proteosome. In cancer, the |
| | | proteosome can be hijacked to |
| | | degrade proteins that can |
| | | suppress tumor formation (e.g. |
| | | p53, c-Myc, c-jun, c-Fos), or to |
| | | prevent degradation of |
| | | oncogenes (e.g. Raf, Myc, Rel, |
| | | Src) to promote tumorigenesis. |
| | | Alterations in proteosome |
| | | function also alter the |
| | | expression of inflammatory |
| | | cytokines, altering the immune |
| | | environment. Alterations in the |
| | | proteosome are one potential |
| | | way that tumors can upregulate |
| | | ME-related programs without |
| | | genetic mutations. |
| antimuscarinic | Tropicamide | |
| RAF | Dabrafenib | Raf plays a crucial role in cell |
| | | proliferation as a node in the |
| | | Ras/Raf/MEK/Erk network and |
| | | is frequently mutated in various |
| | | cancers, leading to aberrant |
| | | overactivity. By targeting this |
| | | pathway, these drugs may be |
| | | able to inhibit the ME programs |
| | | that lead to uncontrolled cell |
| | | growth during outbreak. |
| Bromodomain (BET) | JQ1, mivebresib, AZD5153 | BETs are also important in |
| | | epigenetic modification of cells |
| | | (similar to HDACs), and if the |
| | | early dissemination of tumor |
| | | cells is correct, epigenetic |
| | | alterations are likely the main |
| | | way that cells undergo ME |
| | | (timeframe is too short to be |
| | | genetic mutations). Blocking |
| | | this pathway may prevent cells |
| | | from initiating ME programs. |
| AXL | Bemcentinib | The AXL/GAS6 network is |
| | | responsible for a variety of |
| | | functions that support the ME |
| | | phenotype, including |
| | | proliferation, invasion, drug |
| | | resistance and enhanced |
| | | metastasis. By targeting this |
| | | pathway, a variety of ME- |
| | | related activities can be |
| | | inhibited (see, e.g., Zhu et al. |
| | | (2019) Mol Cancer). |
| NEDD8-activating enzyme | Pevonedistat | Neddylation is a |
| (NAE) inhibitor | | posttranslational modification |
| | | that adds Nedd8 (similar to |
| | | ubiquitin) to proteins, |
| | | controlling their function. |
| | | Nedd8 activating enzyme E1 |
| | | (very similar to E3 ligases) |
| | | initiates this cascade by loading |
| | | activated Nedd8 to itself. In |
| | | cancer, overactivation of this |
| | | network leads to degradation of |
| | | tumor suppressors like p21 & |
| | | p27, leading to cancer |
| | | progression. This may have an |
| | | impact on ME by removing |
| | | tumor suppressors that would |
| | | keep cell dormant or lead to |
| | | apoptosis. Blocking this |
| | | network can lead to cell cycle |
| | | arrest, apoptosis, senescence |
| | | and autophagy due to build up |
| | | of neddylated proteins. |
| Aurora A kinase | Alisertib, Danusertib | Aurora kinases are critical for |
| | | cell division, and aurora kinase |
| | | A favors the G2/M transition |
| | | promoting mitosis. In cancers, |
| | | AurA is a tumor promoter that |
| | | promotes cell proliferation, and |
| | | survival. Cells undergoing ME |
| | | require survival in the face of |
| | | inhospitable conditions and |
| | | need to initiate proliferation |
| | | during outbreak, and AurA may |
| | | be implicated in this. |
| HDAC | Domatinostat | HDACs are important in |
| | | epigenetic modification of cells, |
| | | and if the early dissemination of |
| | | tumor cells is correct, |
| | | epigenetic alterations are likely |
| | | the main way that cells undergo |
| | | ME (timeframe is too short to |
| | | be genetic mutations). Blocking |
| | | this pathway may prevent cells |
| | | from initiating ME programs. |

TABLE 2

| Class | Description | Examples |
|---|---|---|
| mTOR | Therapeutic agents that can disrupt | ICSN3250, LY3023414, OSU-53, |
| | the formation of the MTORC1/2 | gedatolisib/PF05212384, PI-103, |
| | complexes or inhibits its enzymatic | BEZ235, voxtalisib/XL765, PP242, |
| | activity | OSI-027, Tacrolimus |
| Hsp90 | Therapeutic agents that inhibit the | 17-AAG, ganetispib, IPI-504, |
| | function (directly or through | BIIB021, luminespib, PEN-866, |
| | allosteric binding) of heat shock | XL888, AYU922, |
| | protein 90 | onalespib/AT13387, Debio0932 |
| UPR | Therapeutic agents that disrupt the | MKC-3946, 4μ8C, STF-083010, |
| | cell signaling response to ER stress | KIRA6, B-109, GSK2606414, |
| | from misfolded proteins (IRE1, | GSK2656157, AMG PERK44, |
| | PERK, ATF6, eIF2α & CHOP are | Melatonin, Ceapins, IRSIB, |
| | major components of this signaling | AID2732 |
| | network) | |
| Hsp70 | Therapeutic agents that inhibit the | VER-155008, NSC630668-R/1, |
| | function (directly or through | MAL3-101, DMT3132, DMT3024, |
| | allosteric binding) of heat shock | MAL2-11B, 15-deoxyspergualin, |
| | protein 70 | quercetin, kahweol, cantharidin, |
| | | apoptozole, MKT-077, YM-1, JG- |
| | | 83, JG-84, JG-98 |
| NAD Modulators | Therapeutic agents that disrupt the | APO866, CHS-828/GMX1778, |
| | levels or function of nicotinamide | GMX1777, KPT-9274/ATG-019, |
| | adenine dinucleotide (key metabolic | OT-82, apaziquone, 17-DMAG, |
| | and signaling molecule; NQO1 and | streptonigrin, tanshinone IIA, |
| | NAMPT are major components of | dunnione, ARQ501, ARQ761 |
| | this signaling network) | |
| glutamyl-prolyl tRNA | Therapeutic agents that inhibit the | — |
| synthetase | function (directly or through | |
| | allosteric binding) of glutamyl-prolyl | |
| | tRNA synthase, which is critical to | |
| | protein translation | |
| Glutaminase | Therapeutic agents that inhibit the | BPTES, telaglenastat/CB-839, |
| | function (directly or through | Compound 968, DON, JHU-083, |
| | allosteric binding) of glutaminase | acivicin, azaserine, GPNA, V-9302 |
| | protein, a key modulator of | |
| | glutamine metabolism | |
| PARP | Therapeutic agents that inhibit the | olaparib, rucaparib, niraparib, |
| | function (directly or through | talazoparib, veliparib, fluzoparib |
| | allosteric binding) of PARP proteins, | |
| | which are critical in the DNA | |
| | damage stress response | |
| DNA Damaging | Therapeutic agents that induce | Melphalan, bendamustine, cisplatin, |
| | sufficient DNA damage to lead to | carboplatin, oxaliplatin, gemcitibine, |
| | cancer cell death preferentially | Ara-C, etoposide, doxorubicin, C- |
| | | 1027, bleomycin |
| ROS Generating | Therapeutic agents that generate | GSK2795039, CPP11G, CPP11H, |
| | sufficient reactive oxygen species to | thioridazine, Ewha-18278/APX-115, |
| | lead to cancer cell death | setanaxib/GKT137831, L-NMMA, |
| | preferentially | 2-iminobiotin, VAS203, allopurinol, |
| | | febuxostat, topiroxostat, safinamide, |
| | | selegiline, rasagiline, toloxatone, |
| | | pirlindole, phenelzine, |
| | | isocarboxazid, tranlycypromine, |
| | | moclobemide, AZD3241, AZD4831, |
| | | vitamin C, vitamin E, tigecyclin, |
| | | doxycycline, clarithromycin, |
| | | niclosamide, hydroxychloroquine, |
| | | simvastatin, digoxin, fluphenazine |
| Survivin | Therapeutic agents that inhibit the | AICAR, UC-112, withanone, |
| | function (directly or through | piperine, PZ-6-QN, Abbott 8, LLP3, |
| | allosteric binding) of survivin | LLP9, S12, indinavir, LQZ-7, LQZ- |
| | protein | 7F, FL118, irinotecan, SN-38, |
| | | topotecan, camptothecin, SF002-96- |
| | | 1, WM-127 |
| MEK1/2 | Therapeutic agents that inhibit the | Trametinib, cobimetinib, CI- |
| | function (directly or through | 1040/PD184352, PD0325901, |
| | allosteric binding) of MEK1/2 | selumetinib/AZD6244, MEK162, |
| | protein | AZD8330, TAK-733, GDC-0623, |
| | | refametinib, |
| | | RO4987655/CH4987655, |
| | | RO5126766, WX-554, HL-085 |
| Bcl-xL/Bcl-2 | Therapeutic agents that inhibit the | ABT-737, ABT-263, ABT-199, |
| | function (directly or through | venetoclax, APG-1252, AZD0466, |
| | allosteric binding) of Bcl-xL/Bcl-2 | A-1331852, ABBV-155 |
| | protein | |
| Proteosome | Therapeutic agents that inhibit the | Bortezomib, carfilzomib, ixazomib, |
| | function or stability of protein | delanzomib, marizomib, capzimin |
| | complexes that are critical to | |
| | maintaining protein homeostasis | |
| | through proteolysis/degradation | |
| mACh | Therapeutic agents that inhibit the | VU319, MK-7622, TAK-071, |
| | function (directly or through | HTL0018318, HTL0016878 |
| | allosteric binding) of mACh | |
| | signaling | |
| B-Raf | Therapeutic agents that inhibit the | Vemurafenib, trametinib, |
| | function (directly or through | encorafenib, binimetinib |
| | allosteric binding) of B-Raf protein | |
| BET | Therapeutic agents that inhibit the | AZD5153, ABBV-075, BMS- |
| | function (directly or through | 986158, CPI-0610, GSK525762, |
| | allosteric binding) of BET protein | OTX-015, INCB054329, |
| | | INCB057643, FT-1101, CC-90010, |
| | | ODM-207, PLX51107, JQ1, I- |
| | | BET762, I-BET151, RVX-208, |
| | | MS417, ABBV-744, SJ432 |
| AXL | Therapeutic agents that inhibit the | Bemcentinib/R428/BGB324, TP- |
| | function (directly or through | 0903, crizotinib/PF- |
| | allosteric binding) of AXL protein | 02341066/Xalkori, bosutinib/SKI- |
| | | 606/Bosulif, gilteritinib/ASP2215, |
| | | S49076, amuvatinib/MP-470, |
| | | sunitinib/Sutent, SNS3142-D08, |
| | | UNC2025, SGI-7079, UNC569, |
| | | NA80x1, DP-3975 |
| NEDD8 | Therapeutic agents that inhibit the | Pevonedistat/MLN4924, TAS4464, |
| | function (directly or through | ZM223, DI-591, NAcM-OPT, |
| | allosteric binding) of NEDD8 | |
| | protein | |
| Aurora kinase | Therapeutic agents that inhibit the | AMG900, AS703569, AT9283, BI- |
| | function (directly or through | 847325, CYC116, ENMD-2076, |
| | allosteric binding) of Aurora kinase | tozasertib/MK-0457, MK-5108/VX- |
| | protein | 689, MLN8054, PF-03814735, |
| | | danusertib/PHA-739358, SNS-314, |
| | | CYC3, AKI603, BPR1K0609S1, |
| | | LDD970, MK-8745, LY3295668, |
| | | BPRIK653, TY-011, BPRIK871, |
| | | SCH-1473759, derrone, JNJ- |
| | | 7706621, SAR156497, R1498, VE- |
| | | 465, CCT129202, CCT137690, |
| | | PHA-680632, AKI-001, reversine |
| HDAC | Therapeutic agents that inhibit the | Vorinostat/SAHA, belinostat/PXD- |
| | function of HDAC proteins that are | 101, panobinostat/LBH-589, |
| | responsible for chromatin | trichostatine A, quisiniostat/JNJ- |
| | remodeling and epigenetic regulation | 16241199, WW437, |
| | | pivaloyloxmethyl butyrate/AN-9, |
| | | sodium butyrate, sodium |
| | | phenylbutyrate, valproate, |
| | | entinostat/MS-275, tacedinaline/CI- |
| | | 994, mocetinostat/MG-0103, |
| | | cambinol, EX-527, sirtinol, |
| | | nicotinamide, ABHA, CBHA, I-7ab, |
| | | RGFP966, PCI34051, C149, |
| | | ricolinostat/ACY-1215, tubacine, |
| | | depudecin, SEN196, COMPOUND |
| | | 6J, JGB1741 |
| IGF1R | Therapeutic agents that inhibit the | Picropodophyllin (AXL1717), |
| | function (directly or through | Linsitinib, BMS-754807, BVP |
| | allosteric binding) of IGF1R protein | 51004, XL228, INSM-18 (NDGA |

### D. THERAPEUTIC COMBINATIONS

Disclosed herein are treatment methods comprising administering a compound identified by the disclosed screening method to a subject in need thereof. In various aspects, a combination therapy for targeting microscopic metastatic tumor including two or more drugs may be more efficacious and may enable achieving reduced toxicity and drug exposure requirements, inhibition the emergence of acquired therapeutic resistance to any single drug and enhanced activity. A combination therapy may include separate pills, fixed dose combinations include but are not limited to the following: when given to a patient orally as separate pills, as a fixed dose pill, or parentally, or in combination of an oral, or parenteral administration, one or more times a day, less than once a day, together or in sequence. Due to toxicity and tolerability concerns, it may also be advantageous to optimize efficacy and safety/tolerability by using a drug combination is manner in which two drugs are used in combination for a certain time period and thereafter only one of the two drugs is administered for an extended period of time.

In many cancers, and specifically in osteosarcoma, epigenetic dysregulation results in alterations in the expression of a variety of genes involved in cancer, particularly involving the dysregulation of signaling networks and pathways related to metastasis. Metastasis-specific enhancer regions are regulated via the epigenome and are mediators of the expression of genes critical to promoting metastasis. The epigenome can be modified through the use of epigenomic modulators and regulators, including but limited to histone deacetylase (HDAC), lysine-specific demethylase (LSD) and bromodomain and extraterminal (BET) protein inhibitors. Inhibiting the activity of epigenome modulators or regulators is a promising approach to targeting cancer cells.

Histone deacetylase inhibitors (HDACs) regulate gene transcription. They regulate the epigenome via deacetylation of histones on chromatin and result in control of the expression of a variety of genes involved in cancer. HDACs inhibitors might be reverse the activation of tumor suppressor genes. They also directly deacetylate certain proteins (such as P53, Myc). HDACs are divided into several classes that have different tissue and sub cellular level distributions and functions: Class I—HDAC 1, 2, 3 & 8, Class IIa—HDAC 4, 5, 7 & 9, Class IIb—HDAC 6 & 10, Class III, also known as the sirtuins (SIRTs), include SIRT1-7 and Class IV—HDAC 11. HDACs are frequently overactive in cancers, leading to aberrant proliferation, cell cycle dysfunction, increased angiogenesis and resistance to apoptosis. In the bonce cancer osteosarcoma (OS), HDAC inhibitors, including domatinostat and others have shown in vitro and in vivo efficacy in OS models. Class I—HDAC proteins are upregulated in many cancers and have been found to be critical for tumor cell proliferation, and are implicated in the increased expression of oncoproteins and the down regulation of tumor suppressor genes. For these reasons, specific targeting of Class I HDACs may be an attractive approach to targeting cancer cells.

The mTOR network is known to regulate diverse activities, including: gene transcription, protein synthesis, lipid biosynthesis, tumor cell metabolism, apoptosis, proteosome assembly. In ME, cells rely on the above to become less sensitive to apoptosis, and enable them to adapt to stress, survive and enter at some point an enhanced growth state. Inhibiting mTOR to disable these activities causes cytostatic (or DormaStatic) effects that prevent the ability to survive and outbreak to enhanced growth.

Cooperativity, mTOR+HDAC led to significant decrease in oncogenic Myc protein level, to decreased Pro-tumorigenic TBX2 & E2F1 transcription factor activity, to upregulated P53, CDKN2A & RB1 tumor suppressors and MHC-II upregulation. Since the mTOR and HDAC networks regulate a broad range of proteins and the epigenome, which regulate critical cancer cell functions such as enhanced gene transcription, protein synthesis, metabolism, proteome assembly and apoptosis, which are essential for stress adaptation, survival and the ability to support transition to as state of rapid proliferation, mTOR and HDAC inhibition can act both independently (in parallel) and cooperatively. Most of HDAC and mTOR networks are non-overlapping and therefore provide significant opportunity to prevent acquired resistance to any one critical node in the network.

Thus, in various aspects of the invention, the disclosed treatment methods comprise administering two or more active pharmaceutical ingredients (APIs) selected from a mTOR inhibitor; a heat shock protein 90 (HSP90) inhibitor; a heat shock protein 70 (HSP70) inhibitor; an unfolded protein response (UPR) inhibitor; a modulator of nicotinamide adenine dinucleotide (NAD+) metabolism; a glutamyl-prolyl tRNA synthetase inhibitor; a glutaminase inhibitor; a poly(ADP)-ribose polymerase (PARP) inhibitor; a DNA-damaging agent; an agent capable of generating reactive oxygen species (ROS); a survivin inhibitor; a MEK 1 and/or MEK 2 inhibitor; a Bcl-xL and/or Bcl-2 inhibitor; a proteosome inhibitor; a muscarinic acetylcholine (mACh) antagonist; a B-Raf inhibitor; an AXL kinase inhibitor; a NEDD8 inhibitor; an aurora kinase inhibitor; a histone deacetylase (HDAC) inhibitor (e.g., a Class I selective histone deacetylase (HDAC) inhibitor); a bromodomain (BET) inhibitor; a PI3K inhibitor; an Akt inhibitor; a LSD inhibitor; a DNA-PK inhibitor; an IGF1R inhibitors; and a PLK inhibitor.

In various aspects of the invention, the disclosed treatment method comprises administering a mTOR inhibitor and a HDAC inhibitor. Exemplary combinations of mTOR inhibitors and HDAC inhibitors include, but are not limited to, rapamycin+domatinostat, rapamycin+givinostat, rapamycin+quisinostat, rapamycin+fimepinostat, rapamycin+panobinostat, rapamycin+belinostat. In a preferred aspect of the invention, the mTOR inhibitor is rapamycin and the HDAC inhibitor is domatinostat.

In various aspects of the invention, the disclosed treatment method comprises administering a mTOR inhibitor and a Hsp90 inhibitor. Exemplary combinations of mTOR inhibitors and Hsp90 inhibitors include, but are not limited to, rapamycin in combination with alvespimycin; rapamycin in combination with tanespimycin; rapamycin in combination with BIIB021; and rapamycin in combination with retaspimycin. In other aspects of the invention, the preferred mTOR inhibitor is rapamycin and the preferred Hsp90 inhibitor is alvespimycin.

In various aspects of the invention, the disclosed treatment method comprises administering a mTOR inhibitor and a NAD Biosynthesis and/or NAMPT inhibitor. Exemplary combinations of mTOR inhibitors and NAD Biosynthesis and/or NAMPT inhibitors include, but are not limited to, rapamycin in combination with daporinad; and rapamycin in combination with β-lapachone. In other aspects of the invention, the preferred mTOR inhibitor is rapamycin and the preferred NAD Biosynthesis and/or NAMPT inhibitor is daporinad.

In other aspects of the invention, the disclosed treatment method comprises administering a mTOR inhibitor and a Bcl-2 inhibitor. Exemplary combinations of mTOR inhibitors and Bcl-2 inhibitors include, but are not limited to, rapamycin in combination with navitoclax; and rapamycin in combination with obatoclax. In other aspects of the invention, the preferred mTOR inhibitor is rapamycin and the preferred Bcl-2 inhibitor is navitoclax.

In various aspects of the invention, the disclosed treatment method comprises administering a mTOR inhibitor and a MEK inhibitor. Exemplary combinations of mTOR inhibitors and MEK inhibitors include, but are not limited to, rapamycin in combination with PD0325901; rapamycin in combination with pimasertib; and rapamycin in combination with binimetinib. In other aspects of the invention, the preferred mTOR inhibitor is rapamycin and the preferred MEK inhibitor is PD0329501.

In various aspects of the invention, the disclosed treatment method comprises administering a Hsp90 inhibitor and a NAD Biosynthesis and/or NAMPT inhibitor. Exemplary combinations of Hsp90 inhibitors and NAD Biosynthesis and/or NAMPT inhibitors include, but are not limited to: alvespimycin in combination with daporinad; and alvespimycin in combination with β-lapachone. In other aspects of the invention, the preferred Hsp90 inhibitor is alvespimycin and the preferred NAD Biosynthesis and/or NAMPT inhibitor is daporinad.

In various aspects of the invention, the disclosed treatment method comprises administering a Hsp90 inhibitor and a HDAC inhibitor. Exemplary combinations of Hsp90 inhibitors and HDAC inhibitors include, but are not limited to, alvespimycin in combination with domatinostat; alvespimycin in combination with givinostat; alvespimycin in combination with quisinostat; alvespimycin in combination with fimepinostat; alvespimycin in combination with panobinostat; and alvespimycin in combination with belinostat. In other aspects of the invention, the preferred Hsp90 inhibitor is alvespimycin and the preferred HDAC inhibitor is domatinostat.

In various aspects of the invention, the disclosed treatment method comprises administering a HDAC inhibitor and a Bcl-2 inhibitor. Exemplary combinations of HDAC inhibitors and Bcl-2 inhibitors include, but are not limited to, navitoclax in combination with domatinostat; navitoclax in combination with givinostat; navitoclax in combination with quisinostat; navitoclax in combination with fimepinostat; navitoclax in combination with panobinostat; and navitoclax in combination with belinostat. In other aspects of the invention, the preferred Bcl-2 inhibitor is navitoclax and the preferred HDAC inhibitor is domatinostat.

In various aspects of the invention, the disclosed treatment method comprises administering a HDAC inhibitor and a MEK inhibitor. Exemplary combinations of HDAC inhibitors and MEK inhibitors include, but are not limited to, PD0325901 in combination with domatinostat; PD0325901 in combination with givinostat; PD0325901 in combination with quisinostat; PD0325901 in combination with fimepinostat; PD0325901 in combination with panobinostat; PD0325901 in combination with belinostat; pimasertib in combination with domatinostat; pimasertib in combination with givinostat; pimasertib in combination with quisinostat; pimasertib in combination with fimepinostat; pimasertib in combination with panobinostat; and pimasertib in combination with belinostat. In other aspects of the invention, the preferred MEK inhibitor is PD0325901 and the preferred HDAC inhibitor is domatinostat.

In various aspects of the invention, the disclosed treatment method comprises administering a HDAC inhibitor and a NAD Biosynthesis and/or NAMPT inhibitor. Exemplary combinations of HDAC inhibitors and NAD Biosynthesis and/or NAMPT inhibitors include, but are not limited to, daporinad in combination with domatinostat; daporinad in combination with givinostat; daporinad in combination with quisinostat; daporinad in combination with fimepinostat; daporinad in combination with panobinostat; and daporinad in combination with belinostat. In other aspects of the invention, the preferred NAD Biosynthesis and/or NAMPT inhibitor is daporinad and the preferred HDAC inhibitor is domatinostat.

In various aspects of the invention, the disclosed treatment method comprises administering a mTOR inhibitor and a Bromodomain (BET) inhibitor. Exemplary combinations of mTOR inhibitors and Bromodomain (BET) inhibitors include, but are not limited to, rapamycin and mivebresib, or rapamycin and AZD5153.

In various aspects of the invention, the disclosed treatment method comprises administering a HDAC inhibitor and a Bromodomain (BET) inhibitor. Exemplary combinations of HDAC inhibitors and Bromodomain (BET) inhibitors include, but are not limited to, domatinostat in combination with mivebresib; domatinostat in combination with AZD5153; panobinostat in combination with mivebresib; panobinostat in combination with AZD5153; belinostat in combination with mivebresib; and belinostat in combination with AZD5153.

In a further aspect, classes and specific examples of therapeutic agents useful with the disclosed treatment methods include, but are not limited to therapeutic combinations including, but not limited to drug classes, and specific drugs to be combined from these classes, listed in Table 3.

TABLE 3

| | Examples of Specific Drug |
|---|---|
| Examples of Drug Classes Suitable | Combinations in the Class |
| for Combinations | with Drugs with other Classes |
| mTOR inhibitors and HDAC inhibitors | Rapamycin + Domatinostat, |
| | Rapamycin + Givinostat, |
| | Rapamycin + Quisinostat, |
| | Rapamycin + Fimepinostat |
| | Rapamycin + Panobinostat |
| | Rapamycin + Belinostat |
| mTOR inhibitors and Hsp90 inhibitors | Rapamycin + Alvespimycin, |
| | Rapamycin + Tanespimycin, |
| | Rapamycin + BIIB021, |
| | Rapamycin + Retaspimycin |
| mTOR inhibitors and NAD Biosynthesis | Rapamycin + Daporinad |
| and/or NAMPT inhibitors | Rapamycin + β-Lapachone |
| mTOR inhibitors and Bcl-2 inhibitors | Rapamycin + Navitoclax, |
| | Rapamycin + Obatoclax |
| mTOR inhibitors and MEK inhibitors | Rapamycin + PD0325901, |
| | Rapamycin + Pimasertib, |
| | Rapamycin + Binimetinib |
| Hsp90 inhibitors and NAD Biosynthesis | Alvespimycin + Daporinad, |
| and/or NAMPT inhibitors | Alvespimycin + β-Lapachone |
| HDAC inhibitors and HSP90 inhibitors | Domatinostat + Alvespimycin |
| | Givinostat + Alvespimycin |
| | Quisinostat + Alvespimycin |
| | Fimepinostat + Alvespimycin |
| | Panobinostat + Alvespimycin |
| | Belinostat + Alvespimycin |
| HDAC inhibitors and Bcl-2 inhibitors | Domatinostat + Navitoclax |
| | Givinostat + Navitoclax |
| | Quisinostat + Navitoclax |
| | Fimepinostat + Navitoclax |
| | Panobinostat + Navitoclax |
| | Belinostat + Navitoclax |
| HDAC inhibitors and MEK inhibitors | Domatinostat + PD0325901 |
| | Givinostat + PD0325901 |
| | Quisinostat + PD0325901 |
| | Fimepinostat + PD0325901 |
| | Panobinostat + PD0325901 |
| | Belinostat + PD0325901 |
| | Domatinostat + Pimasertib |
| | Givinostat + Pimasertib |
| | Quisinostat + Pimasertib |
| | Fimepinostat + Pimasertib |
| | Panobinostat + Pimasertib |
| | Belinostat + Pimasertib |
| HDAC inhibitors and NAD Biosynthesis | Domatinostat + Daporinad, |
| and/or NAMPT inhibitors | Givinostat + Daporinad |
| | Quisinostat + Daporinad |
| | Fimepinostat + Daporinad |
| | Panobinostat + Daporinad |
| | Belinostat + Daporinad |
| mTOR inhibitors and Bromodomain | Rapamycin + Mivebresib, |
| (BET) inhibitors | Rapamycin + AZD5153 |
| HDAC and Bromodomain (BET) | Domatinostat + Mivebresib, |
| inhibitors | Domatinostat + AZD5153 |
| | Panobinostat + Mivebresib |
| | Panobinostat + AZD5153 |
| | Belinostat + Mivebresib |
| | Belinostat + AZD5153 |

In an aspect, in cancer models the therapeutic agents listed in the above tables can prevent the metastatic outgrowth of highly metastatic OS human and/or mouse and/or canine cancer cells in vitro and/or in other cancer types such as breast cancer and melanoma. These therapeutic agents have been tested in one or more of BME, SISgel and PuMA systems that maintains cancer cells in a dormant like state, and/or establish the expression of the Metastatic Endurance phenotype of low or highly metastatic cancer cells.

In an aspect, in cancer models the therapeutic agents listed in the above tables can prevent the metastatic outgrowth of highly metastatic OS human and/or mouse and/or canine cancer cells in vitro and/or in other cancer types such as breast cancer and melanoma. These therapeutic agents have been tested in one or more of BME, SISgel and PuMA systems that maintains cancer cells in a microscopic metastatic tumor like state, and/or establish the expression of the Metastatic Endurance phenotype of low or highly metastatic cancer cells.

In an aspect, therapeutic agents can be tested with any normal or modified ECM material (including SISgel and BME), as well as ex vivo assays (including PuMA) which has malignant or dormancy-phenotype suppressing activity, and/or which require or induce the ME phenotype in cancer cells. Mammalian tissue sources for ECMs are in general any tissue having an extracellular matrix that can be isolated from a mammal and decellularized. Thus, for example, most mammalian organs are tissue sources. The tissue sources can be for example any mammalian tissue, including but not limited to, the small intestine, large intestine, stomach, lung, liver, glands, kidney, pancreas, placenta, heart, bladder, and prostate, and any fetal tissue from any mammalian organ (e.g., umbilical cord).

### E. IN VITRO METHODS

In one aspect, disclosed are methods of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of an active pharmaceutical ingredient (API) selected from:
mTOR inhibitors;
heat shock protein 90 (HSP90) inhibitors;
heat shock protein 70 (HSP70) inhibitors;
unfolded protein response (UPR) inhibitors;
modulators of nicotinamide adenine dinucleotide (NAD+) metabolism;
a glutamyl-prolyl tRNA synthetase inhibitors;
glutaminase inhibitors;
poly(ADP)-ribose polymerase (PARP) inhibitors;
DNA-damaging agents;
agents capable of generating reactive oxygen species (ROS);
survivin inhibitors;
MEK 1 and/or MEK 2 inhibitors;
BCL-XL and/or BCL-2 inhibitors;
proteosome inhibitors;
muscarinic acetylcholine (mACh) antagonists;
B-Raf inhibitors;
Bromodomain (BET) protein inhibitors;
AXL kinase inhibitors;
NEDD8 inhibitors;
aurora kinase inhibitors;
histone deacetylase (HDAC) inhibitors (e.g., Class I selective histone deacetylase (HDAC) inhibitors);
PI3K inhibitors;
Akt inhibitors;
LSD inhibitors;
DNA-PK inhibitors;
IGF1R inhibitors; and
PLK inhibitors.

In one aspect, disclosed are methods of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of a class 1 selective HDAC inhibitor.

In one aspect, disclosed are methods of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of a drug selected from an active pharmaceutical ingredient (API), a large molecule, an antibody, a nucleic acid based drug, an aptamer, a peptide or a protein that inhibits the activity of epigenome modulators or regulators.

In one aspect, disclosed are methods of preventing or inhibiting proliferation of a dormant cancer cell, preventing or inhibiting the transitioning of a dormant cancer cell to a rapidly proliferating cancer call, or killing a dormant cancer cell, the method comprising contacting the dormant cancer cell with an effective amount of two or more active pharmaceutical ingredients (APIs) selected from:
a mTOR inhibitor;
a heat shock protein 90 (HSP90) inhibitor;
a heat shock protein 70 (HSP70) inhibitor;
an unfolded protein response (UPR) inhibitor;
a modulator of nicotinamide adenine dinucleotide (NAD+) metabolism;
a glutamyl-prolyl tRNA synthetase inhibitor;
a glutaminase inhibitor;
a apoly(ADP)-ribose polymerase (PARP) inhibitor;
DNA-damaging agent;
an agent capable of generating reactive oxygen species (ROS);
a survivin inhibitors;
a MEK 1 and/or MEK 2 inhibitor;
a Bcl-xL and/or Bcl-2 inhibitor;
a proteosome inhibitor;
a muscarinic acetylcholine (mACh) antagonist;
a B-Raf inhibitor;
a bromodomain (BET) protein inhibitor;
an AXL kinase inhibitor;
a NEDD8 inhibitor;
an aurora kinase inhibitor;
a histone deacetylase (HDAC) inhibitor;
a PI3K inhibitor;
an Akt inhibitor;
a LSD inhibitor;
a DNA-PK inhibitor;
an IGF1R inhibitor; and
a PLK inhibitor.

In a further aspect, the method comprises contacting the dormant cancer cell with an effective amount of exactly one of the APIs. In a still further aspect, the method comprises contacting the dormant cancer cell with an effective amount of at least two of the APIs.

In a further aspect, the drug is the API, and the API is an HDAC inhibitor.

In a further aspect, the drug is the API, and the API is domatinostat, givinostat, quisinostat, fimepinostat, panobinostat, belinostat, mivebresib, or AZD5153.

In a further aspect, the dormant cancer cell is a dormant disseminated tumor cell (DTC). In a still further aspect, the dormant cancer cell is a suppressed cancer cell. In yet a further aspect, the dormant cancer cell is a cancer stem cell. In an even further aspect, the dormant cancer cell is present in a reversible (quiescent) G0-G1 state. In a still further aspect, the dormant cancer cell is part of a micrometastasis.

In a further aspect, cellular proliferation is balanced by apoptosis.

In a further aspect, the dormant cancer cell has a metastatic endurance (ME) phenotype.

In a further aspect, the dormant cancer cell is selected from:
a sarcoma cell;
an osteosarcoma cell;
an osteogenic sarcoma cell;
a transitional cell;
a squamous cell;
a small cell carcinoma cell;
a medullary cell;
an adenocarcinoma cell; and
a basal cell carcinoma cell.

In a further aspect, the dormant cancer cell is selected from:
a bone cancer cell;
a bladder cancer cell;
a liver cancer cell;
a breast cancer cell;
a lung cancer cell;
a prostate cancer cell;
a pancreatic cancer cell;
a colon cancer cell;
a melanoma cell;
a brain cancer cell;
a kidney cancer cell;
a prostate cancer cell; and
a hematological cancer cell.

In a further aspect, the API is selected from rapamycin, ridaforolimus, temsirolimus, vistusertib, sapanisertib, everolimus, AZD8055, domatinostat, givinostat, quisinostat, fimepinostat, panobinostat, belinostat, mivebresib, AZD5153, telaglenastat, pevonedistat, BIIB021, alvespimycin, tanespimycin, retaspimycin, talazoparib, mitomycin C, alisertib, danusertib, (E)-daporinad, minnelide, halofuginone, PD0325901, picropodophyllin, oprozomib, β-lapachone, navitoclax, bemcentinib, triptolide, dabrafenib, pimasertib, YM-155, binimetinib, AZD7648, and tropicamide.

### F. METHODS OF TREATMENT

Disclosed are methods for the treatment of a patient afflicted with cancer or who may have dormant and/or micrometastatic and/or suppressed cancer cells or conditions characterized at least wherein such disease states or conditions may be treated by the administration of a therapeutically effective amount of a compound as described herein to a subject in need thereof.

Disclosed are methods for the treatment of a patient afflicted with cancer or who may have dormant and/or micrometastatic and/or suppressed cancer cells or conditions characterized at least wherein such disease states or conditions may be treated by the administration of a therapeutically effective amount of a combination of therapeutic agents along with or one or more therapeutic agents disclosed herein with other therapeutic agents, medical procedures including but not limited to surgery, radiation, and ablation, and/or alternative therapy approaches such as various life style approaches as described hereinabove to a subject in need thereof.

Disclosed are methods for the treatment of a patient afflicted with cancer or who may have microscopic metastatic tumor or conditions characterized at least wherein such disease states or conditions may be treated by the administration of a therapeutically effective amount of a combination of therapeutic agents along with or one or more therapeutic agents disclosed herein with other therapeutic agents, medical procedures including but not limited to surgery, radiation and ablation, and/or alternative therapy approaches such as various life style approaches as described hereinabove to a subject in need thereof.

Thus, in one aspect, disclosed are methods of preventing or inhibiting growth or proliferation of a microscopic metastatic tumor in a patient in need thereof, the method comprising contacting the microscopic metastatic tumor with an effective amount of one or more active pharmaceutical ingredients (APIs) selected from:
a mTOR inhibitor;
a heat shock protein 90 (HSP90) inhibitor;
a heat shock protein 70 (HSP70) inhibitor;
an unfolded protein response (UPR) inhibitor;
a modulator of nicotinamide adenine dinucleotide (NAD+) metabolism;
a glutamyl-prolyl tRNA synthetase inhibitor;
a glutaminase inhibitor;
a poly(ADP)-ribose polymerase (PARP) inhibitor;
a DNA-damaging agent;
an agent capable of generating reactive oxygen species (ROS);
a survivin inhibitors;
a MEK 1 and/or MEK 2 inhibitor;
a Bcl-xL and/or Bcl-2 inhibitor;
a proteosome inhibitor;
a muscarinic acetylcholine (mACh) antagonist;
a B-Raf inhibitor;
a BET protein inhibitor;
an AXL kinase inhibitor;
a NEDD8 inhibitor;
an aurora kinase inhibitor;
a histone deacetylase (HDAC) inhibitor;
a bromodomain (BET) inhibitor;
a PI3K inhibitors;
an Akt inhibitor;
a LSD inhibitor;
a DNA-PK inhibitor;
an IGF1R inhibitor; and
a PLK inhibitor.

As further disclosed herein, a patient in need of prevention or inhibition of growth or proliferation of a microscopic metastatic tumor means a patient previously diagnosed with any cancer that has a high risk for cancer recurrence or cancer progression as determined by one of a variety of different factors including, but not limited to, the type of cancer, the stage of detection, family history, genetic factors and biomarkers that a clinician may utilize for assessing such rish, such patient having received or not received prior treatment, and such patient currently receiving or not receiving treatment.

In some aspects of the disclosed method, the method comprises administering at least two APIs. In a further aspect, the two APIs are administered simultaneously. In a still further aspect, the two APIs are co-formulated. In yet a further aspect, the two APIs are administered sequentially.

In some aspects of the disclosed method, the microscopic metastatic tumor is one cell or more single cells single, or one or more clusters of multiple cells that have a metastatic endurance (ME) phenotype.

In some aspects of the disclosed method, the microscopic metastatic tumor is comprised of one or more cancer cells selected from:
sarcoma cells;
osteosarcoma cells;
osteogenic sarcoma cells;
transitional cells;
squamous cells;
small cell carcinoma cells;
medullary cells;
adenocarcinoma cells; and
basal cell carcinoma cells.

In some aspects of the disclosed method, the microscopic metastatic tumor is comprised of one or more cancer cells selected from:
bone cancer cells;
bladder cancer cells;
liver cancer cells;
breast cancer cells;
lung cancer cells;
prostate cancer cells;
pancreatic cancer cells;
colon cancer cells;
melanoma cells;
brain cancer cells;
kidney cancer cells;
prostate cancer cells; and
hematological cancer cells.

In some aspects of the disclosed method, the drug is selected from rapamycin, ridaforolimus, temsirolimus, vistusertib, sapanisertib, everolimus, AZD8055, domatinostat givinostat, quisinostat, fimepinostat, panobinostat, belinostat, mivebresib, AZD5153, telaglenastat, pevonedistat, BIIB021, alvespimycin, tanespimycin, retaspimycin, talazoparib, mitomycin C, alisertib, danusertib, (E)-daporinad, minnelide, halofuginone, PD0325901, picropodophyllin, oprozomib, β-lapachone, navitoclax, bemcentinib, triptolide, dabrafenib, pimasertib, YM-155, binimetinib, AZD7648, and tropicamide.

In one aspect, disclosed are methods of targeting minimal residual disease or preventing recurrence of cancer in a subject in need thereof, the method comprising administering to the subject an effective amount of an active pharmaceutical ingredient (API) selected from:
a mTOR inhibitor;
a heat shock protein 90 (HSP90) inhibitor;
a heat shock protein 70 (HSP70) inhibitor;
an unfolded protein response (UPR) inhibitor;
a modulator of nicotinamide adenine dinucleotide (NAD+) metabolism;
a glutamyl-prolyl tRNA synthetase inhibitor;
a glutaminase inhibitor;
a poly(ADP)-ribose polymerase (PARP) inhibitor;
a DNA-damaging agent;
an agent capable of generating reactive oxygen species (ROS);
a survivin inhibitor;
a MEK 1 and/or MEK 2 inhibitor;
a Bcl-xL and/or Bcl-2 inhibitor;
a proteosome inhibitor;
a muscarinic acetylcholine (mACh) antagonist;
a B-Raf inhibitor;
a bromodomain (BET) protein inhibitor;
an AXL kinase inhibitor;
a NEDD8 inhibitor;
an aurora kinase inhibitor;
a histone deacetylase (HDAC) inhibitor;
a PI3K inhibitor;
an Akt inhibitor;
a LSD inhibitor;
a DNA-PK inhibitor;
an IGF1R inhibitors; and
a PLK inhibitor.

In one aspect, disclosed are methods of targeting minimal residual disease or preventing recurrence of cancer in a subject in need thereof, the method comprising administering to the subject an effective amount of an active pharmaceutical ingredient (API), an antibody, or an immunotherapeutic that inhibits the activity of epigenome modulators or regulators.

As further disclosed herein, a subject in need of targeting minimal residual disease or prevention of recurrence of cancer means a patient previously diagnosed with cancer and having current minimal residual disease, or a patient diagnosed with cancer and with undetected minimal residual disease, such patient being at high risk for cancer recurrence or cancer progression as determined by one of a variety of different factors including, but not limited to, the type of cancer, the stage of detection, family history, genetic factors and biomarkers that a clinician may utilize for assessing such risk, such patient having received or not received prior treatment, and such patient currently receiving or not receiving treatment.

In some aspects of the disclosed method, the drug is the API and the API is an HDAC inhibitor.

In some aspects of the disclosed method, the drug is the API and the API is domatinostat, givinostat, quisinostat, fimepinostat, panobinostat, belinostat, mivebresib, or AZD5153.

In some aspects of the disclosed method, the drug exposure or dosing regimen of the API is effective against the dormant cancer cells at dosing regimen or exposure levels that are expected to be tolerable to the subject as predicted using the Clinical Dose Selectivity Index (CDSI). In some aspects of the disclosed method, the drug exposure or dosing regimen of the API is effective against the dormant cancer cells at dosing regimen or exposure levels that are expected to be tolerable to the subject as predicted using reference to established safety and tolerability profiles in human subjects including but not limited to maximum tolerated dose studies and pharmacokinetic data. In some aspects of the disclosed method, the drug exposure or dosing regimen of the API is expected to be tolerable to the subject as predicted using the Clinical Dose Selectivity Index (CDSI). In some aspects of the disclosed method, the drug exposure or dosing regimen of the API is predicted using reference to established safety and tolerability profiles in human subjects.

In some aspects of the disclosed method, the subject is in remission after having undergone treatment for bone cancer, bladder cancer, liver cancer, breast cancer, lung cancer, prostate cancer, pancreatic cancer, colon cancer, melanoma, brain cancer, kidney cancer, prostate cancer, or hematological cancer.

In some aspects of the disclosed method, the subject has been identified as having a high risk for the development or recurrence of bone cancer, bladder cancer, liver cancer, breast cancer, lung cancer, prostate cancer, pancreatic cancer, colon cancer, melanoma, brain cancer, kidney cancer, prostate cancer, or hematological cancer, such patient being a patient previously diagnosed with any cancer with a high risk for cancer recurrence or cancer progression as determined by one of a variety of different factors including, but not limited to, the type of cancer, the stage of detection, family history, genetic factors and biomarkers that a clinician may utilize for assessing such risk, such patient with or without prior treatment, with or without current treatment.

In some aspects of the disclosed method, the subject has been identified as having a high risk based on detection of a threshold amount of one or more selected from circulating tumor cells (CTCs), circulating tumor DNA or cell-free DNA, and a genetic or non-genetic biomarker indicating an elevated risk of cancer development or recurrence, wherein the threshold amount is detected in a biological sample taken from the subject.

In some aspects of the disclosed method, the API is administered to the subject in a neoadjuvant or adjuvant setting.

In some aspects of the disclosed method, the API is administered to the subject on an ongoing basis.

In some aspects of the disclosed method, the API is selected from rapamycin, ridaforolimus, temsirolimus, vistusertib, sapanisertib, everolimus, AZD8055, domatinostat, givinostat, quisinostat, fimepinostat, mivebresib, AZD5153, telaglenastat, pevonedistat, BIIB021, alvespimycin, tanespimycin, retaspimycin, talazoparib, mitomycin C, alisertib, danusertib, (E)-daporinad, minnelide, halofuginone, PD0325901, picropodophyllin, oprozomib, B-lapachone, navitoclax, bemcentinib, triptolide, dabrafenib, pimasertib, YM-155, binimetinib, AZD7648, and tropicamide. In some aspects of the disclosed method, the API is domatinostat, givinostat, quisinostat, fimepinostat, panobinostat, belinostat, mivebresib, or AZD5153.

In some aspects of the disclosed method, the method comprises administering to the subject an effective amount of exactly one API. In some aspects of the disclosed method, the method comprises administering to the subject patient an effective amount of two or more of the APIs.

In some aspects of the disclosed method, the minimal residual disease is comprised of one or more cancer cells selected from:
sarcoma cells;
osteosarcoma cells;
osteogenic sarcoma cells;
transitional cells;
squamous cells;
small cell carcinoma cells;
medullary cells;
adenocarcinoma cells; and
basal cell carcinoma cells.

In some aspects of the disclosed method, the microscopic metastatic tumor microscopic metastatic tumor is comprised of one or more cancer cells selected from:
bone cancer cells;
bladder cancer cells;
liver cancer cells;
breast cancer cells;
lung cancer cells;
prostate cancer cells;
pancreatic cancer cells;
colon cancer cells;
melanoma cells;
brain cancer cells;
kidney cancer cells;
prostate cancer cells; and
hematological cancer cells.

In some aspects of the disclosed method, each of the two or more APIs is independently selected from rapamycin, ridaforolimus, temsirolimus, vistusertib, sapanisertib, everolimus, AZD8055, domatinostat, givinostat, quisinostat, fimepinostat, panobinostat, belinostat, mivebresib, AZD5153, telaglenastat, pevonedistat, BIIB021, alvespimycin, tanespimycin, retaspimycin, talazoparib, mitomycin C, alisertib, danusertib, (E)-daporinad, minnelide, halofuginone, PD0325901, picropodophyllin, oprozomib, β-lapachone, navitoclax, bemcentinib, triptolide, dabrafenib, pimasertib, YM-155, binimetinib, AZD7648, and tropicamide.

In some aspects of the disclosed method, the subject has been diagnosed as having an overt metastases. In some aspects of the disclosed method, the subject has been diagnosed as being in partial or complete remission. In some aspects of the disclosed method, the subject has been diagnosed as having a resectable osteosarcoma metastases. In some aspects of the disclosed method, the subject has been diagnosed as having an unresectable osteosarcoma metastases. In some aspects of the disclosed method, the subject has been diagnosed as having a resectable primary osteosarcoma. In some aspects of the disclosed method, the subject has been diagnosed as having an unresectable resectable primary osteosarcoma. In some aspects of the disclosed method, the subject has been diagnosed as having a resectable primary osteosarcoma prior to detection of metastases. In some aspects of the disclosed method, the subject has been diagnosed as having a resectable primary osteosarcoma upon or after diagnosis of metastatic cancer before resection of primary tumor and continuing after resection. In some aspects of the disclosed method, the subject has been diagnosed as having a resectable primary osteosarcoma prior to detection of metastases. In some aspects of the disclosed method, the subject has been diagnosed as having a resectable primary osteosarcoma prior to detection of metastases. In some aspects of the disclosed method, the subject has been diagnosed as having a resectable primary osteosarcoma upon or after diagnosis of metastatic cancer before resection of primary tumors and continuing after in order to prevent an initial or subsequent recurrences resection.

In some aspects of the disclosed method, the method is used in conjunction with ctDNA liquid biopsy to inform dose and regimen in a subject having cancer without evidence of disease.

In some aspects of the disclosed method, the target whole blood concentration range is:
from about 0.5 nM or greater for rapamycin;
from about 100 nM or greater for domatinositat;
from about 0.5 nM or greater for daporinad;
from about 10 nM or greater for alvespimycin;
from about 500 nM or greater for navitoclax;
from about 1 nM or greater for PD0325901;
from about 100 nM or greater for pimasertib;
from about 400 nM or greater for panobinostat;
from about 160 nM or greater for belinostat;
from about 400 nM or greater for givinostat;
from about 10 nM or greater for quisinostat; or
from about 5 nM to about 10 nM or from about 10 nM to about 10,000 nM for fimepinostat.

In some aspects of the disclosed method, the API is:
rapamycin at 0.5, 1, 1.5, 2, 2.5, 3, 3.5, 4, 4.5, 5, 6, 7, 8, 9, or 10 mg, given once or twice a day;
daporinad administered every 4 weeks as a continuous intravenous infusion over 96 hours at 0.018 mg/m²/h, 0.036 mg/m²/h, 0.072 mg/m²/h, 0.108 mg/m²/h, or 0.126 mg/m²/h;
domatinostat at 100, 150, 200, 250, 300, 350, 400, 450, 500 or 600 mg, given once or twice a day; or
alvespimycin at 10, 15, 20, 25, 30, 35, or 40 mg, given once or twice a day, or every 2 days or 3 days.

In some aspects of the disclosed method, the two or more APIs are:
rapamycin and domatinostat;
rapamycin and daporinad;
domatinostat and daporinad;
rapamcyin and alvespimycin;
domatinostat and alvespimycin; or
alvespimycin and daporinad.

In some aspects of the disclosed method, the two or more APIs are:
rapamycin and domatinostat, wherein rapamycin is administered at an amount of 0.5 to 10 mg, given once or twice a day, and wherein domatinostat is administered at an amount of 100 to 600 mg per day, given once or twice a day;
rapamycin and daporinad, wherein rapamycin is administered at an amount of 0.5 to 10, given once or twice a day, and wherein daporinad is administered intravenously at an amount of 0.018 mg/m²/h to 0.126 mg/m²/h for one or more days;
domatinostat and daporinad, wherein domatinostat is administered at an amount of 100 to 600 mg, given once or twice a day, and wherein daporinad is administered intravenously at an amount of 0.018 mg/m²/h to 0.126 mg/m²/h;
rapamcyin and alvespimycin, wherein rapamycin is administered at an amount of 0.5 to 10 mg, given once or twice a day, and wherein alvespimycin is administered at an amount of 10-40 mg, given once or twice a day or every 2 days or 3 days;
domatinostat and alvespimycin, wherein domatinostat is administered at an amount of 100 to 600 mg, given once or twice a day, and wherein alvespimycin is administered at an amount of 10-40 mg, given once or twice a day or every 2 days or 3 days; or
alvespimycin and daporinad, wherein alvespimycin is administered at an amount of 10-40 mg, given once or twice a day or every 2 days or 3 days, and wherein daporinad is administered intravenously at an amount of 0.018 mg/m²/h to 0.126 mg/m²/h for one or more days.

In some aspects of the disclosed method, each API is administered separately. In some aspects of the disclosed method, each API is administered sequentially. In some aspects of the disclosed method, each API is administered simultaneously.

In some aspects of the disclosed method, the two or more APIs are:
rapamycin and domatinostat, wherein rapamycin is administered at an amount of about 0.5, 1, 1.5, 2, 2.5, 3, 3.5, 4, 4.5, 5, 6, 7, 8, 9, or 10 mg, given once or twice a day, and wherein domatinostat is administered at an amount of about 100, 150, 200, 250, 300, 350, 400, 450, or 500 mg, given once or twice a day;
rapamycin and daporinad, wherein rapamycin is administered at an amount of about 0.5, 1, 1.5, 2, 2.5, 3, 3.5, 4, 4.5, 5, 6, 7, 8, 9, or 10 mg, given once or twice a day, and wherein daporinad is administered intravenously at an amount of about 0.018 mg/m²/h, 0.036 mg/m²/h, 0.072 mg/m²/h, 0.108 mg/m²/h, or 0.126 mg/m²/h;
domatinostat and daporinad, wherein domatinostat is administered at an amount of about 100, 150, 200, 250, 300, 350, 400, 450, or 500 mg, given once or twice a day, and wherein daporinad is administered intravenously at an amount of about 0.018 mg/m²/h, 0.036 mg/m²/h, 0.072 mg/m²/h, 0.108 mg/m²/h, or 0.126 mg/m²/h;
rapamcyin and alvespimycin, wherein rapamycin is administered at an amount of about 0.5, 1, 1.5, 2, 2.5, 3, 3.5, 4, 4.5, 5, 6, 7, 8, 9, or 10 mg, given once or twice a day, and wherein alvespimycin is administered at an amount of about 10, 15, 20, 25, 30, 35, or 40 mg, given once or twice a day or every 2 days or 3 days;
domatinostat and alvespimycin, wherein domatinostat is administered at an amount of about 100, 150, 200, 250, 300, 350, 400, 450, or 500 mg, given once or twice a day, and wherein alvespimycin is administered at an amount of about 10, 15, 20, 25, 30, 35, or 40 mg, given once or twice a day or every 2 days or 3 days;
alvespimycin and daporinad, wherein alvespimycin is administered at an amount of about 10, 15, 20, 25, 30, 35, or 40 mg, given once or twice a day or every 2 days or 3 days, and wherein daporinad is administered every 4 weeks as a continuous intravenous infusion over 96 hours at about 0.018 mg/m²/h, 0.036 mg/m²/h, 0.072 mg/m²/h, 0.108 mg/m²/h, or 0.126 mg/m²/h; or
alvespimycin and daporinad, wherein daporinad is administered every 4 weeks as a continuous intravenous infusion over 96 hours at about 0.018 mg/m²/h, 0.036 mg/m²/h, 0.072 mg/m²/h, 0.108 mg/m²/h, or 0.126 mg/m²/h.

In some aspects of the disclosed method, the two or more APIs are administered for a first time period of up to 1, 2, 3, 4, 6, 12, or 24 months.—In some aspects of the disclosed method, after the first time period, only one of the two or more APIs is administered for a second time period.

In some aspects of the disclosed method, the two or more APIs are domatinostat and rapamycin, wherein each of domatinostat and rapamycin is administered for a first time period, and wherein after the first time period only one of domatinostat and rapamycin is administered for a second time period. In some aspects of the disclosed method, each of domatinostat and rapamycin is administered for a first time period of up to one month and thereafter only one of domatinostat and rapamycin is administered for the second time period. In some aspects of the disclosed method, each of domatinostat and rapamycin is administered for a first time period of up to two months and thereafter only one of domatinostat and rapamycin is administered for the second time period. In some aspects of the disclosed method, each of domatinostat and rapamycin is administered for a first time period of up to three months and thereafter only one of domatinostat and rapamycin is administered for the second time period. In some aspects of the disclosed method, each of domatinostat and rapamycin is administered for a first time period of up to four months and thereafter only one of domatinostat and rapamycin is administered for the second time period. In some aspects of the disclosed method, each of domatinostat and rapamycin is administered for a first time period of up to six months and thereafter only one of domatinostat and rapamycin is administered for the second time period. In some aspects of the disclosed method, each of domatinostat and rapamycin is administered for a first time period of up to twelve months and thereafter only one of domatinostat and rapamycin is administered for the second time period. In some aspects of the disclosed method, each of domatinostat and rapamycin is administered for a first time period of up to twenty-four months and thereafter only one of domatinostat and rapamycin is administered for the second time period.

A therapeutically effective amount of a compound refers to an amount that is effective in controlling, reducing, or suppressing the growth or expression of dormant cancer cells, or microscopic metastatic tumor. Currently, the ability to determine the direct effect on a dormant cancer cells, or microscopic metastatic tumors, or minimal residual disease is limited and challenging due to the complexities and limitations of diagnostic procedures to assess the state of such microscopic biospecimen in a living patient. It is possible that future imaging, liquid biopsy, or other analysis will enable improved ways to determine such effects. Currently, an assessment of such effects will require correlative science approaches for assessing various biomarkers related to metastatic endurance and other aspects of cancer progression using biospecimen collected from patients. This may include assessing ctDNA, or assessing blood, urine, or tumor samples biomarkers related to tumor growth, activity or metastatic endurance. In the case of assessing ctDNA, a liquid biopsy can be collected every few weeks from a subject without detectable disease and the level of ctDNA related to the cancer assessed to determine if it is reduced or attenuated compared to patients that are not treated, or compared to patients in which recurrence of the cancer is observed. ctDNA biomarker assessment can be used to assess and inform dosing regimens of a drug and use of a drug or drug combinations of the invention. The dosing regimen and/or APIs and/or API combinations can be tailored to cause a decrease in ctDNA biomarkers, or maintaining ctDNA levels or slowing the increase in their levels compared to untreated patients or patients that have disease recurrence or progression. A similar assessment of a drug or drug combinations of the invention or informing dosing regimens of a drug and use of a drug, or drug combinations of the invention cab be also based on other biomarkers detected in the blood, or urine that reflect the presence, amount or activity of dormant cancer cells, or microscopic metastatic tumors, or minimal residual disease.

A patient or subject in need is a patient previously diagnosed with any cancer with a high risk for cancer recurrence or cancer progression as determined by one of a variety of different factors including, but not limited to, the type of cancer, the stage of detection, family history, genetic factors and biomarkers that a clinician may utilize for assessing such risk. Such patient may have been diagnosed and treated previously for primary and/or metastatic cancer. Such patient may have been previously treated, and treatment stopped after a successful course of treatment, or for lack of efficacy or for toxicity or tolerability issues. Such patient may be currently on treatment with other drugs, radiation or other interventions. Such patient may have not been previously treated or may have been treated inadequately, including using API or combination disclosed in this invention. Inadequate treatment includes incorrect dosing schedules, insufficient treatment timespans, lack of sufficient combinations, drug holidays, or stopping treatment, even when a subject received a disclosed API or disclosed combination.

The ability to determine the direct effect on a dormant cancer cells, or microscopic metastatic tumors can be based on clinical outcomes when treating patients with resectable cancer, when there is a high likelihood that such dormant cancer cells, or microscopic metastatic tumors, or minimal residual disease, are present and will cause detectable recurrence unless dormant cancer cells, or microscopic metastatic tumors, or minimal residual disease are controlled, reduced, or suppressed in their ability to grow. Osteosarcoma (OS) is cancer that provides a unique opportunity to assess the ability of a treatment to cause such effects using biomarkers as well as clinical outcomes such as cancer progression. OS most commonly affects children, adolescents, and young adults and is the primary malignant bone tumor. OS tends to occur in the metaphysis of long bones, and most commonly occurs in the distal femur (43%), proximal tibia (23%), or humerus (10%). More than 85% of metastatic disease occurs in the lung. Until about 35 years ago, standard of care for treating a patient diagnosed with localized primary OS tumors involved amputation or salvage surgery that removes the entire tumor. Nevertheless, in 80% of the patients the cancer returned within a few years, almost always in the lung, indicating that dormant cancer cells, or microscopic metastatic tumors originating in the primary tumor are causing the recurrences, which are deadly in the large majority of cases. Once MAP chemotherapy (Metbot+Doxorubiein+Cisplatin) was introduced and became standard of care for treating primary osteosarcoma tumors about 35 years ago, the rate of recurrence was reduced to about 35-40%, typically within two years, with dismal survival rates for those who recur. The recurrence post-surgery and MAP therapy is attributed to dormant cancer cells, or microscopic metastatic tumors. For osteosarcoma patients who recur in one or both lungs, treatments and outcomes are based on the possibility of surgically resecting the detectable metastatic tumors. Such patients are treated with various chemotherapy drugs such as Ifosfamide (or cyclophosphamide) at standard or high dose alone or in combination with etoposide, and gemcitabine/docetaxel, which have all been employed for patients with osteosarcoma at the time of recurrence. Recently, multi-kinase inhibitors such as regorafenib (Stivarga), sorafenib (Nexavar), or cabozantinib (Cabometyx) have been added at treatment options. All of these drugs may have some limited efficacy in increasing PFS, but are far from curative and have little if any benefit on survival. For patients with non-resectable lung metastases, outcomes are worse than for patients with fully resectable lung tumors. Resectable patients are treated with similar drugs and have an overall event free survival (EFS) of approximately 20% at 12 months following surgical removal of all metastatic lesions.

The ability to determine the direct effect on a dormant cancer cells, or microscopic metastatic tumors can be based on clinical effects or outcomes when treating patients with resectable cancer, in which there is a high likelihood that such dormant cancer cells, and, or microscopic metastatic tumors are present and will cause detectable recurrence unless such dormant cancer cells, or microscopic metastatic tumors are controlled, reduced, or suppressed in their ability to grow.

In a preferred embodiment of the invention, treatment using methods of the invention will start as soon as possible after diagnosis of a metastatic tumor in a cancer patient. Such a tumor, or multiple tumors, may be resectable, or non-resectable, as determined by a surgeon who is skilled in the art, with the aid of imaging methods and other diagnostic methods and based on various assessment utilized in practice for each given cancer type as practiced by one skilled in the art. For certain cancers, treatment may be initiated and be effective even if initiated many months, or years after initial diagnosis, especially in situation when the patient is in remission. As examples, breast cancer patients often remain in remission for years and even decades before metastatic recurrence; patients with primary colon cancer and cervical cancer can recur years later; in melanoma, more than 6% of all patients recur 10 or more years after initial diagnosis. In a preferred embodiment of the invention, treatment using methods of the invention will start before surgical removal of one or more resectable metastatic tumors. In a preferred embodiment of the invention, treatment using methods of the invention will start within 1 week of surgical removal of one or more resectable metastatic tumors. In yet another embodiment of the invention, treatment using methods of the invention will start within 2 weeks of surgical removal of one or more resectable metastatic tumors. In yet another embodiment of the invention, treatment using methods of the invention will start within 1 month of surgical removal of one or more resectable metastatic tumors. In yet another embodiment of the invention, treatment using methods of the invention will start within 2 months of surgical removal of one or more resectable metastatic tumors. In yet another embodiment of the invention, treatment using methods of the invention will start within one month of surgical removal of one or more resectable metastatic tumors. In yet another embodiment of the invention, treatment using methods of the invention will start within 4 months of surgical removal of one or more resectable metastatic tumors. In yet another embodiment of the invention, treatment using methods of the invention will start within 6 months of surgical removal of one or more resectable metastatic tumors. In yet another embodiment of the invention, treatment using methods of the invention will start within 12 months of surgical removal of one or more resectable metastatic tumors. In yet another embodiment of the invention, treatment using methods of the invention will start within 24 months of surgical removal of one or more resectable metastatic tumors. In yet another embodiment of the invention, treatment using methods of the invention will start within 48 months of surgical removal of one or more resectable metastatic tumors. In yet another embodiment of the invention, treatment using methods of the invention will start within 96 months of surgical removal of one or more resectable metastatic tumors. In yet another embodiment of the invention, treatment using methods of the invention will start within 128 months of surgical removal of one or more resectable metastatic tumors. In yet another embodiment of the invention, treatment using methods of the invention will start within 256 months of surgical removal of one or more resectable metastatic tumors.

In a preferred embodiment of the invention, treatment using methods of the invention will be applied to a patient or subject in need who has metastatic osteosarcoma (OS). In such patient, a tumor, or multiple tumors may be resectable, or non-resectable, as determined by a surgeon who is skilled in the art, with the aid of imaging methods and other diagnostic methods and based on various assessment utilized in practice for each given cancer type as practiced by one skilled in the art. Fully resectable OS patients generally have a 80% or so chance to have additional metastatic recurrences within about 12 months of diagnosis and/or progression of non resectable tumors within several months and therefore treating them using methods of the invention to prevent recurrence is warranted. In a most preferred embodiment of the invention, treatment using methods of the invention will be applied to a patient or subject in need who has resectable metastatic OS in one or both lungs. Non-resectable OS patients generally have a 88% or so chance to have disease progression at 4 months after diagnosis of metastatic cancer, and therefore treating them using methods of the invention to prevent recurrence is warranted.

In a preferred embodiment of the invention, treatment using methods of the invention will start as soon as possible after diagnosis of metastatic osteosarcoma that is fully resectable. Such a tumor may be deemed resectable by a surgeon who is skilled in the art, with the aid of imaging methods and other diagnostic methods and based on various assessment utilized in practice by one skilled in the art. In a preferred embodiment of the invention, treatment using methods of the invention will start before surgical removal of one or more resectable metastatic tumors. In a preferred embodiment of the invention, treatment using methods of the invention will start within 1 week of surgical removal of one or more resectable metastatic tumors. In yet another embodiment of the invention, treatment using methods of the invention will start within 2 weeks of surgical removal of one or more resectable metastatic tumors. In yet another embodiment of the invention, treatment using methods of the invention will start within 1 month of surgical removal of one or more resectable metastatic tumors. In yet another embodiment of the invention, treatment using methods of the invention will start within 2 months of surgical removal of one or more resectable metastatic tumors. In yet another embodiment of the invention, treatment using methods of the invention will start within one month of surgical removal of one or more resectable metastatic tumors. In yet another embodiment of the invention, treatment using methods of the invention will start within 4 months of surgical removal of one or more resectable metastatic tumors. In yet another embodiment of the invention, treatment using methods of the invention will start within 6 months of surgical removal of one or more resectable metastatic tumors. In yet another embodiment of the invention, treatment using methods of the invention will start within 12 months of surgical removal of one or more resectable metastatic tumors.

In a preferred embodiment of the invention, treatment using methods of the invention will be applied to a patient or subject in need who has non-resectable metastatic OS in one or both lungs. In a preferred embodiment of the invention, treatment using methods of the invention will start as soon as possible after diagnosis of metastatic osteosarcoma that includes one or more tumors that are non-resectable. Such a tumor may be deemed non-resectable by a surgeon who is skilled in the art, with the aid of imaging methods and other diagnostic methods and based on various assessment utilized in practice by one skilled in the art. In a preferred embodiment of the invention, treatment using methods of the invention will start within 1 week of diagnosis of the non-resectable metastatic OS tumors. In yet another embodiment of the invention, treatment using methods of the invention will start within 2 weeks of diagnosis of the non-resectable metastatic OS tumors. In yet another embodiment of the invention, treatment using methods of the invention will start within 4 weeks of diagnosis of the non-resectable metastatic OS tumors. In yet another embodiment of the invention, treatment using methods of the invention will start within 8 weeks of diagnosis of the non-resectable metastatic OS tumors. In yet another embodiment of the invention, treatment using methods of the invention will start within 12 weeks of diagnosis of the non-resectable metastatic OS tumors. In yet another embodiment of the invention, treatment using methods of the invention will start within 16 weeks of diagnosis of the non-resectable metastatic OS tumors. In yet another embodiment of the invention, treatment using methods of the invention will start within 6 months of diagnosis of the non-resectable metastatic OS tumors. In yet another embodiment of the invention, treatment using methods of the invention will start within 12 months of diagnosis of the non-resectable metastatic OS tumors.

A patient or subject in need may be an OS patient diagnosed with a primary tumor, without diagnosis of a metastatic tumor. Such OS patients generally have a 35% or so chance to have a metastatic recurrence within about 24 months and therefore treating them using methods of the invention to prevent recurrence is warranted. In preferred embodiments of the invention as described below, the treatments refer to treatments using methods of the invention in patients that have not recurred, with the purpose of preventing an initial metastatic recurrence, limit the number of recurrences, the extent of the recurrence or the extent of subsequent recurrences should an initial recurrence occur. In the most preferred embodiment of the invention, treatment using methods of the invention will start as soon as possible after diagnosis of the primary tumor. In a preferred embodiment of the invention, treatment using methods of the invention will start before surgical removal of the primary tumor. In a preferred embodiment of the invention, treatment using methods of the invention will start within 1 week of surgical removal of the primary tumor. In yet another embodiment of the invention, treatment using methods of the invention will start within 2 weeks of surgical removal of the primary tumor. In yet another embodiment of the invention, treatment using methods of the invention will start within 1 month of surgical removal of the primary tumor. In yet another embodiment of the invention, treatment using methods of the invention will start within 2 months of surgical removal of the primary tumor. In yet another embodiment of the invention, treatment using methods of the invention will start within one month of surgical removal of the primary tumor. In yet another embodiment of the invention, treatment using methods of the invention will start within 4 months of surgical removal of the primary tumor. In yet another embodiment of the invention, treatment using methods of the invention will start within 6 months of surgical removal of the primary tumor. In yet another embodiment of the invention, treatment using methods of the invention will start within 12 months of surgical removal of the primary tumor. In yet another embodiment of the invention, treatment using methods of the invention will start within 18 months of surgical removal of the primary tumor. In yet another embodiment of the invention, treatment using methods of the invention will start within 24 months of surgical removal of the primary tumor. In yet another embodiment of the invention, treatment using methods of the invention will start within 36 months of surgical removal of the primary tumor. In yet another embodiment of the invention, treatment using methods of the invention will start within 48 months of surgical removal of the primary tumor. In yet another embodiment of the invention, treatment using methods of the invention will start within 60 months of surgical removal of the primary tumor.

Several clinical trial designs can be utilized to assess if a treatment is effective in controlling, reducing, or suppressing the growth or expression of dormant cancer cells, or microscopic metastatic tumors. The assessment of such effect can utilize clinical endpoints such as progression free survival, event free survival or overall survival.

Various clinical trial designs can be utilized to assess if a method of treatment of the invention is effective in controlling, reducing, or suppressing the growth of undetected dormant cells, microscopic metastatic tumors, or undetected minimal residual disease in osteosarcoma patients with fully resectable lung metastases who are in complete remission. Such trial designs include assessing event free survival (EFS) at some time point, such as 12 months following diagnosis of one or more non-resectable metastatic tumors and therapeutic treatment, with the goal of increasing EFS. Such a study can be an open label study or a controlled study comparing treatment to a cohort on standard of care or physician's choice alone or to standard of care or physician's choice on top of the assessed treatment. An increase in EFS will indicate that the method of treatment of the invention may be effective in controlling, reducing, or suppressing the growth of undetected dormant cells, microscopic metastatic tumors, or undetected minimal residual disease in in osteosarcoma patients. As an example, a trial can be designed to demonstrate the ability of treatment to extend the EFS beyond the expected 20% based on well-established historical data at 12-months post diagnosis, wherein an increase in EFS will indicate that the method of treatment of the invention may be effective in controlling, reducing, or suppressing the growth of undetected dormant cells, microscopic metastatic tumors, or undetected minimal residual disease in in osteosarcoma patients.

Another clinical trial design that can be utilized to assess if a method of treatment of the invention is effective in controlling, reducing, or suppressing the growth of undetected dormant cells, microscopic metastatic tumors, or undetected minimal residual disease in osteosarcoma patients with fully resectable primary tumor without detectable metastatic tumors who are in complete remission involves an end point of first metastatic recurrence in the lung or elsewhere. In osteosarcoma, recurrence in such resectable patients on standard of care is about 35% at 24 months post diagnosis. A decrease in recurrence rates will indicate that the method of treatment of the invention may be effective in controlling, reducing, or suppressing the growth undetected dormant cells, microscopic metastatic tumors, or undetected minimal residual disease in osteosarcoma patients.

Another clinical trial design that can be utilized to assess if a method of treatment of the invention is effective in controlling, reducing, or suppressing the growth of undetected dormant cells, microscopic metastatic tumors, or undetected minimal residual disease in osteosarcoma patients with fully resectable primary tumor without detectable metastatic tumors who are in complete remission involves an end point of second metastatic recurrence in the lung or elsewhere. In osteosarcoma, while first recurrence in such resectable patients on standard of care is about 35% at 24 months post diagnosis, about 80% will have one or more additional recurrence within a year post diagnosis of the initial recurrence. Treating patients before an initial recurrence and including an end point of a second recurrence and reducing the rate of second recurrence rates will indicate that the method of treatment of the invention may be effective in controlling, reducing, or suppressing the growth undetected dormant cells, microscopic metastatic tumors, or undetected minimal residual disease in osteosarcoma patients.

Another clinical trial design that can be utilized to assess if a method of treatment of the invention is effective in controlling, reducing, or suppressing the growth of undetected dormant cells, microscopic metastatic tumors, or undetected minimal residual disease in osteosarcoma patients with fully resectable lung metastases who are in complete remission involves an end point of overall survival. In osteosarcoma, overall survival of such resectable patients is about 50% at 26 months post diagnosis of a resectable lung metastasis. An increase in overall survival will indicate that the method of treatment of the invention may be effective in controlling, reducing, or suppressing the growth undetected dormant cells, microscopic metastatic tumors, or undetected minimal residual disease in osteosarcoma patients.

Yet another clinical trial design can be utilized to assess if a method of treatment of the invention is effective in controlling, reducing, or suppressing the growth of detectable tumors or minimal residual disease in osteosarcoma patients. Such trial designs include assessing progression free survival (PFS) at four months following diagnosis of one or more non-resectable metastatic tumors and therapeutic treatment, with the goal of increasing PFS beyond the expected 12% at 4-months post treatment. Such a study can be an open label study or a controlled study comparing treatment to a cohort on standard of care or physician's choice alone or to standard of care or physician's choice on top of the assessed treatment. An increase in PFS will indicate that the method of treatment of the invention may be effective in controlling, reducing, or suppressing the growth of detectable tumors or minimal residual disease in osteosarcoma patients.

Another clinical trial design that can be utilized to assess if a method of treatment of the invention is effective in controlling, reducing, or suppressing the growth of undetected dormant cells, microscopic metastatic tumors, or undetected minimal residual disease in triple negative breast cancer (TNBC) patients who are in complete remission post standard of care involves an end point of metastatic recurrence. In such TNBC patients, recurrence rates are about 25% at 36 months post treatment. A decrease in recurrence rates will indicate that the method of treatment of the invention may be effective in controlling, reducing, or suppressing the growth undetected dormant cells, microscopic metastatic tumors, or undetected minimal residual disease in TNBC patients.

Thus, in various aspects, the method comprises administering a therapeutically effective amount of rapamycin. The therapeutically effective amount of rapamycin can be determined by methods known to one of skill in the art or as described elsewhere herein. Thus, in various aspects, the therapeutically effective amount of rapamycin is of from about 0.1 mg to about 1 mg, about 1 mg to about 2 mg, or about 2 mg to about 5 mg, or about 5 mg to about 15 mg. In a further aspect, rapamycin is administered in an amount of about 15 mg to about 50 mg. In various further aspects, rapamycin is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for rapamycin is of from about 100 pM to about 1 nM, about 1 nM to about 5 nM, or about 5 nM to about 10 nM, or about 10 nM to about 20 nM. In various further aspects, the target whole blood concentration for rapamycin is about 20 nM to about 1 uM.

As would be understood by one of ordinary skill in the art, the whole blood concentration is determined by analyzing the pharmacokinetics of rapamycin (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of domatinostat. The therapeutically effective amount of domatinostat can be determined by methods known to one of skill in the art or as described elsewhere herein. Thus, in various aspects, the therapeutically effective amount of domatinostat is of from about 50 mg to about 100 mg, about 100 mg to about 200 mg, or about 200 mg to about 400 mg, or about 400 mg to about 600 mg. In various further aspects, domatinostat is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for domatinostat is of from about 50 nM to about 100 nM, about 100 nM to about 200 nM, or about 200 nM to about 400 nM, or about 400 nM to about 600 nM. In various further aspects, the target whole blood concentration for domatinostat is about 600 nM to about 1 uM.

As would be understood by one of ordinary skill in the art, the whole blood concentration is determined by analyzing the pharmacokinetics of domatinostat (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of daporinad. The therapeutically effective amount of daporinad can be determined by methods known to one of skill in the art or as described elsewhere herein. Thus, in various aspects, the therapeutically effective amount of daporinad is administered every 4 weeks as a continuous intravenous infusion over 24 hours or more, including up to 96 hours at 0.018 mg/m²/h, 0.036 mg/m²/h, 0.072 mg/m²/h, 0.108 mg/m²/h, or 0.126 mg/m²/h. In other aspects, an oral or subcutaneous delivery formulation of daporinad can be used.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for daporinad is of from about 100 pM to about 1 nM, about 1 nM to about 2 nM, or about 2 nM to about 4 nM, or about 4 nM to about 10 nM, or about 10 nM to about 15 nM, or about 15 nM to about 30 nM In various further aspects, the target whole blood concentration for daporinad is about 100 pM to about 30 nM.

As would be understood by one of ordinary skill in the art, the whole blood concentration is determined by analyzing the pharmacokinetics of daporinad (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of alvespimycin. The therapeutically effective amount of alvespimycin can be determined by methods known to one of skill in the art or as described elsewhere herein. Thus, in various aspects, the therapeutically effective amount of alvespimycin is of from about 5 mg to about 10 mg, about 10 mg to about 20 mg, or about 20 mg to about 30 mg, or about 30 mg to about 40 mg, or about 40 mg to about 60 mg, or about 60 mg to about 80 mg In various further aspects, alvespimycin is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for alvespimycin is of from about 1 nM to about 5 nM, about 5 nM to about 10 nM, or about 10 nM to about 20 nM, or about 20 nM to about 50 nM, or about 50 nM to about 100 nM, or about 100 nM to about 200 nM, or about 200 nM to about 1 μM. In various further aspects, the target whole blood concentration for alvespimycin is about 10 nM to about 200 nM.

As would be understood by one of ordinary skill in the art, the whole blood concentration is determined by analyzing the pharmacokinetics of alvespimycin (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of navitoclax. The therapeutically effective amount of navitoclax can be determined by methods known to one of skill in the art or as described elsewhere herein. Thus, in various aspects, the therapeutically effective amount of navitoclax is of from about 1 mg to about 100 mg, about 100 mg to about 200 mg, or about 200 mg to about 400 mg, or about 400 mg to about 600 mg. In various further aspects, navitoclax is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for navitoclax is of from about 100 nM to about 200 nM, about 200 nM to about 500 nM, or about 500 nM to about 1,000 nM, or about 1,000 nM to about 2,000 nM. In various further aspects, the target whole blood concentration for navitoclax is about 500 nM to about 2 uM.

As would be understood by one of ordinary skill in the art, the whole blood concentration is determined by analyzing the pharmacokinetics of navitoclax (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of PD0325901. The therapeutically effective amount of PD0325901 can be determined by methods known to one of skill in the art or as described elsewhere herein. Thus, in various aspects, the therapeutically effective amount of PD0325901 is of from about 1 mg to about 100 mg, about 100 mg to about 200 mg, or about 200 mg to about 400 mg, or about 400 mg to about 600 mg. In various further aspects, PD0325901 is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for PD0325901 is of from about 1 nM to about 10 nM, about 10 nM to about 100 nM, or about 100 nM to about 500 nM, or about 500 nM to about 1000 nM. In various further aspects, the target whole blood concentration for PD0325901 is about 1 nM to about 128 nM.

As would be understood by one of ordinary skill in the art, the whole blood concentration is determined by analyzing the pharmacokinetics of PD0325901 (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of pimasertib. The therapeutically effective amount of pimasertib can be determined by methods known to one of skill in the art or as described elsewhere herein. Thus, in various aspects, the therapeutically effective amount of pimasertib is of from about 1 mg to about 100 mg, about 100 mg to about 200 mg, or about 200 mg to about 400 mg, or about 400 mg to about 600 mg. In various further aspects, pimasertib is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for pimasertib is of from about 100 nM to about 200 nM, about 200 nM to about 400 nM, or about 400 nM to about 800 nM, or about 800 nM to about 1600 nM. In various further aspects, the target whole blood concentration for pimasertib is about 100 nM to about 400 nM.

As would be understood by one of ordinary skill in the art, the whole blood concentration is determined by analyzing the pharmacokinetics of pimasertib (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of panobinostat. The therapeutically effective amount of panobinostat be determined by methods known to one of skill in the art or as described elsewhere herein. Thus, in various aspects, the therapeutically effective amount of panobinostat of from about 1 mg to about 100 mg, about 100 mg to about 200 mg, or about 200 mg to about 400 mg, or about 400 mg to about 600 mg. In various further aspects, panobinostat is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for panobinostat is of from about 40 nM to about 50 nM, about 50 nM to about 60 nM, or about 60 nM to about 70 nM, or about 70 nM to about 80 nM, or about 80 nM to about 1 μM. In various further aspects, the target whole blood concentration for panobinostat is about 40 nM to about 80 nM.

As would be understood by one of ordinary skill in the art, the whole blood concentration is determined by analyzing the pharmacokinetics of panobinostat (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of belinostat. The therapeutically effective amount of belinostat be determined by methods known to one of skill in the art or as described elsewhere herein. Thus, in various aspects, the therapeutically effective amount of belinostat of from about 1 mg to about 100 mg, about 100 mg to about 200 mg, or about 200 mg to about 400 mg, or about 400 mg to about 600 mg. In various further aspects, belinostat is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for belinostat is of from about 50 nM to about 100 nM, about 100 nM to about 200 nM, or about 200 nM to about 300 nM, or about 300 nM to about 1 μM. In various further aspects, the target whole blood concentration for belinostat is about 100 nM to about 300 nM.

As would be understood by one of ordinary skill in the art, the whole blood concentration is determined by analyzing the pharmacokinetics of belinostat (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of givinostat. The therapeutically effective amount of givinostat be determined by methods known to one of skill in the art or as described elsewhere herein. Thus, in various aspects, the therapeutically effective amount of givinostat of from about 1 mg to about 100 mg, about 100 mg to about 200 mg, or about 200 mg to about 400 mg, or about 400 mg to about 600 mg. In various further aspects, givinostat is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for givinostat is of from about 200 nM to about 300 nM, about 300 nM to about 400 nM, or about 400 nM to about 500 nM, or about 500 nM to about 1 μM. In various further aspects, the target whole blood concentration for givinostat is about 300 nM to about 500 nM.

As would be understood by one of ordinary skill in the art, the whole blood concentration is determined by analyzing the pharmacokinetics of givinostat (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of quisinostat. The therapeutically effective amount of quisinostat be determined by methods known to one of skill in the art or as described elsewhere herein. Thus, in various aspects, the therapeutically effective amount of quisinostat of from about 1 mg to about 100 mg, about 100 mg to about 200 mg, or about 200 mg to about 400 mg, or about 400 mg to about 600 mg. In various further aspects, quisinostat is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for quisinostat is of from about 100 pM to about 1 nM, about 1 nM to about 10 nM, or about 10 nM to about 20 nM, or about 20 nM to about 40 nM, or about 40 nM to about 80 nM, or about 80 nM to about 500 nM, or about 500 nM to about 1 μM. In various further aspects, the target whole blood concentration for quisinostat is about 10 nM to about 80 nM.

As would be understood by one of ordinary skill in the art, the whole blood concentration is determined by analyzing the pharmacokinetics of quisinostat (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

In various aspects, the method comprises administering a therapeutically effective amount of fimepinostat. The therapeutically effective amount of fimepinostat be determined by methods known to one of skill in the art or as described elsewhere herein. Thus, in various aspects, the therapeutically effective amount of fimepinostat of from about 1 mg to about 100 mg, about 100 mg to about 200 mg, or about 200 mg to about 400 mg, or about 400 mg to about 600 mg. In various further aspects, fimepinostat is administered at least one per day, at least twice per day, or at least three times per day.

In various aspects, the therapeutically effective amount is determined based on the target whole blood concentration. As would be understood by one of ordinary skill in the art, the target whole blood concentration will vary based on the drug selected. Thus, in various aspects, the target whole blood concentration for fimepinostat is of from about 100 pM to about 1 nM, about 1 nM to about 5 nM, or about 5 nM to about 10 nM, or about 10 nM to about 100 nM, or about 100 nM to about 500 nM, or about 500 nM to about 1 μM. In various further aspects, the target whole blood concentration for fimepinostat is about 5 nM to about 10 nM.

As would be understood by one of ordinary skill in the art, the whole blood concentration is determined by analyzing the pharmacokinetics of fimepinostat (and in the aspects below of each drug used) using a bioanalytical method such as but not limited to high-performance liquid chromatography (HPLC), liquid chromatography-tandem mass spectrometry (LC-MS/MS), conducted on blood samples collected at different time points from patients treated by the drug for varying periods of time ranging from one day to several weeks. If such analysis already exists in patients, it can be utilized to inform any future trials and treatment schedules. The pharmacokinetic analysis is used to determine the dosage and dosing schedules of drug and can be modified and/or selected to achieve the target range.

A therapeutically effective amount of a compound refers to an amount that is effective in controlling, reducing, or suppressing the growth or expression of microscopic metastatic tumors.

A therapeutically effective amount of the compound used in the treatment described herein can be readily determined by the attending diagnostician, as one of ordinary skill in the art, by the use of conventional techniques and by observing results obtained under analogous circumstances. In determining the therapeutically effective dose, a number of factors are considered by the attending diagnostician, including, but not limited to: the species of mammal; its size, age, and general health; the specific disease or condition involved; the degree of or involvement or the severity of the disease or condition; the response of the individual subject; the particular compound administered; the mode of administration; the bioavailability characteristic of the preparation administered; the dose regimen selected; the use of concomitant medication; and other relevant circumstances.

A therapeutically effective amount of a compound also refers to an amount of the compound which is effective in controlling, reducing or eliminating cancer or another condition described herein.

A therapeutically effective amount of the compositions will generally comprise sufficient active ingredient (e.g., a compound listed in Table 1 or Table 2 or any salt, ester, or derivative thereof) in the range from about 0.1 g/kg to about 100 mg/kg (weight of active ingredient/body weight of patient). Preferably, the composition will include at least 0.5 g/kg to 50 mg/kg, and more preferably at least 1 g/kg to 10 mg/kg of active ingredient.

Practice of the method comprises administering to a subject a therapeutically effective amount of the active ingredient, in any suitable systemic or local formulation, in an amount effective to deliver the dosages listed above. An effective, particularly preferred dosage of a compound (e.g., a compound listed in Tables 1 or 2 or any salt, ester, or derivative thereof)) for inhibiting suppressed cancer cells is 1 g/kg to 1 mg/kg of the active ingredient. The dosage can be administered on a one-time basis, or (for example) from one to five times per day or once or twice per week, or continuously via a venous drip, depending on the desired therapeutic effect.

As noted, amounts and modes of administration can be determined by one of ordinary skill in the art. One of ordinary skill in the art of preparing formulations can readily select the proper form and mode of administration depending upon the particular characteristics of the compound selected, the disease state to be treated, the stage of the disease, and other relevant circumstances using formulation technology known in the art, described, for example, in Remington's Pharmaceutical Sciences, latest edition, Mack Publishing Co.

Pharmaceutical compositions can be manufactured utilizing techniques known in the art. In some aspects, the therapeutically effective amount of the compound will be admixed with a pharmaceutically acceptable carrier.

The disclosed therapeutic agents or compositions may be administered by a variety of routes, for example, orally, intrarectally, or parenterally (i.e., subcutaneously, intravenously, intramuscularly, intraperitoneally, or intratracheally).

For oral and intrarectal administration, the therapeutic agents can be formulated into solid or liquid preparations such as capsules, suppositories, pills, tablets, lozenges, melts, liposomes, stealth-liposomes, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In another embodiment, the disclosed therapeutic agents can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations can be prepared by dissolving the active ingredient in an aqueous or non-aqueous pharmaceutically acceptable solvent which may also contain suspending agents, sweetening agents, flavoring agents, and preservative agents as are known in the art.

For parenteral administration, the therapeutic agents can be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, and buffers as are known in the art.

The disclosed therapeutic agents can also be administered topically. This can be accomplished by simply preparing a solution of the compound to be administered, preferably using a solvent known to promote transdermal absorption such as ethanol or dimethyl sulfoxide (DMSO) with or without other excipients. In some aspects, topical administration will be accomplished using a patch either of the reservoir and porous membrane type or of a solid matrix variety. Topical administration is also intended to refer to intrarectal administration which causes a topical effect on the intraluminal surface.

As noted above, the compositions disclosed herein can also include an appropriate carrier. For topical use, any of the conventional excipients may be added to formulate the active ingredients into a lotion, ointment, powder, cream, spray, or aerosol. For surgical implantation, the active ingredients may be combined with any of the well-known biodegradable and bioerodible carriers, such as polylactic acid and collagen formulations. Such materials may be in the form of solid implants, sutures, sponges, wound dressings, and the like. In any event, for local use of the materials, the active ingredients will usually be present in the carrier or excipient in a weight ratio of from about 1:1000 to 1:20,000, but are not limited to ratios within this range. Preparation of compositions for local use are detailed in Remington's Pharmaceutical Sciences, latest edition, (Mack Publishing).

Additional pharmaceutical methods can be employed to control the duration of action. Increased half-life and controlled release preparations may be achieved through the use of polymers to conjugate, complex with, or absorb the anti-cancer therapeutic agents described herein. The controlled delivery and/or increased half-life may be achieved by selecting appropriate macromolecules (for example, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide, and the appropriate concentration of macromolecules as well as the methods of incorporation in order to control release.

For example, the duration of action of the drugs by use of controlled release preparations may be accomplished by incorporation of the compound or its functional derivatives into particles of a polymeric material such as polyesters, polyamides, polyamino acids, hydrogels, poly(lactic acid), ethylene vinylacetate copolymers, copolymer micelles of, for example, PAM and poly(1-aspartamide).

It is also possible to entrap the therapeutic agents in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nanoparticles, and nanocapsules), or in macroemulsions. Such techniques are disclosed in the latest edition of Remington's Pharmaceutical Sciences.

U.S. Pat. No. 4,789,734 describes methods for encapsulating biochemicals in liposomes and is hereby expressly incorporated by reference herein. Essentially, the material is dissolved in an aqueous solution, the appropriate phospholipids and lipids added, along with surfactants if required, and the material dialyzed or sonicated, as necessary. A review of known methods is by G. Gregoriadis, Chapter 14. “Liposomes,” Drug Carriers in Biology and Medicine, pp. 287-341 (Academic Press, 1979). Microspheres formed of polymers or proteins are well known to those skilled in the art and can be tailored for passage through the gastrointestinal tract directly into the blood stream. Alternatively, the agents can be incorporated and the microspheres, or composite of microspheres, implanted for slow release over a period of time, ranging from days to months. See, for example, U.S. Pat. Nos. 4,906,474; 4,925,673; and 3,625,214 which are incorporated by reference herein.

In some aspects, when the composition is to be used as an injectable material, the composition can be formulated into a conventional injectable carrier. Suitable carriers include biocompatible and pharmaceutically acceptable phosphate buffered saline solutions, which are preferably isotonic.

For reconstitution of a lyophilized product, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field.

The pharmaceutical composition disclosed herein can also be in the form of an aqueous solution containing many of the same substances as described above for the reconstitution of a lyophilized product.

In some aspects, the therapeutic agents can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As mentioned above, the disclosed therapeutic agents may be incorporated into pharmaceutical preparations which may be used for therapeutic purposes. However, the term “pharmaceutical preparation” is intended in a broader sense herein to include preparations containing a compound in used not only for therapeutic purposes but also for reagent, screening, or diagnostic purposes as known in the art, or for tissue culture. The pharmaceutical preparation intended for therapeutic use should contain a “pharmaceutically acceptable” or “therapeutically effective amount” of a compound, i.e., that amount necessary for preventative or curative health measures. If the pharmaceutical preparation is to be employed as a reagent or diagnostic, then it should contain reagent or diagnostic amounts of a compound.

The methods disclosed herein can also be used to analyze the mode of action of compositions described herein or other compositions such as approved drugs in treatment of cancer in a human subject. For example, establishing if such a composition acts by eliminating dormant cancer cells, or by keeping the in a dormant state, or both. The methods can entail obtaining a biopsy of a tumor from a metastatic site such as the lungs, liver or bone, or from the bone marrow before treatment is initiated and comparing the second biopsy from one of the sites above following treatment and examining pathology, genetic or by using any other suitable methods determining if initial cancer cells have exited dormancy or have been eradicated by the treatment.

The methods disclosed herein can also be used to analyze the mode of action of compositions described herein or other compositions such as approved drugs in treatment of cancer in a human subject. For example, establishing if such a composition acts by eliminating microscopic metastatic tumors, or by preventing the microscopic metastatic tumor to escape a dormant, no growth or slow growth state, or a combination of eliminating them and keeping them from exciting dormant, no growth or slow growth state. The methods can entail obtaining a biopsy of a tumor from a metastatic site such as the lungs, liver or bone, or from the bone marrow before treatment is initiated and comparing the second biopsy from one of the sites above following treatment and examining pathology, genetic or by using any other suitable methods determining if initial cancer cells have exited dormancy or have been eradicated by the treatment.

The methods disclosed herein can also be used to recommend further treatment based on the results of such analysis including decisions on continued dosing and increased dosing of a dormastatic (prevents outbreak or rapid growth of dormant cancer cells) or dormacidal (kills dormant cancer cells) drug, or combination with other compositions or treatments. Based on the manner in which the compound acts, further treatment guidelines can be suggested. For example, if following treatment with a compound dormant cancer cells are illuminated the dosage or duration of treatment may be reduced. As yet another example is following treatment dormant cells remain but remain in a dormant state over time, the dosage and duration of treatment may be increased.

The methods disclosed herein can also be used to recommend further treatment based on the results of such analysis including decisions on continued dosing and increased dosing of a drug that prevents outbreak or rapid growth of microscopic metastatic tumor cancer cells or kills microscopic metastatic tumors, or combination with other compositions or treatments. Based on the manner in which the compound acts, further treatment guidelines can be suggested. For example, if following treatment with a compound microscopic metastatic tumor are eliminated the dosage or duration of treatment may be reduced. As yet another example is following treatment microscopic metastatic tumors remain in a dormant, or no growth state over time, the dosage and duration of treatment may be increased.

### G. EXAMPLES

The following examples further illustrate this disclosure. The scope of the disclosure and claims is not limited by the scope of the following examples.

### 1. BME Assay

3D basement membrane extract (BME) assay was developed at the NCI Barkan (D. Barkan et al. 2008; Dalit Barkan and Green 2011; Hen and Barkan 2020). BME is purified from Engelbreth-Holm-Swarm (EHS) tumor, primarily made up of laminin, collagen IV, entactin, and heparin sulfate proteoglycans (Cultrex Reduced Growth Factor Basement Membrane Extract, PathClear n.d.), which allows it to more accurately reflect the 3D extracellular environment in the body.

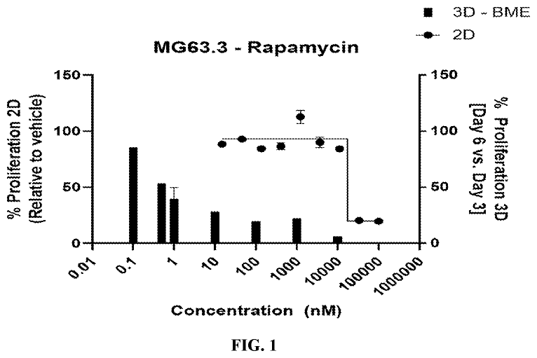

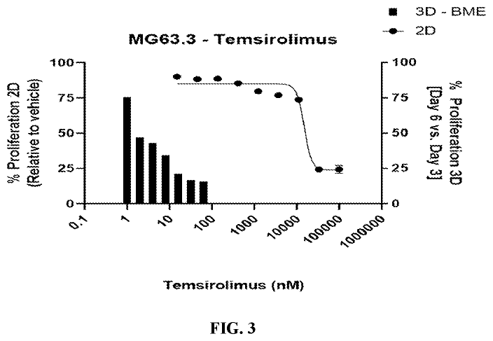

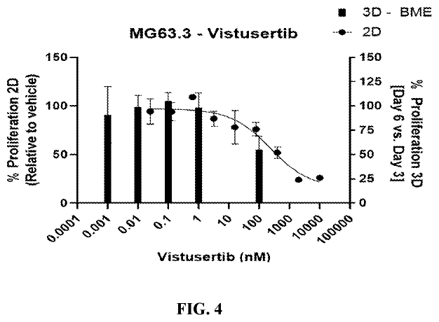

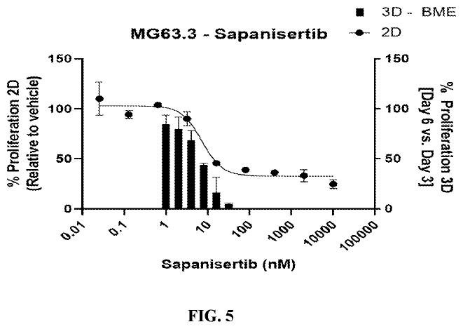

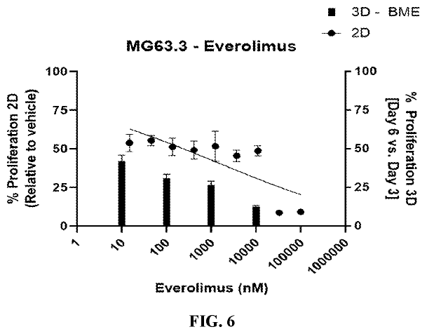

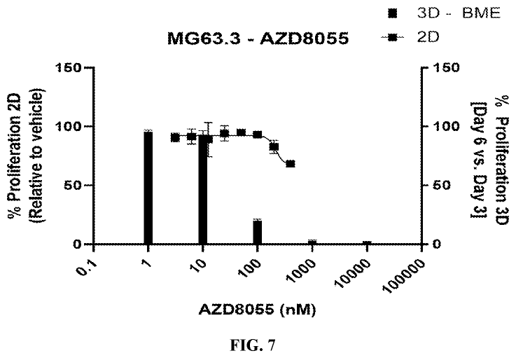

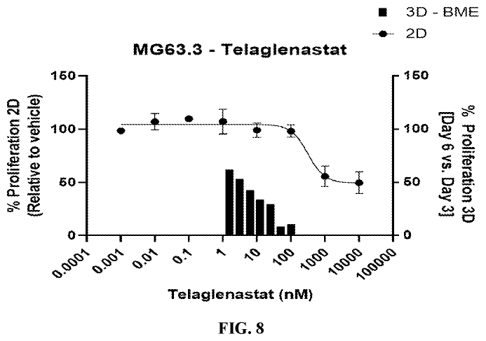

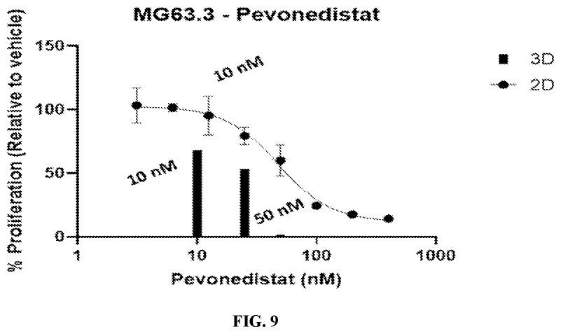

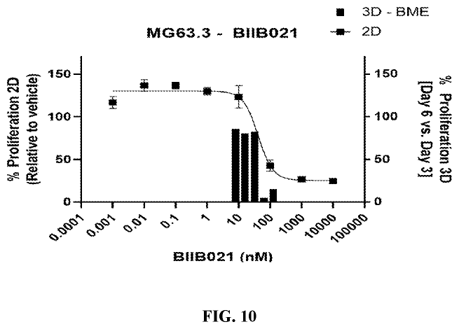

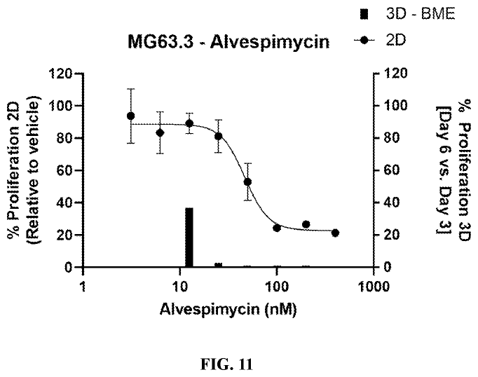

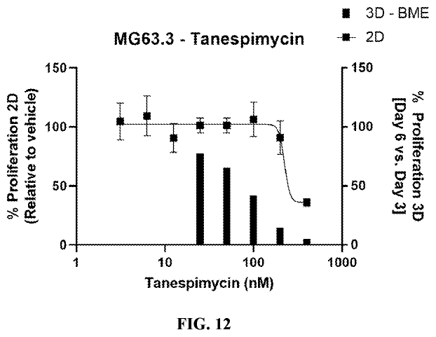

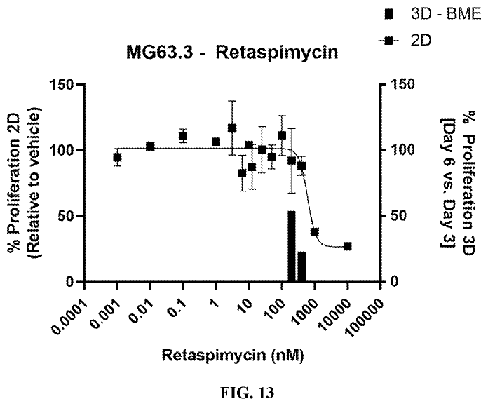

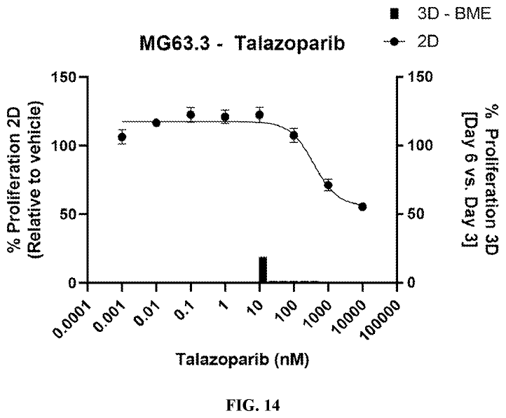

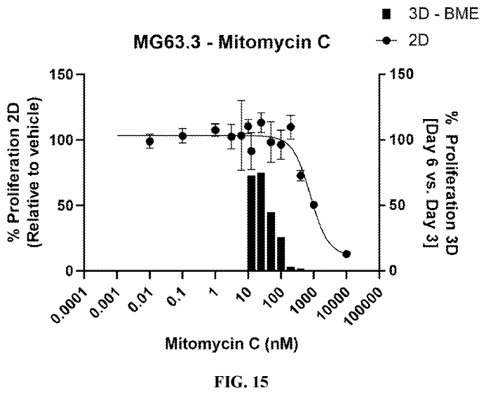

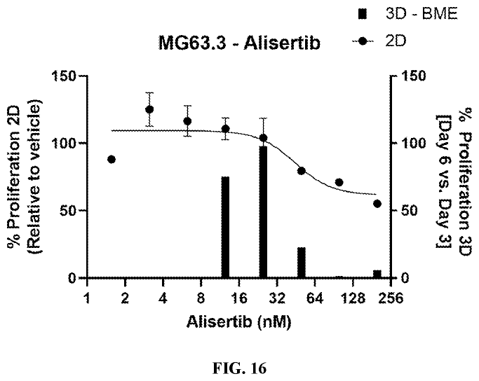

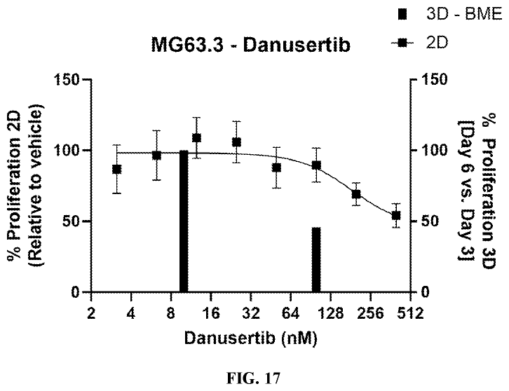

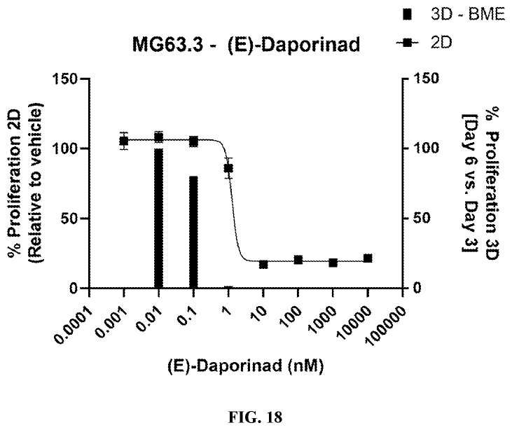

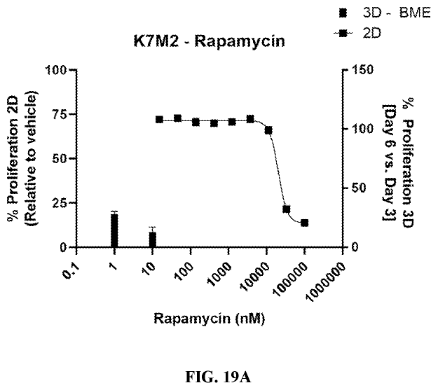

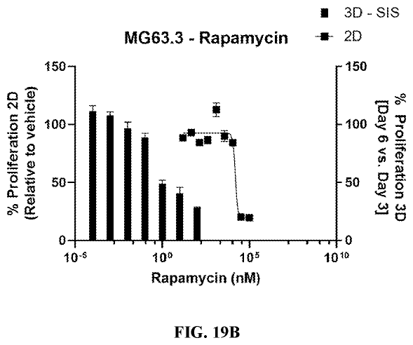

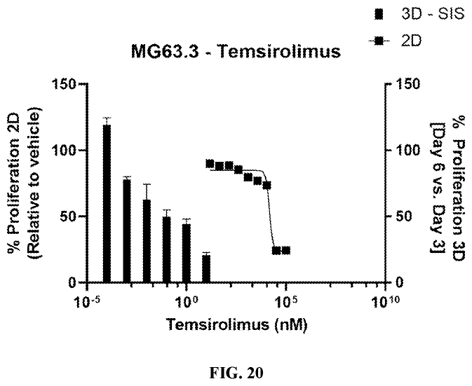

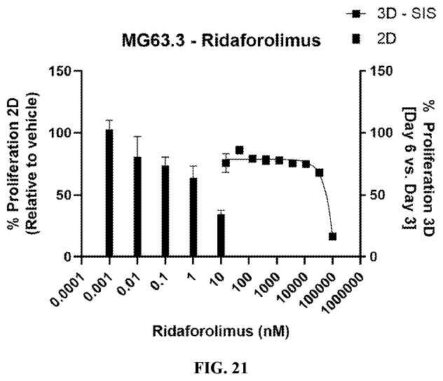

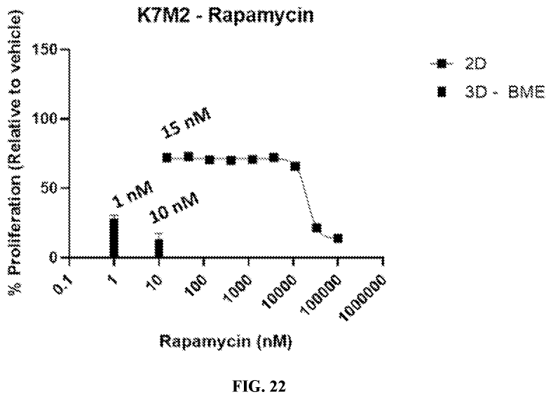

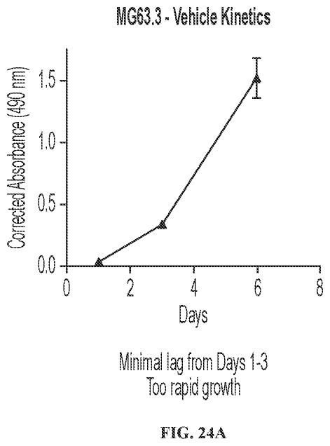

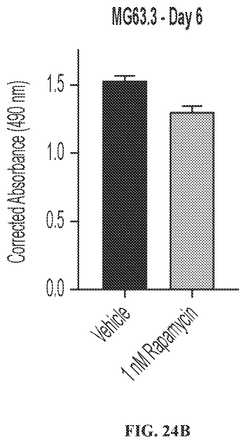

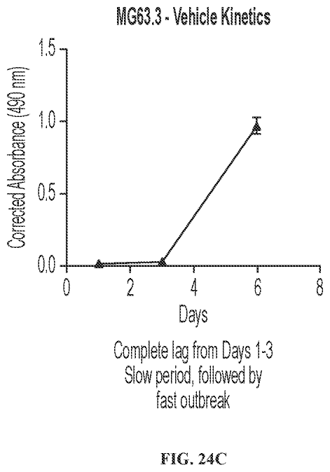

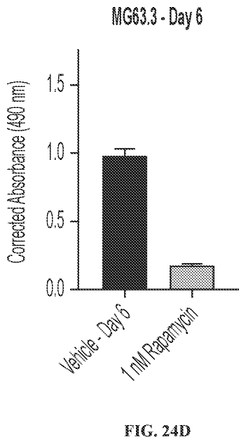

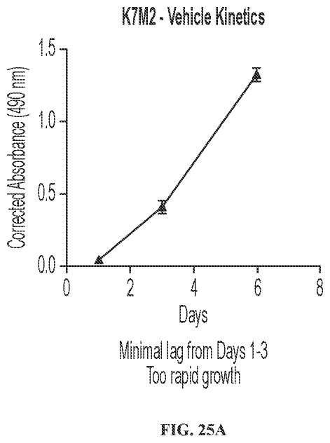

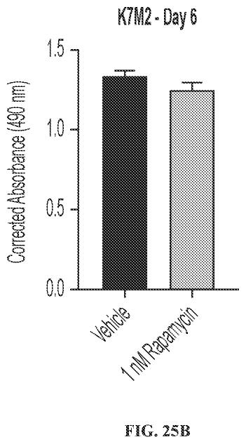

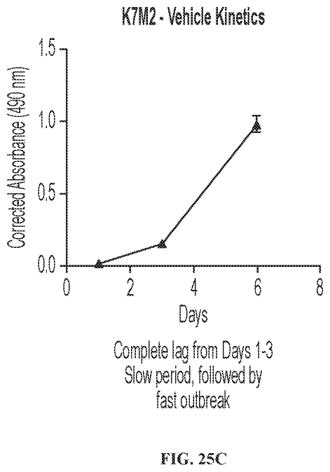

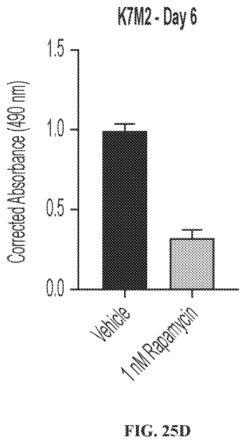

Since metastatic cancer cells grown in 3D assays rely on ME, screens can identify drugs that selectively target it. Therefore, a potency criterium is applied, where hits must not only exhibit statistically significant inhibition of highly metastatic lines on BME, but do so at a doses that are lower than the 2D IC₅₀ (as evaluated by MTT), and with a desired target of ≥100-fold lower dose. This criterium inherent to the screen also results in a strong possibility that hits do not induce significant cytotoxicity that may lead to unwanted side-effects. This toxicity is of particular concern when developing drugs that will selectively prevent cancer recurrence, as these drugs may need to be taken safely for long periods of time. It is anticipated that drugs will be administered safely for long terms, in low doses to patients during remission. Initially >150 selected therapeutic agents were tested on BME based on the likelihood for either a positive effect or a many that were rationally selected based on metastasis-focused mechanisms identified in OS negative effect (as negative controls). Several doses where used (0.1, 1 & 10 μM) and multiple hits were identified that led to either significant or near-complete inhibitory activity on highly metastatic OS cell lines in 3D. These therapeutic agents were also tested in dose response (as broad as 1 pM-100 μM) for 2D cytotoxicity (MTT) and the IC₅₀ was calculated. Where required, the hits from the initial screen were also further characterized at additional doses <100 nM in order to further determine the relative selectivity compared to the 2D IC50. Compounds that are active selectively on 3D are deemed candidate ME targeting therapeutic agents. Representative BME data in both the human and murine OS models show that strongly active therapeutic agents (maintain a near identical rate of growth similar to that observed from the non-proliferating period from Days 1-3 (FIG. 24 (MG63.3) and FIG. 25 (K7M2)), while moderately active therapeutic agents provide significant decreases of Day 6 growth relative to vehicle). In addition to their activity on BME, all therapeutic agents that are active (FIG. 1-22) are more active on BME than in 2D. For example, rapamycin a significantly inhibited ME in this assay at 1 nM (>10,000-fold lower than the 2D IC₅₀), indicating high selectivity against ME.

### 2. SISGel

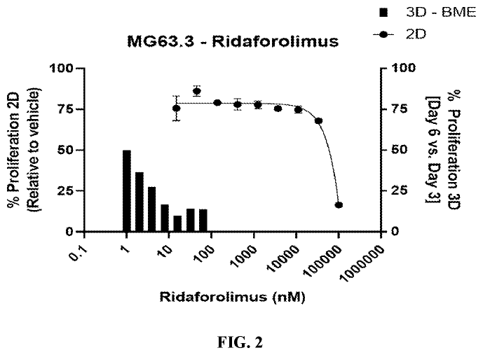

SISGel is comprised of porcine small intestine submucosa gel that was originally developed at Oklahoma State U., by R. Hurst (CSO) and M. Ihnat (Consultant) (Bailey-Downs et al. 2014; Hurst et al. 2013, 2015, 2016). Unlike BME which is derived from a tumor-based matrix, SISGel is derived from healthy stroma and provides a suppressive “normal” ECM. When cultured under conditions that promote ME in this system (including lower cell density), most cancer cell lines exhibit a suppressed phenotype. Conditions have been identified that promoted suppressed ME-type profile (FIG. 2A). Compounds shown to be active in BME (e.g. VUJ-0101) led to most significant decreases at Day 6 in the most stringent ME promoting conditions. In addition to SISGel's complementarity to the BME assay, it can also be conducted in a 96-well format, enabling a high-throughput system to model suppressed/dormant cancer cells to further validate the screening results in BME.

In addition to SISGel can also enable a high-throughput system to model suppressed microscopic metastatic tumor cancer cells to further validate the screening results in BME.

### 3. PuMA

PuMA (ex vivo Pulmonary Metastasis Assay) developed at the NCI by Chand Khanna and team, is a higher-throughput ex vivo alternative to classical in vivo metastasis assays (Mendoza et al. 2010; Morrow et al. 2016; Ren et al. 2015). Tumor cells are injected into the tail vein of the recipient mouse and after 15-30 mins the animal is euthanized, the lungs removed and filled with a mixture of agar and culture medium through the trachea to prevent them from collapsing so that the alveolar space can maintain its 3D structure. Dissected lungs are then sectioned and grown on sponges. Lung slices containing the seeded single cells are treated with various compounds and the impact of treatments on the ME phenotype is assessed over time using fluorescence microscopy. Drug treatment is initiated within 24 h and carried out for all or part of the up to 21-day duration. OS cells with low metastatic potential do not grow as metastatic foci despite their presence in lung slices on day 1. Highly metastatic OS cells with ME capacity eventually grow and form metastatic foci, which can be monitored over time and quantitated though fluorescence imaging. Similar to the progression observed in human cancers, tumor cells start as single cells and exhibit no observable growth for 10-12 days, followed by outbreak. Compounds that are able to inhibit ME lead to a significant reduction in fluorescent area as the drug either kills the initially seeded single cells or prevents their ability to outgrown after the initial lag in proliferation. The biological validity of this assay was confirmed by its prediction of the in vivo behavior of a variety of high- and low-metastatic human and mouse cancer cell lines by interrogating the interaction between the lung microenvironment and the metastatic cells.

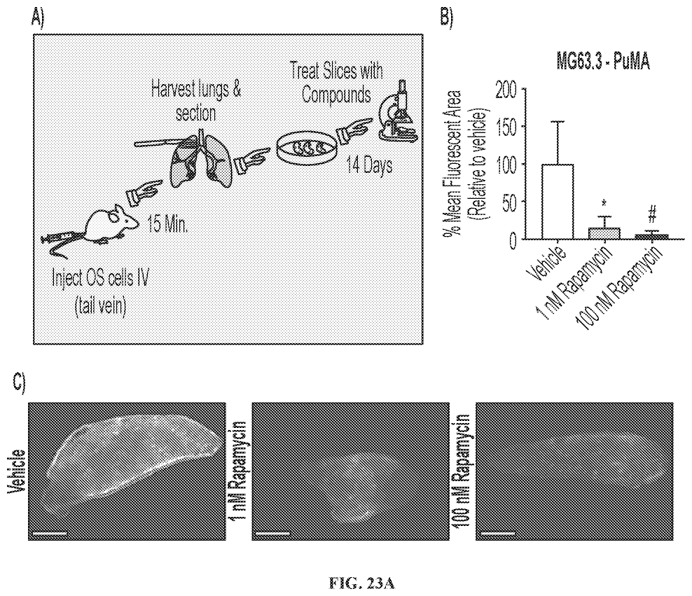

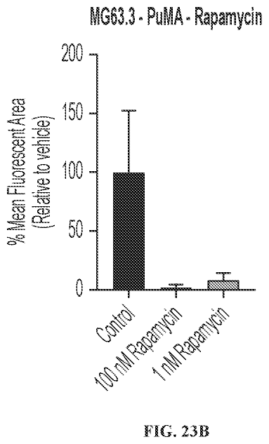

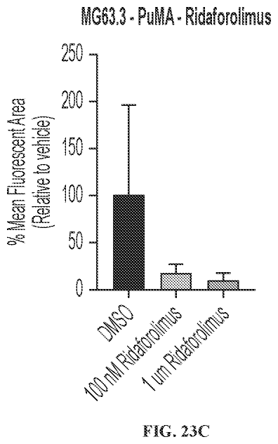

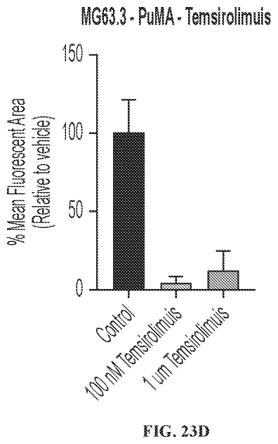

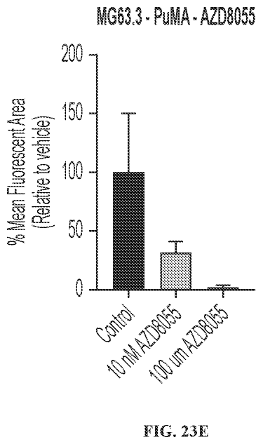

The mTOR inhibitors that were active on BME exhibited near complete inhibition of ME in PuMA at concentrations of 1 nM and higher ME (FIG. 23), with rapamycin exhibiting high activity at 1 nM. These data show the high potency observed with these compounds in another model of ME, as the activity in PuMA occurs at doses orders of magnitude lower than the 2D IC50. Representative images show that on Day 14, lung slices either treated with inactive compounds or vehicle have strong, diffuse fluorescent signal, indicating outbreak from their initial single-cell clusters (FIG. 23). Active compounds inhibit ME leading to significantly less survival and/or outbreak of these highly metastatic cells, represented by lower fluorescent area. These compounds exhibited a similar effect on MG63.3 cells in both BME & PuMA, supporting their activity against ME and dormant cancer cells as well as the complementarity of these assays.

The data generated herein also support the activity of mTOR inhibitors against microscopic metastatic tumors.

### 4. Evaluation of Therapeutic Agents

### A. Rapamycin

Highly metastatic OS MG63.3 (human) treated with rapamycin (100 pM-10 μM) lead to appreciable inhibition of 3D growth at doses as low as 500 pM (replicates=4; #P<0.005 for all doses ≥500 pM, all comparisons made between treatment and vehicle control). The effect of rapamycin on 2D growth (15 nM-100 μM) over 72 h was measure by MTT (replicates=3), with rapamycin only exhibiting cytotoxicity at doses ≥33 μM. Rapamycin treatment exhibited much stronger activity on 3D BME at significantly lower doses.

Highly metastatic cell line MG63.3 (human OS) can be grown in BME in conditions with certain numbers of cells and other parameters and either show a lag in proliferation for about 3 days, followed by increased proliferation (outbreak) by Day 6 or a lack of, or less pronounced lag. Rapamycin (1 nM) is used to assess if the cells are exhibiting the correct ME phenotype for activity of ME disabling drugs. In this example, 1 nM is marginally effective (FIG. 24A and FIG. 24B), meaning that in this experiment ME was not adequately achieved and this experiment cannot qualify for screening or testing for drugs that disable ME. In contrast, 1 nM is highly effective (FIG. 24C and FIG. 24D), meaning that in this experiment ME was achieved and this experiment can qualify for screening or testing for drugs that disable ME.

Highly metastatic OS line K7M2 (murine) can be grown in BME in conditions with certain numbers of cells and other parameters and either show a lag in proliferation for ˜3 days, followed by increased proliferation (outbreak) by Day 6 or a lack of, or less pronounced lag. Rapamycin (1 nM) is used to assess if the cells are exhibiting the correct ME phenotype for activity of ME disabling drugs. In this example, 1 nM is not effective (FIG. 25A and FIG. 25B), meaning that in this experiment ME was not achieved and this experiment cannot qualify for screening or testing for drugs that disable ME. In contrast, in this experiment, 1 nM is effective (FIG. 25C and FIG. 25D), meaning that in this experiment ME was achieved and this experiment can qualify for screening or testing for drugs that disable ME.

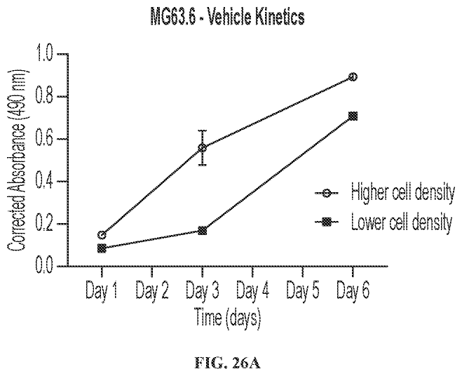

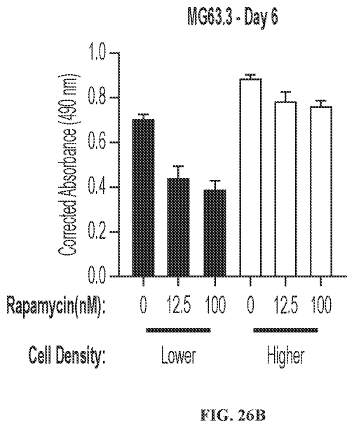

Highly metastatic cell line MG63.3 (human OS) can be grown in SIS gel in conditions with certain numbers of cells and other parameters and either show a lag in proliferation for ˜3 days, followed by increased proliferation (outbreak) by Day 6 or a lack of, or less pronounced lag. Rapamycin (1 nM) is used to assess if the cells are exhibiting the correct ME phenotype for activity of ME disabling drugs. In this example, 1 nM is marginally effective (FIG. 26A), meaning that in this experiment ME was not adequately achieved and this experiment cannot qualify for screening or testing for drugs that disable ME. In contrast, in this experiment, 1 nM is highly effective (FIG. 26B), meaning that in this experiment ME was achieved and this experiment can qualify for screening or testing for drugs that disable ME.

b. HDAC Inhibitors

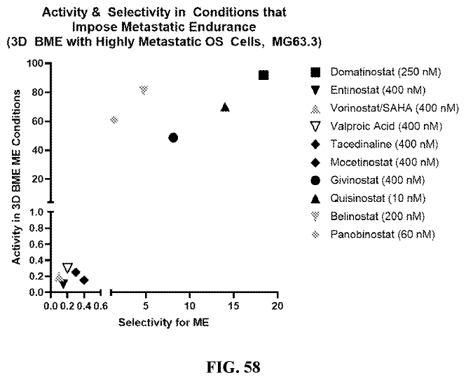

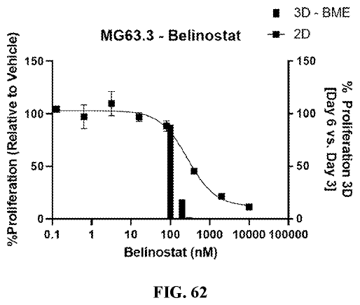

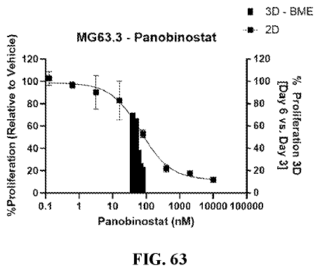

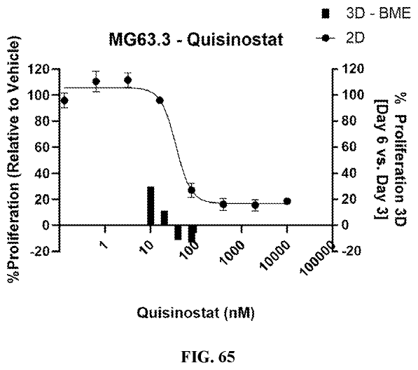

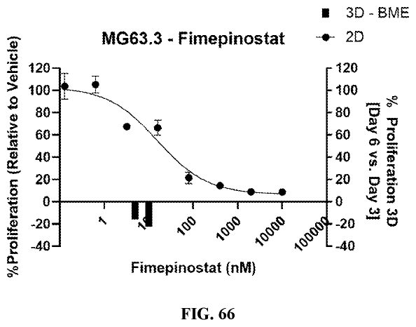

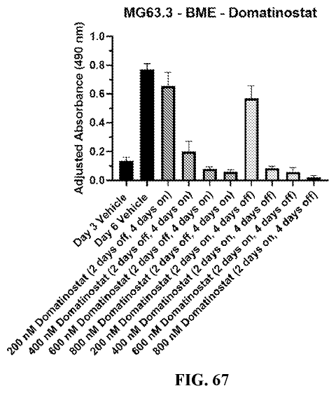

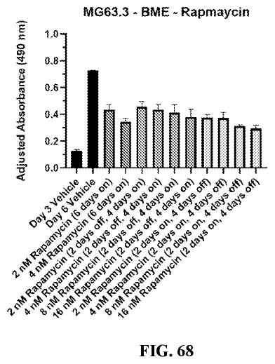

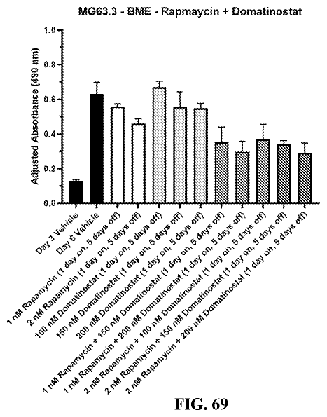

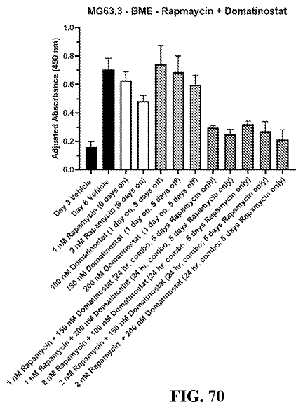

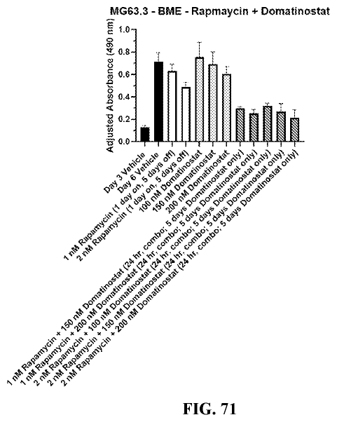

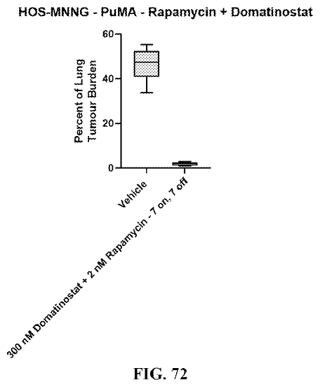

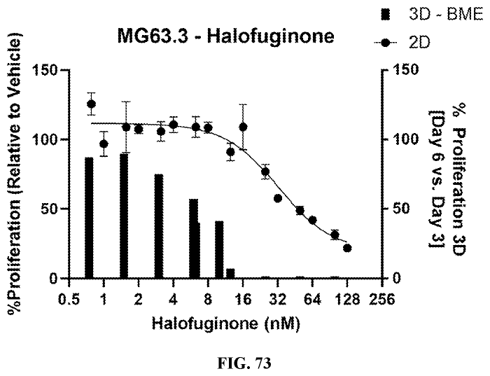

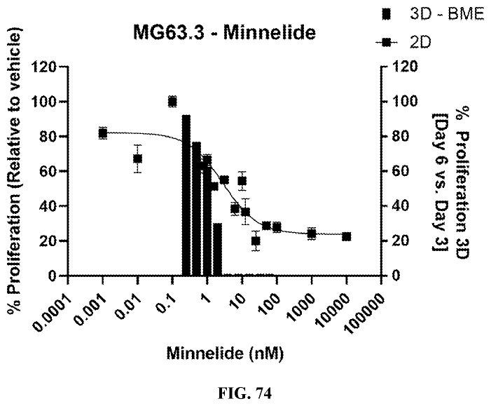

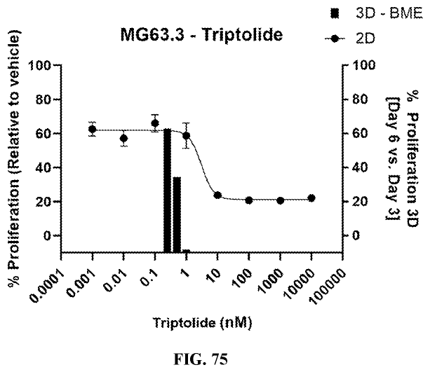

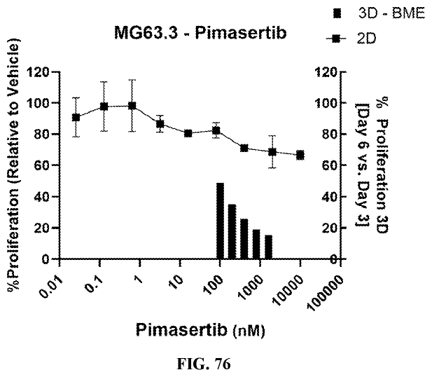

Highly metastatic human OS cell line MG63.3, when treated with domatinostat, shows the profile of a compound that is more active in 3D than in 2D. Compounds that are highly active and more selective to cells with an ME phenotype can be used to prioritize compounds for further study. Several HDAC inhibitors exhibited 3D activity and selectivity for ME of a screen of 10 HDAC inhibitors of varying selectivity (Class I, Class II, pan-HDAC) as shown in FIG. 58. Notably, these included the pan-HDAC inhibitors belinostat (FIG. 62) and panobinostat (FIG. 63) and the compound fimepinostat (FIG. 65).

c. Class I-Selective HDAC Inhibitors

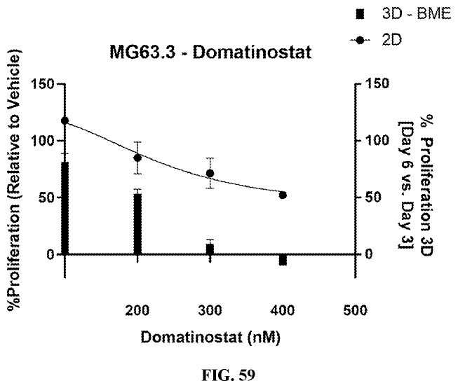

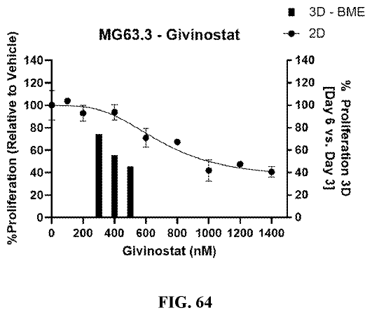

Highly metastatic human OS cell line MG63.3, when treated with class I-selective HDAC inhibitors domatinostat, givinostat and quisinostat, show the profile of a compound that is more active in 3D than in 2D. Compounds that are highly active and more selective to cells with an ME phenotype can be used to prioritize compounds for further study. In this example, at 100-200 nM domatinostat, there was significant growth inhibition (20-50%) in the 3D BME assay, while there was no statistically significant inhibition in 2D at these doses (FIG. 59). Doses of domatinostat up to 400 nM inhibits MG63.3 cells in the 3D BME assay to a much greater extent than the effect of domatinostat on 2D growth at these same doses. In addition, with 400 nM givinostat and 10 nM quisinostat, there was significant 3D growth inhibition and selectivity for ME in the 3D BME assay (see FIG. 58, FIG. 64, and FIG. 65). The Class I-selective HDAC inhibitors domatinostat, givinostat, and quisinostat all exhibited the highest 3D activity and selectivity for ME of a screen of 10 HDAC inhibitors of varying selectivity (Class I, Class II, pan-HDAC).

d. Navitoclax

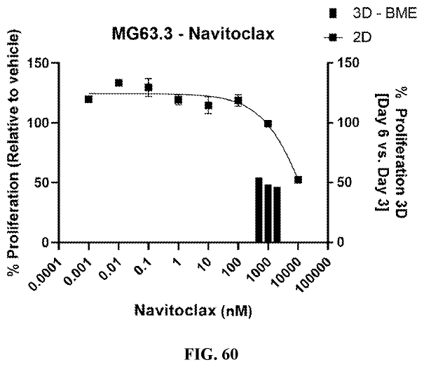

Highly metastatic human OS cell line MG63.3, when treated with navitoclax, shows the profile of a compound that is more active in 3D than in 2D. Compounds that are highly active and more selective to cells with an ME phenotype can be used to prioritize compounds for further study. In this example, at 500-2000 nM navitoclax, there was significant growth inhibition (50-60%) in the 3D BME assay, while there was little to no inhibition (0-25%) in 2D at these dose (FIG. 60). Doses of navitoclax up to 400 nM inhibits MG63.3 cells in the 3D BME assay to a much greater extent than the effect of navitoclax on 2D growth at these same doses.

e. PD0325901

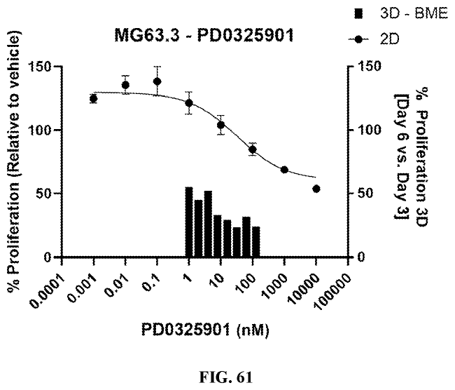

Highly metastatic human OS cell line MG63.3, when treated with PD0325901, shows the profile of a compound that is more active in 3D than in 2D. Compounds that are highly active and more selective to cells with an ME phenotype can be used to prioritize compounds for further study. In this example, at 1-128 nM PD0325901, there was significant growth inhibition (50-75%) in the 3D BME assay, while there was no statistically significant inhibition in 2D at this dose (FIG. 61). Doses of PD0325901 up to 400 nM inhibits MG63.3 cells in the 3D BME assay to a much greater extent than the effect of PD0325901 on 2D growth at these same doses.

f. Telaglenastat

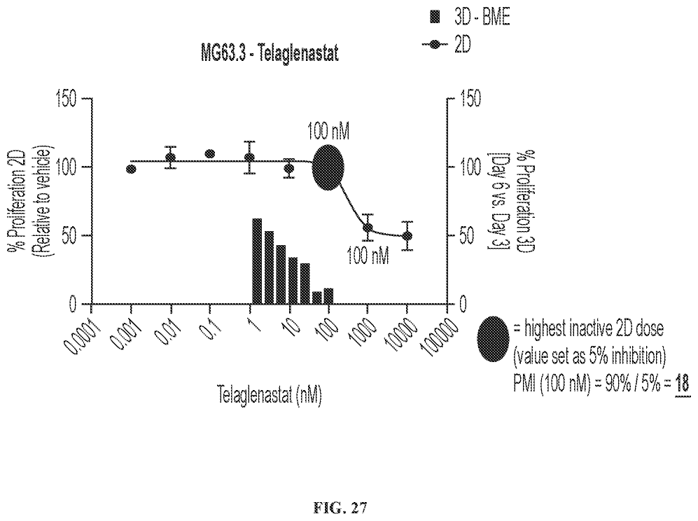

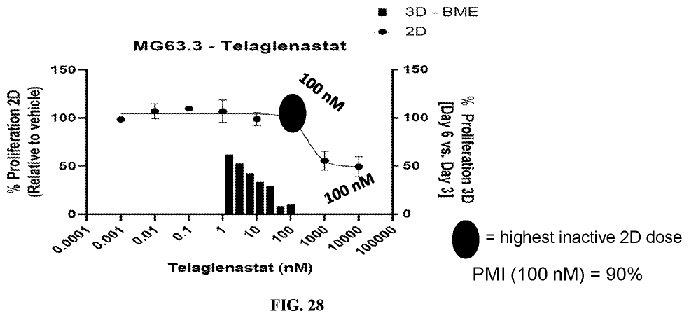

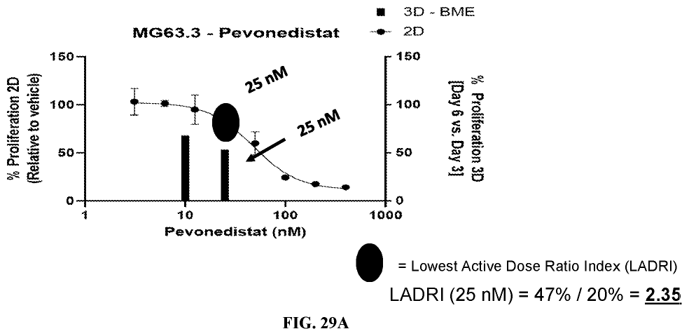

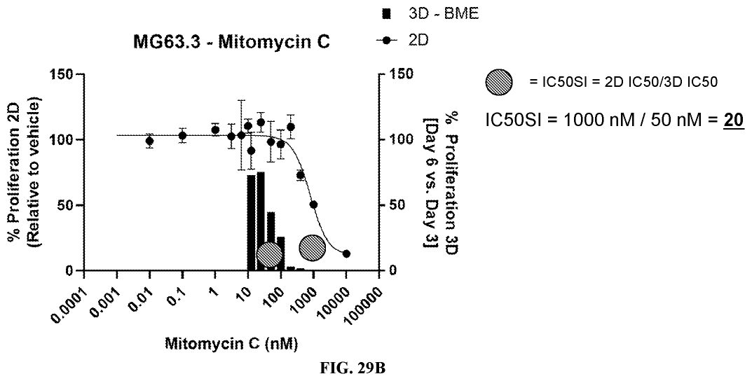

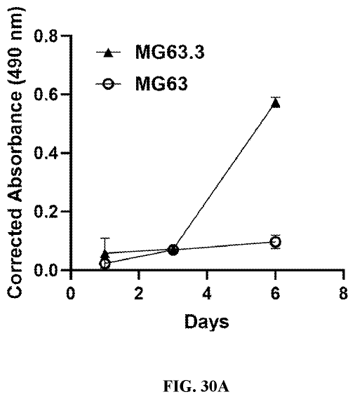

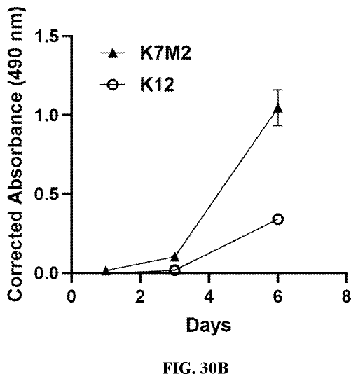

Highly metastatic human OS cell line MG63.3, when treated with telaglenastat, shows the profile of a compound that is more active in 3D than in 2D. Compounds that are highly active and more selective to cells with an ME phenotype can be used to prioritize compounds for further study. In this example, at 100 nM telaglenastat, there was 10% growth in the 3D BME assay, while there was no statistically significant inhibition in 2D at this dose (FIG. 27). This dose was chosen as it was the highest dose of telaglenastat that did not lead to significant 2D inhibition.

g. Pevonedistat

Highly metastatic human OS cell line MG63.3, when treated with pevonedistat, shows the profile of a compound that is more active in 3D than in 2D, exhibiting selectivity using the lowest active dose ratio index (LARDI). Compounds that have a high LARDI are more efficacious at a similar dose against cells with an ME phenotype and LARDI can be used to prioritize compounds for further study. In this example, 25 nM pevonedistat led to 47% inhibition in 3D BME, and 20% inhibition in 2D. This was the lowest dose of pevonedistat that led to significant inhibition in 2D. Thus, the LARDI of 25 nM pevonedistat is 47%/20%=2.35.

h. Mitomycin C

Highly metastatic human OS cell line MG63.3, when treated with mitomycin C, shows the profile of a compound that is more active in 3D than in 2D, exhibiting selectivity using the IC₅₀ selectivity index (IC₅₀SI). Compounds that have a high IC₅₀SI are more potent and selective against cells with an ME phenotype and IC₅₀SI can be used to prioritize compounds for further study. In this example, the 2D IC₅₀ was determined to be 1000 nM, while the 3D IC₅₀ was 50 nM. Thus, the IC₅₀SI of mitomycin C is 20.

### 5. Selection of Dosing Regimens

In one aspect, disclosed are methods of selecting and/or optimizing dosing regimen. Target whole blood sirolimus (API is Rapamycin) concentration ranges from 5 to 15 ng/mL. When given alone (at 2 mg/day), the trough concentration range across months 4 through 12 was 14-22 ng/mL (mean=19) and in year 3 the range was 11-22 ng/mL (mean=16). Following cyclosporine withdrawal in transplant patients at low-to moderate-immunologic risk, the target sirolimus trough concentrations should be 16 to 24 ng/mL for the first year following transplantation. Thereafter, the target sirolimus concentrations should be 12 to 20 ng/mL. Sirolimus pharmacokinetics displays wide inter- and intra-patient variability.

Based on methods of the invention, it is predicted that target whole blood sirolimus (API is rapamycin) concentration ranges from 1 to 5 ng/mL and higher are effective against metastatic endurance of metastatic cancer cells. Therefore when given alone, or in combination safe and efficacious levels are up to 10 mg, including 0.5, 0.75, 1, 1.25, 1.5, 1.75, 2, 2.5, 3, 3.5, 4, 4.5, 5, 6, 7, 8, 9, or 10 mg, given once a day or multiple times a day).

In one aspect, disclosed are methods of selecting and/or optimizing dosing regimen for target whole blood concentration ranges, including but not limited to the following drugs:
Domatinostat concentration ranges from 100 to 1000 nM and higher are effective against metastatic endurance of metastatic cancer cells. Therefore, the target domatinostat concentrations should be 100 to 1,000 nM and higher.
Daporinad concentration ranges from 0.5 nM to 10 nM and higher are effective against metastatic endurance of metastatic cancer cells. Therefore, the target daporinad concentrations should be 0.5 nM to 10 nM and higher.
Alvespimycin concentration ranges from 10 to 200 nM and higher are effective against metastatic endurance of metastatic cancer cells. Therefore, the target alvespimycin concentrations should be 10 to 200 nM and higher.
Navitoclax concentration ranges from 500 to 2,000 nM and higher are effective against metastatic endurance of metastatic cancer cells. Therefore, the target navitoclax concentrations should be 500 to 2,000 nM and higher.
PD0325901 concentration ranges from 1 to 1,000 nM and higher are effective against metastatic endurance of metastatic cancer cells. Therefore, the target PD0325901 concentrations should be 1 to 1,000 nM and higher.
Pimasertib concentration ranges from 100 to 1,600 nM and higher are effective against metastatic endurance of metastatic cancer cells. Therefore, the target pimasertib concentrations should be 100 to 1,600 nM and higher.
Panobinostat concentration ranges from 500 to 400 nM and higher are effective against metastatic endurance of metastatic cancer cells. Therefore, the target panobinostat concentrations should be 500 to 400 nM and higher.
Belinostat concentration ranges from 160 to 400 nM and higher are effective against metastatic endurance of metastatic cancer cells. Therefore, the target belinostat concentrations should be 160 to 400 nM and higher.
Givinostat concentration ranges from 400 to 800 nM and higher are effective against metastatic endurance of metastatic cancer cells. Therefore, the target givinostat concentrations should be 400 to 800 nM and higher.
Quisinostat concentration ranges from 10 to 80 nM and higher are effective against metastatic endurance of metastatic cancer cells. Therefore, the target quisinostat concentrations should be 10 to 80 nM and higher.
Fimepinostat concentration ranges from 5 to 10 nM, and 10 nM to 10,000 nM and higher are effective against metastatic endurance of metastatic cancer cells. Therefore, the target fimepinostat concentrations should be 5 to 10 nM, and 10 nM to 10,000 nM and higher.

In one aspect, disclosed are methods of selecting and/or optimizing dosing regimen, including but not limited to the following drugs and dosing:
Rapamycin at 0.5, 1, 1.5, 2, 2.5, 3, 3.5, 4, 4.5, 5, 6, 7, 8, 9, or 10 mg, given once or twice a day.
Daporinad administered every 4 weeks as a continuous intravenous infusion over 24 hours or more, including up to 96 hours at 0.018 mg/m²/h, 0.036 mg/m²/h, 0.072 mg/m²/h, 0.108 mg/m²/h, or 0.126 mg/m²/h.
Domatinostat at 100, 150, 200, 250, 300, 350, 400, 450, or 500 mg, given once or twice a day.
Alvespimycin at 10, 15, 20, 25, 30, 35 or 40 mg, given once or twice a day given once or twice a day, or every 2 days or 3 days.

In one aspect disclosed are methods for treating a cancer patient in need, including by targeting minimal residual disease or preventing recurrence or targeting metastatic endurance, microscopic metastatic tumors or dormant cancer cells of using two or more drugs in combination. Such drugs and dosing combinations include but are not limited to the following, when given to a patient orally as separate pills, as a fixed dose pill, or parentally, or in combination of an oral, or parenteral administration, one or more times a day, less than once a day, together or in sequence:
Rapamycin and domatinostat: Such combination may be of rapamycin at 0.5, 1, 1.5, 2, 2.5, 3, 3.5, 4, 4.5, 5, 6, 7, 8, 9 or 10 mg, given once or twice a day and of domatinostat at 100, 150, 200, 250, 300, 350, 400, 450, or 500 mg, given once or twice a day.
Rapamycin and daporinad: Such combination may be of rapamycin at 0.5, 1, 1.5, 2, 2.5, 3, 3.5, 4, 4.5, 5, 6, 7, 8, 9 or 10 mg, given once or twice a day and of daporinad daporinad given intravenously at 0.018 mg/m²/h, 0.036 mg/m²/h, 0.072 mg/m²/h, 0.108 mg/m²/h, or 0.126 mg/m²/h.
Domatinostat+daporinad. Such combination may be of domatinostat at 100, 150, 200, 250, 300, 350, 400, 450, or 500 mg, given once or twice a day and of daporinad given intravenously at 0.018 mg/m²/h, 0.036 mg/m²/h, 0.072 mg/m²/h, 0.108 mg/m²/h, or 0.126 mg/m²/h.
Rapamcyin+alvespimycin. Such combination may be of rapamycin at 0.5, 1, 1.5, 2, 2.5, 3, 3.5, 4, 4.5, 5, 6, 7, 8, 9, or 10 mg, given once or twice a day and of alvespimycin at 10, 15, 20, 25, 30, 35 or 40 mg, given once or twice a day, or every 2 days or 3 days.
Domatinostat+alvespimycin. Such combination may be of domatinostat at 100, 150, 200, 250, 300, 350, 400, 450, or 500 mg, given once or twice a day and of alvespimycin at 10, 15, 20, 25, 30, 35, or 40 mg, given once or twice a day, or every 2 days or 3 days.
Alvespimycin+daporinad. Such combination may be of alvespimycin at 10, 15, 20, 25, 30, 35, or 40 mg, given once or twice a day, or every 2 days or 3 days and of daporinad administered every 4 weeks as a continuous intravenous infusion over 96 hours at 0.018 mg/m²/h, 0.036 mg/m²/h, 0.072 mg/m²/h, 0.108 mg/m²/h, or 0.126 mg/m²/h.

In one aspect disclosed are methods for treating a patient in need using two or more drugs in combinations in dosing regimen that can target metastatic endurance of metastatic cancer cells and effectively reduce toxicity and drug exposure requirements, or cause inhibition the emergence of acquired therapeutic resistance to any single drug or cause enhanced activity or efficacy. Such drugs and dosing combinations include but are not limited to the following, when given to a patient orally as separate pills, as a fixed dose pill, or parentally, or in combination of an oral, or parenteral administration, one or more times a day, less than once a day, together or in sequence:
Rapamycin and domatinostat: Such combination may be of rapamycin at 0.5, 1, 1.5, 2, 2.5, 3, 3.5, 4, 4.5, 5, 6, 7, 8, 9 or 10 mg, given once or twice a day and of domatinostat at 100, 150, 200, 250, 300, 350, 400, 450, or 500 mg, given once or twice a day.
Rapamycin and daporinad: Such combination may be of rapamycin at 0.5, 1, 1.5, 2, 2.5, 3, 3.5, 4, 4.5, 5, 6, 7, 8, 9 or 10 mg, given once or twice a day and of daporinad daporinad given intravenously at 0.018 mg/m²/h, 0.036 mg/m²/h, 0.072 mg/m²/h, 0.108 mg/m²/h, or 0.126 mg/m²/h.
Domatinostat+daporinad. Such combination may be of domatinostat at 100, 150, 200, 250, 300, 350, 400, 450, or 500 mg, given once or twice a day and of daporinad given intravenously at 0.018 mg/m²/h, 0.036 mg/m²/h, 0.072 mg/m²/h, 0.108 mg/m²/h, or 0.126 mg/m²/h.
Rapamcyin+alvespimycin. Such combination may be of rapamycin at 0.5, 1, 1.5, 2, 2.5, 3, 3.5, 4, 4.5, 5, 6, 7, 8, 9, or 10 mg, given once or twice a day and of alvespimycin at 10, 15, 20, 25, 30, 35, or 40 mg, given once or twice a day, or every 2 days or 3 days.
Domatinostat+alvespimycin. Such combination may be of domatinostat at 100, 150, 200, 250, 300, 350, 400, 450, or 500-mg, given once or twice a day and of alvespimycin at 10, 15, 20, 25, 30, 35, or 40 mg, given once or twice a day, or every 2 days or 3 days. Alvespimycin+daporinad. Such combination may be of alvespimycin at 10, 15, 20, 25, 30, 35, or 40 mg, given once or twice a day, or every 2 days or 3 days and of daporinad administered every 4 weeks as a continuous intravenous infusion over 96 hours at 0.018 mg/m²/h, 0.036 mg/m²/h, 0.072 mg/m²/h, 0.108 mg/m²/h, or 0.126 mg/m²/h.

Due to toxicity and tolerability concerns, it may also be advantageous to optimize efficacy and safety/tolerability by using a drug combination is manner in which two drugs are used in combination for a certain time period and thereafter only one of the two drugs is administered for an extended period of time. According to one aspect, an effective and tolerable combination for treating a cancer patient in need, including by targeting minimal residual disease or preventing recurrence or targeting metastatic endurance, microscopic metastatic tumors or dormant cancer cells of using two or more drugs in combination as a fixed dose pill, or parentally, or in combination of an oral, or parenteral administration, one or more times a day, less than once a day, together or in sequence, wherein the drug combination is used in combination for a limited timeframe and thereafter only one of the two drugs is administered for an extended period of time. As examples, the combination can be given together for up to 1, 2, 3, 4, 6, 12, or 24 months and thereafter only one of the two drugs is administered for an extended period of time.

In one aspect disclosed are methods for treating such a patient in need using two or more drugs in combination, including in combinations in dosing regimen that can target metastatic endurance of metastatic cancer cells and effectively reduce toxicity and drug exposure requirements, or cause inhibition the emergence of acquired therapeutic resistance to any single drug or cause enhanced activity or efficacy, wherein domatinostat and rapamycin are given in combination, including but not limited to when given to a patient orally as separate pills, as a fixed dose pill, or parentally, or in combination of an oral, or parenteral administration, one or more times a day, less than once a day, together or in sequence, wherein the domatinostat and rapamycin drug combination is used in combination for a limited timeframe and thereafter only one of the two drugs is administered for an extended period of time. As examples, the combination can be given together for up to 1, 2, 3, 4, 6, 12, or 24 months and thereafter only one of the two drugs is administered for an extended period of time.

### 6. Assessment of Drugs for Osteosarcoma Metastasis

This study was performed to validate a 3D BME assay using paired low and high metastatic OS cell lines and to assess efficacy of drugs against metastatic endurance (ME). Two small molecular kinase inhibitors, regorafenib and saracatinib, were chosen as suspected positive and negative controls respectively given their previous clinical results in early human clinical trials. The results with these two “control drugs” provide a post hoc validation of the 3D BME assay as a predictive screen for drugs with antimetastatic activity in this adaptation of the 3D BME assay. A third small kinase inhibitor was then assessed for efficacy against ME in OS cells.

### A. Validation of Matched High and Low Metastatic OS Cell Pairs in the 3D BME Assay

Two previously described clonally related and biologically validated high and low metastatic OS cell lines MG63.3 (highly metastatic; human OS), MG63 (low metastatic, human OS), K7M2 (highly metastatic murine OS) and K12 (low metastatic murine OS), were used to optimize and validate the 3D BME assay for use with OS cells. The highly metastatic clone from each pair was then used to assess antimetastatic activity of the selected drugs.

Cells were maintained and passaged in Dulbecco's Modified Eagle Medium (DMEM)+4.5 g/L D-Glucose and 2 mM L-glutamine supplemented with 10% Fetal Bovine Serum (FBS) and 1× penicillin/streptomycin (P/S) (Gibco, Grand Island, NY), and incubated at 37° C. in 5% humidified CO₂.

Oclacitinib, saracatinib, and regorafenib (Selleck Chemical, Houston, TX) were each dissolved in DMSO at a stock concentration of 10 mM.

Previously validated high and low metastatic human OS cell lines MG63 (low metastatic) and MG63.3 (high metastatic) cells were grown at a seeding density of 400 cells/well in the same growth conditions. MG63 demonstrated a flat growth curve over the six day experiment period (consistent with a nonmetastatic phenotype). MG63.3 initially demonstrated a flat growth curve followed by an outbreak and exponential growth beginning at day 3. The growth continued in a linear fashion through the end of the study period (consistent with a metastatic phenotype). The growth from day 3-6 was significantly higher in the MG63.3 line compared to the low metastatic line.

These same validation results were found using a distinct and previously validated pair of murine OS cells K12 (low metastatic) versus K7M2 (high metastatic) cell lines. While K12 showed a flat growth curve throughout the 6-day study period, K7M2 demonstrated an initial lag phase of growth followed by outbreak and exponential growth beginning on day three. The difference in growth from day 3 forward between K12 and K7M2 was statistically significant (P<0.05).

b. Assessment of Direct Drug Cytotoxicity by Cell Number

The number of viable cells present in 2D culture was determined by MTT as previously reported. Briefly, cells were plated in 96-well plates at 2,000 cells/well and incubated at 37° C. for 24 h. After 24 h, the media was replaced with fresh media containing drugs at the desired concentrations (10 pM-10 μM) and incubated at 37° C. for 72 h. After 72 h, MTT reagent (20 L/well of a 5 mg/mL stock solution in sterile water; Sigma, St. Louis, MO) was added to each well and the plate was incubated at 37° C. for 2 h. DMSO (100 L/well) was then added to each well to solubilize the formazan crystals.

The resulting absorbance was measured at 490 nm via plate reader (Molecular Devices, San Jose, CA). DMSO (0.1%) was used as vehicle control, while 20 μM rapamycin was used as a positive cell-kill control. The experimental conditions were conducted in triplicate and each experiment was repeated three times. Regression lines and IC₅₀'s were calculated using a four-parameter variable slope nonlinear fit in GraphPad Prism.

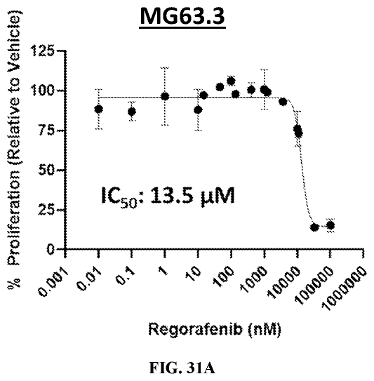

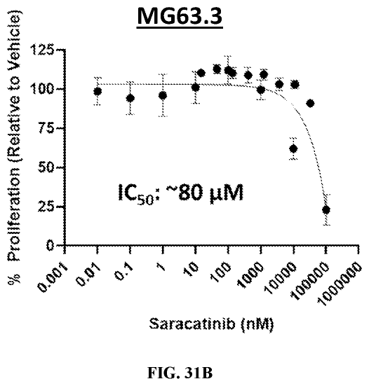

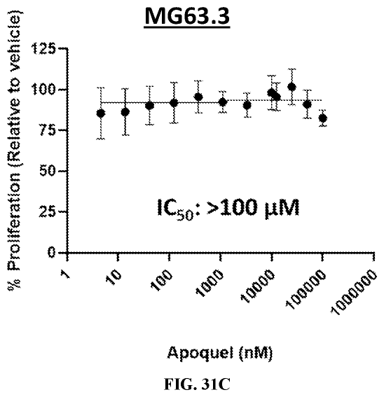

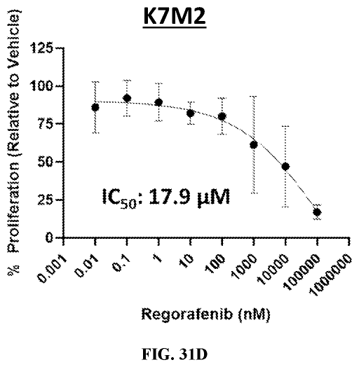

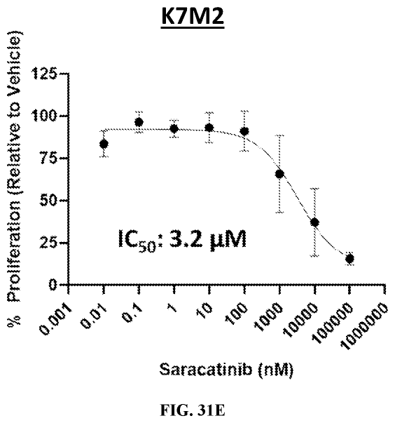

MTT assays were used to assess the influence of oclacitinib, saracatinib, and regorafenib on cell number in 2D monolayer conditions for future evaluation of exposures in the 3D BME assay that do not affect cell number but may uniquely target the metastatic phenotype. As FIG. 31 shows, collectively, exposures up to 1 μM were associated with minimal, consistent, and dose-dependent reductions in cell number across drugs and cell lines. Oclacitinib showed minimal inhibition of OS cell number in either cell line within physiologically relevant exposures up to 1 μM. Saracatinib and regorafenib showed no effect on remaining cell number over the assessed exposures in MG63.3 cells. Based on these MTT data, exposures of ≤1 μM were used in subsequent 3D BME experiments. This 1 μM exposure was also selected since such exposures are typically physiologically achievable for most small molecule inhibitors in human or preclinical studies. Accordingly, the antimetastatic activity of the drugs shown in the 3D BME assay (below 1 μM) may be described as independent of cytotoxicity.

c. Effect of Drug Treatment on OS Cells in 3D BME

The influence of drug exposure on the metastatic phenotype was assessed in an in vitro assay of ME and escape from dormancy using a 3D BME assay as previously reported, which was adapted for use with OS cells.

Cultrex® PathClear, Reduced Growth Factor Basement Membrane Extract (BME—Bio-Techne, Minneapolis, MN) was added (50 L/well) to a 96-well plate and allowed to gel by incubating at 37° C. for ≥30 min. Cells were harvested, washed with DMEM (no glucose, no FBS, Gibco, Grand Island, NY) and centrifuged at 1500 g×5 mins. The media was aspirated and the remaining cell pellet flicked for about 10 s to ensure disaggregation.

Cells were diluted in assay media containing 2% BME, resuspended vigorously and 150 μL of the cell suspension was added on top of the gelled 3D BME layer. For comparison, matched low-metastatic cell lines (MG63 & K12) were grown under the same conditions as the highly metastatic cells (MG63.3 & K7M2) and did not exhibit the same growth rate or kinetic profile, highlighting the differences in their metastatic potential. Cell numbers were determined in control vehicle (cells in media+0.1% DMSO), or treated wells and were measured on the indicated days by addition of 20 μL/well of MTS reagent (Cell Titer 96 Aqueous One, Promega, Madison, WI), followed by incubation at 37° C. for 2 h and absorbance measurements at 490 nm via plate reader (Molecular Devices, San Jose CA).

All conditions were conducted in quadruplicate and each experiment was carried out at least 3 separate times. All comparisons were made between treatment and vehicle control at concordant time points. Statistical analyses and non-linear regressions were derived using GraphPad Prism statistical software.

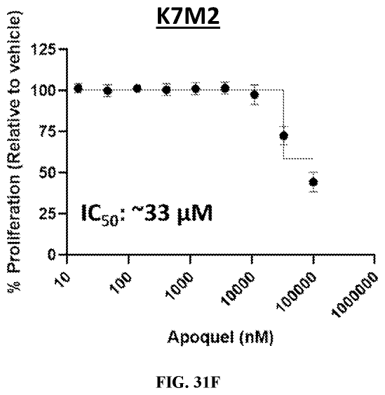

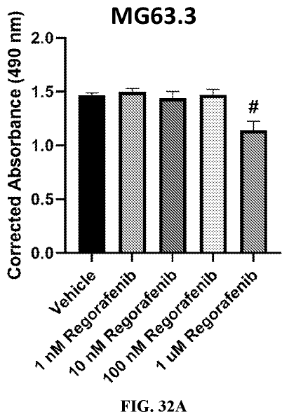

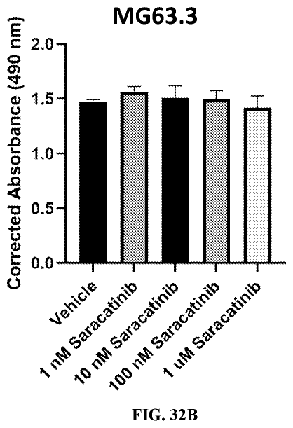

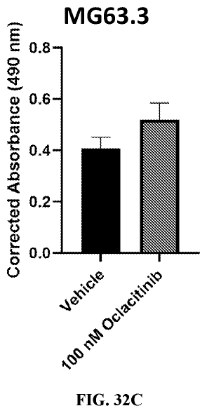

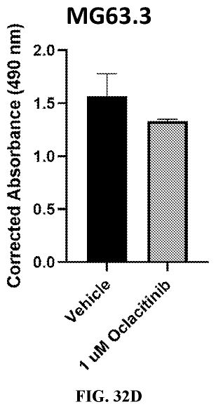

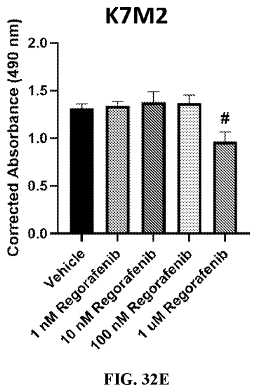

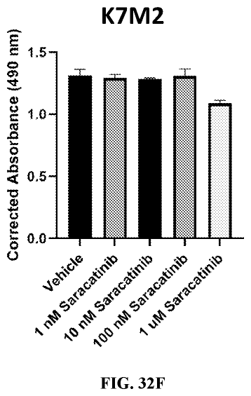

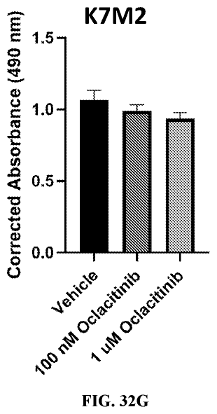

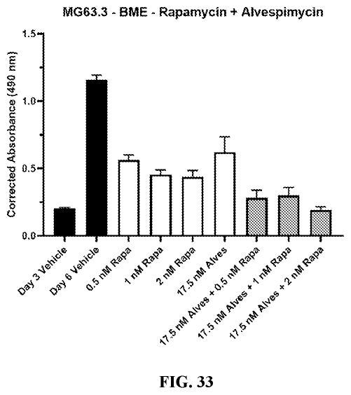

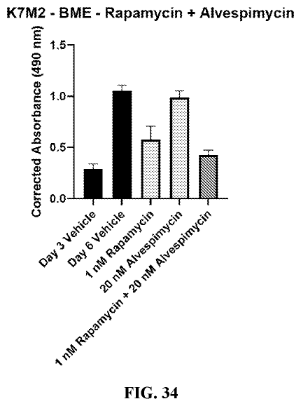

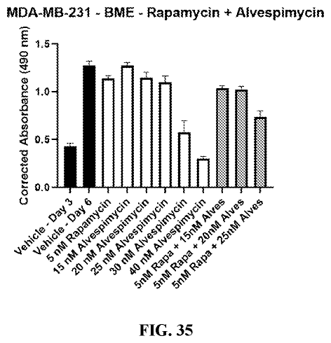

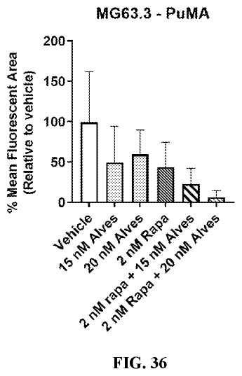

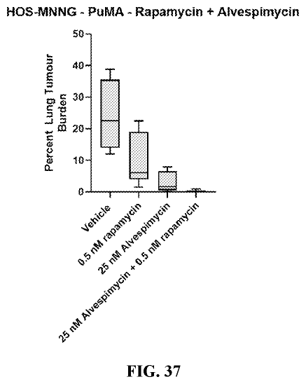

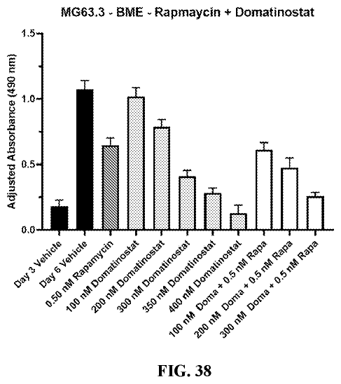

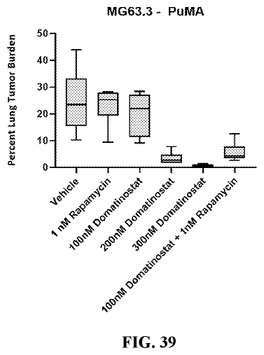

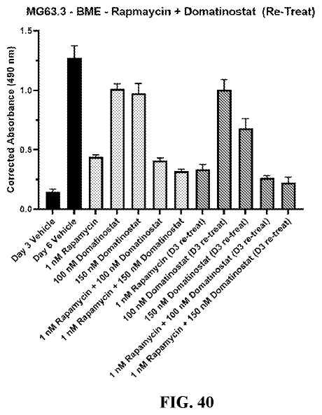

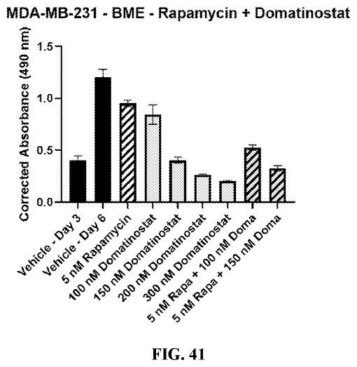

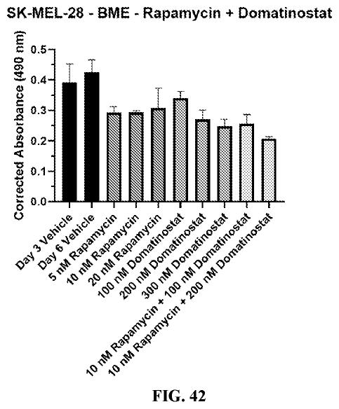

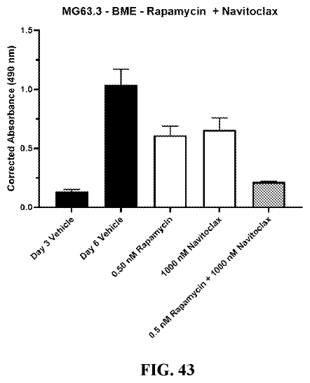

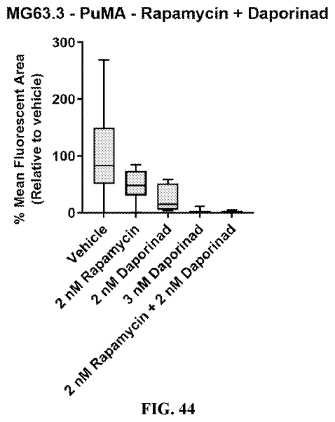

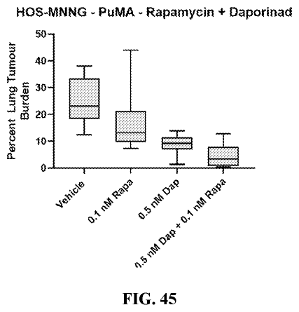

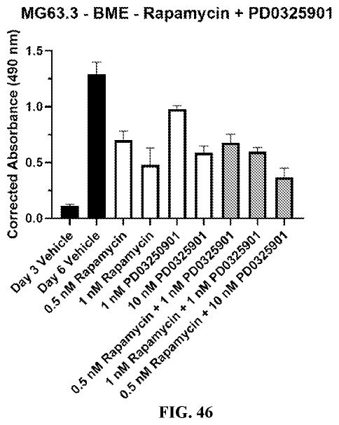

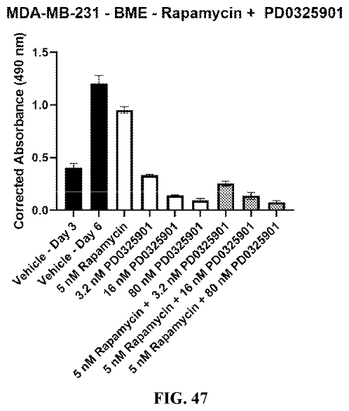

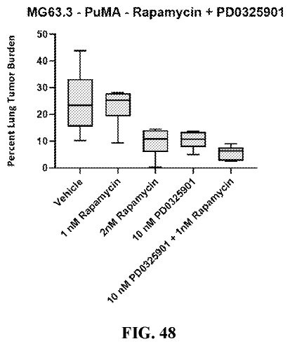

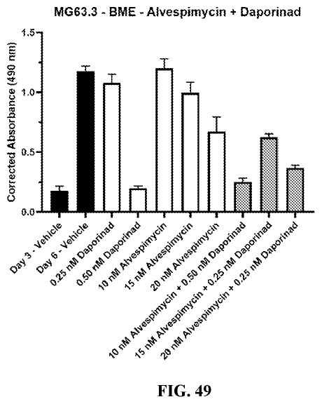

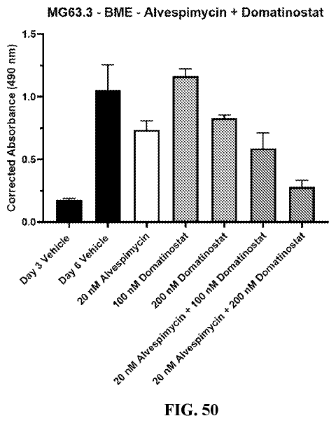

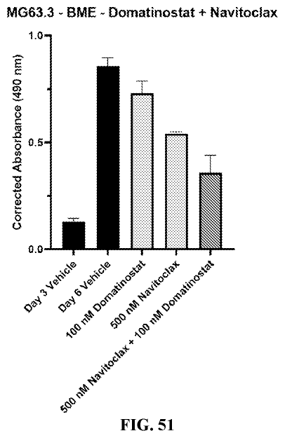

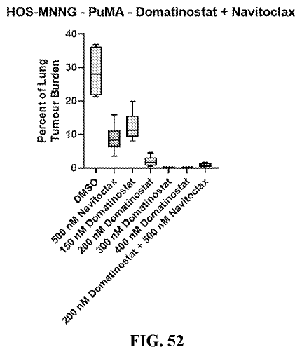

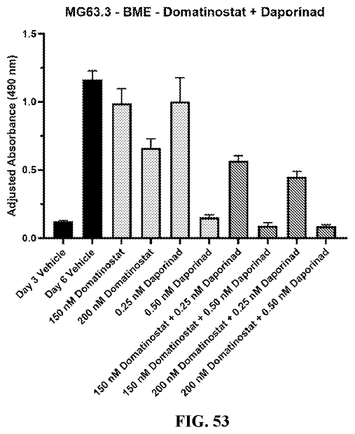

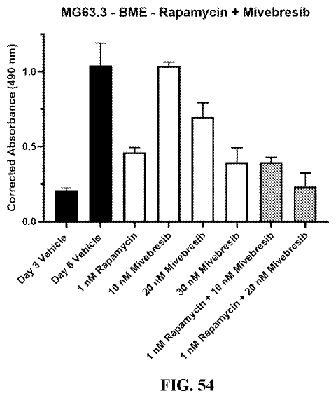

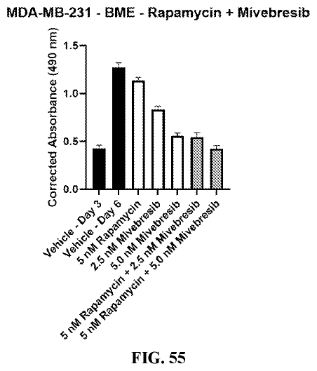

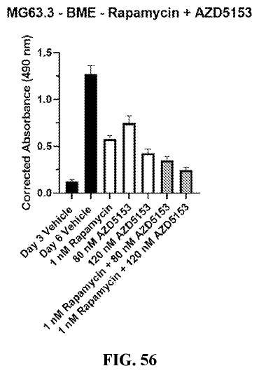

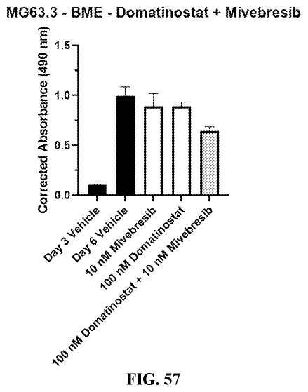

Regorafenib significantly modulated the metastatic phenotype of OS in both MG63.3 and K7M2 cell lines at physiologically relevant and non-cytotoxic exposures of 1 μM (FIG. 32; ˜20% inhibition in both cell lines, P<0.005). Significant inhibition was not seen at 100 nM. Conversely, saracatinib did not appreciably alter the metastatic phenotype of both cell lines in the 3D BME assay at or below 1 μM. Oclacitinib also had no significant effect in the 3D BME assay of metastatic phenotype in cell lines tested except for a statistically significant but very small level of inhibition at 1 μM only seen in K7M2 cells.

Regorafenib, the positive control, significantly suppressed outgrowth of cells in 3D BME at clinically relevant doses, suggestive of potential anti-metastatic activity. Regorafenib suppresses multiple receptors involved in angiogenesis (VEGFR1,2,3, TIE2 and PDGFR-B). It also has demonstrated inhibitory effects on oncogenic kinases (KIT and RET).

Saracatinib was used as a negative control for this study given its poor performance in a clinical trial in humans with progressive OS.

Oclacitinib, the drug of interest, is a Janus kinase inhibitor approved for use in controlling allergy, inflammation, and pruritus in dogs. The drug binds to JAK1/2/3 and TYK2 with its most potent inhibitory action against JAK1 (1.8 times that of JAK2 and 9.9 times that of JAK3).

Using the 3D BME assay system, screening for additive or even synergistic combinations of multiple small molecule inhibitors (or small molecule inhibitors in combination with classic cytotoxic chemotherapies) is feasible and efficient. Once identified, successful combinations of already approved FDA drugs can be rapidly translated to clinical trials in dogs or humans directly.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other methods and systems for carrying out the several purposes of this disclosure. As a result, the claims should not be considered as limited by the description or examples.

1-74. (canceled)

75. A method of preventing or inhibiting, growth or proliferation of a microscopic metastatic tumor in a subject in need thereof, the method comprising contacting the microscopic metastatic tumor with an effective amount of one or more active pharmaceutical ingredients (APIs) selected from:
(a) a mTOR inhibitor;
(b) a heat shock protein 90 (HSP90) inhibitor;
(c) a heat shock protein 70 (HSP70) inhibitor;
(d) an unfolded protein response (UPR) inhibitor;
(e) a modulator of nicotinamide adenine dinucleotide (NAD+) metabolism;
(f) a glutamyl-prolyl tRNA synthetase inhibitor;
(g) a glutaminase inhibitor;
(h) a poly(ADP)-ribose polymerase (PARP) inhibitor;
(i) a DNA-damaging agent;
(j) an agent capable of generating reactive oxygen species (ROS);
(k) a survivin inhibitors;
(l) a MEK 1 and/or MEK 2 inhibitor;
(m) a Bcl-xL and/or Bcl-2 inhibitor;
(n) a proteosome inhibitor;
(o) a muscarinic acetylcholine (mACh) antagonist;
(p) a B-Raf inhibitor;
(q) an AXL kinase inhibitor;
(r) a NEDD8 inhibitor;
(s) an aurora kinase inhibitor;
(t) a histone deacetylase (HDAC) inhibitor;
(u) a bromodomain (BET) inhibitor;
(v) a PI3K inhibitors;
(w) an Akt inhibitor;
(x) a LSD inhibitor;
(y) a DNA-PK inhibitor;
(z) an IGF1R inhibitor; and
(aa) a PLK inhibitor.

76. The method of claim 75, wherein the method comprises administering at least two APIs.

77. The method of claim 76, wherein the two APIs are administered simultaneously.

78. The method of claim 77, wherein the two APIs are co-formulated.

79. (canceled)

80. The method of claim 75, wherein the microscopic metastatic tumor is one cell or more single cells single, or one or more clusters of multiple cells that have a metastatic endurance (ME) phenotype.

81. The method of claim 75, wherein the microscopic metastatic tumor is comprised of one or more cancer cells selected from:
(a) sarcoma cells;
(b) osteosarcoma cells;
(c) osteogenic sarcoma cells;
(d) transitional cells;
(e) squamous cells;
(f) small cell carcinoma cells;
(g) medullary cells;
(h) adenocarcinoma cells; and
(i) basal cell carcinoma cells.

82. The methods of claim 75, wherein the microscopic metastatic tumor is comprised of one or more cancer cells selected from:
(a) bone cancer cells;
(b) bladder cancer cells;
(c) liver cancer cells;
(d) breast cancer cells;
(e) lung cancer cells;
(f) prostate cancer cells;
(g) pancreatic cancer cells;
(h) colon cancer cells;
(i) melanoma cells;
(j) brain cancer cells;
(k) kidney cancer cells;
(l) prostate cancer cells; and
(m) hematological cancer cells.

83. The methods of claim 75, wherein the drug is selected from rapamycin, ridaforolimus, temsirolimus, vistusertib, sapanisertib, everolimus, AZD8055, domatinostat givinostat, quisinostat, fimepinostat, panobinostat, belinostat, mivebresib, AZD5153, telaglenastat, pevonedistat, BIIB021, alvespimycin, tanespimycin, retaspimycin, talazoparib, mitomycin C, alisertib, danusertib, (E)-daporinad, minnelide, halofuginone, PD0325901, picropodophyllin, oprozomib, β-lapachone, navitoclax, bemcentinib, triptolide, dabrafenib, pimasertib, YM-155, binimetinib, AZD7648, and tropicamide.

84. A method of targeting minimal residual disease or preventing recurrence of cancer in a subject in need thereof, the method comprising administering to the subject an effective amount of an active pharmaceutical ingredient (API) selected from:
(a) a mTOR inhibitor;
(b) a heat shock protein 90 (HSP90) inhibitor;
(c) a heat shock protein 70 (HSP70) inhibitor;
(d) an unfolded protein response (UPR) inhibitor;
(e) a modulator of nicotinamide adenine dinucleotide (NAD+) metabolism;
(f) a glutamyl-prolyl tRNA synthetase inhibitor;
(g) a glutaminase inhibitor;
(h) a poly(ADP)-ribose polymerase (PARP) inhibitor;
(i) a DNA-damaging agent;
(j) an agent capable of generating reactive oxygen species (ROS);
(k) a survivin inhibitor;
(l) a MEK 1 and MEK 2 inhibitor;
(m) a Bcl-xL and Bcl-2 inhibitor;
(n) a proteosome inhibitor;
(o) a muscarinic acetylcholine (mACh) antagonist;
(p) a B-Raf inhibitor;
(q) a bromodomain (BET) protein inhibitor;
(r) an AXL kinase inhibitor;
(s) a NEDD8 inhibitor;
(t) an aurora kinase inhibitor;
(u) a histone deacetylase (HDAC) inhibitor;
(v) a PI3K inhibitor;
(w) an Akt inhibitor;
(x) a LSD inhibitor;
(y) a DNA-PK inhibitor;
(z) an IGF1R inhibitor; and
(aa) a PLK inhibitor.

85-86. (canceled)

87. The method of claim 75, wherein the API is domatinostat, givinostat, quisinostat, fimepinostat, panobinostat, belinostat, mivebresib, or AZD5153.

88-103. (canceled)

104. The method of claim 75, wherein the subject has been diagnosed as having an overt metastases.

105. The method of claim 75, wherein the subject has been diagnosed as being in partial or complete remission.

106. The method of claim 75, wherein the subject has been diagnosed as having a resectable osteosarcoma metastases.

107. The method of claim 75, wherein the subject has been diagnosed as having an unresectable osteosarcoma metastases.

108. The method of claim 75, wherein the subject has been diagnosed as having a resectable primary osteosarcoma.

109-116. (canceled)

117. The methods of claim 75, wherein the API or the drug is:
(a) rapamycin at 0.5, 1, 1.5, 2, 2.5, 3, 3.5, 4, 4.5, 5, 6, 7, 8, 9, or 10 mg, given once or twice a day;
(b) daporinad administered every 4 weeks as a continuous intravenous infusion over 96 hours at 0.018 mg/m²/h, 0.036 mg/m²/h, 0.072 mg/m²/h, 0.108 mg/m²/h, or 0.126 mg/m²/h;
(c) domatinostat at 100, 150, 200, 250, 300, 350, 400, 450, 500 or 600 mg, given once or twice a day; or
(d) alvespimycin at 10, 15, 20, 25, 30, 35, or 40 mg, given once or twice a day, or every 2 days or 3 days.

118. The method of claim 75, wherein two or more APIs or two or more drugs are administered, and wherein the two or more APIs or the two or more drugs are:
(a) rapamycin and domatinostat;
(b) rapamycin and daporinad;
(c) domatinostat and daporinad;
(d) rapamcyin and alvespimycin;
(e) domatinostat and alvespimycin; or
(f) alvespimycin and daporinad.

119. The method of claim 75, wherein two or more APIs or two or more drugs are administered, and wherein the two or more APIs or the two or more drugs are:
(a) rapamycin and domatinostat, wherein rapamycin is administered at an amount of 0.5 to 10 mg, given once or twice a day, and wherein domatinostat is administered at an amount of 100 to 600 mg per day, given once or twice a day;
(b) rapamycin and daporinad, wherein rapamycin is administered at an amount of 0.5 to 10, given once or twice a day, and wherein daporinad is administered intravenously at an amount of 0.018 mg/m²/h to 0.126 mg/m²/h for one or more days;
(c) domatinostat and daporinad, wherein domatinostat is administered at an amount of 100 to 600 mg, given once or twice a day, and wherein daporinad is administered intravenously at an amount of 0.018 mg/m²/h to 0.126 mg/m²/h;
(d) rapamcyin and alvespimycin, wherein rapamycin is administered at an amount of 0.5 to 10 mg, given once or twice a day, and wherein alvespimycin is administered at an amount of 10-40 mg, given once or twice a day or every 2 days or 3 days;
(e) domatinostat and alvespimycin, wherein domatinostat is administered at an amount of 100 to 600 mg, given once or twice a day, and wherein alvespimycin is administered at an amount of 10-40 mg, given once or twice a day or every 2 days or 3 days; or
(f) alvespimycin and daporinad, wherein alvespimycin is administered at an amount of 10-40 mg, given once or twice a day or every 2 days or 3 days, and wherein daporinad is administered intravenously at an amount of 0.018 mg/m²/h to 0.126 mg/m²/h for one or more days.

120-121. (canceled)

122. The method of claim 119, where each API is administered simultaneously.

123. The method of claim 75, wherein two or more APIs or two or more drugs are administered, and wherein the two or more APIs or the two or more drugs are:
(a) rapamycin and domatinostat, wherein rapamycin is administered at an amount of about 0.5, 1, 1.5, 2, 2.5, 3, 3.5, 4, 4.5, 5, 6, 7, 8, 9, or 10 mg, given once or twice a day, and wherein domatinostat is administered at an amount of about 100, 150, 200, 250, 300, 350, 400, 450, or 500 mg, given once or twice a day;
(b) rapamycin and daporinad, wherein rapamycin is administered at an amount of about 0.5, 1, 1.5, 2, 2.5, 3, 3.5, 4, 4.5, 5, 6, 7, 8, 9, or 10 mg, given once or twice a day, and wherein daporinad is administered intravenously at an amount of about 0.018 mg/m²/h, 0.036 mg/m²/h, 0.072 mg/m²/h, 0.108 mg/m²/h, or 0.126 mg/m²/h;
(c) domatinostat and daporinad, wherein domatinostat is administered at an amount of about 100, 150, 200, 250, 300, 350, 400, 450, or 500 mg, given once or twice a day, and wherein daporinad is administered intravenously at an amount of about 0.018 mg/m²/h, 0.036 mg/m²/h, 0.072 mg/m²/h, 0.108 mg/m²/h, or 0.126 mg/m²/h;
(d) rapamcyin and alvespimycin, wherein rapamycin is administered at an amount of about 0.5, 1, 1.5, 2, 2.5, 3, 3.5, 4, 4.5, 5, 6, 7, 8, 9, or 10 mg, given once or twice a day, and wherein alvespimycin is administered at an amount of about 10, 15, 20, 25, 30, 35, or 40 mg, given once or twice a day or every 2 days or 3 days;
(e) domatinostat and alvespimycin, wherein domatinostat is administered at an amount of about 100, 150, 200, 250, 300, 350, 400, 450, or 500 mg, given once or twice a day, and wherein alvespimycin is administered at an amount of about 10, 15, 20, 25, 30, 35, or 40 mg, given once or twice a day or every 2 days or 3 days;
(f) alvespimycin and daporinad, wherein alvespimycin is administered at an amount of about 10, 15, 20, 25, 30, 35, or 40 mg, given once or twice a day or every 2 days or 3 days, and wherein daporinad is administered every 4 weeks as a continuous intravenous infusion over 96 hours at about 0.018 mg/m²/h, 0.036 mg/m²/h, 0.072 mg/m²/h, 0.108 mg/m²/h, or 0.126 mg/m²/h; or
(g) alvespimycin and daporinad, wherein daporinad is administered every 4 weeks as a continuous intravenous infusion over 96 hours at about 0.018 mg/m²/h, 0.036 mg/m²/h, 0.072 mg/m²/h, 0.108 mg/m²/h, or 0.126 mg/m²/h.

124-133. (canceled)
